(19) United States
(12) Patent Application Publication
(10) Pub. No.: US 2026/0265382 A1
(43) Pub. Date: Sep. 10, 2026

(54) Anti-human integrin alpha3/Beta1 antibodies and methods of use therefor

(71) Applicant: MedAbome, Inc., Fremont, CA (US)

(72) Inventors: Mason Lu, Fremont, CA (US); Qinhong Ma, Fremont, CA (US); Mary Q. Xu, Houston, TX (US); Daizong Li, Clayton, MO (US); Ke-Wei Zhao, El Monte, CA (US)

(21) Appl. No.: 19/071,513

(22) Filed: Mar. 5, 2025

(51) Int. Cl. C07K 16/28; A61K 47/68; A61P 35/00

(52) U.S. Cl. CPC C07K 16/2842; A61K 47/68031; A61K 47/6849; A61K 47/6889; A61P 35/00; C07K 2317/24; C07K 2317/34; C07K 2317/73; C07K 2317/77; C07K 2317/92

(57) ABSTRACT

Disclosed is a monoclonal antibody, MAb51-31 mAb, and its fragments and derivatives that recognize a conformational epitope of human integrin α3/β1 (ITGα3/β1) complex, which is highly expressed on solid tumors but very low or absent in normal tissues. This specificity enhances its potential as a diagnostic and therapeutic agent while minimizing off-target effects. The anti-ITGα3/β1 mAb and its fragments and derivatives can be conjugated to cytotoxic drug moieties to form antibody-drug conjugates (ADCs), to inhibit tumor growth in human and animals.

### FIELD OF THE INVENTION

The present invention relates to a monoclonal antibody, MAb51-31 mAb, and its derivatives that recognize a conformational epitope of human integrin α3/β1 (ITGα3/β1) complex, which is highly expressed on solid tumors but very low or absent in normal tissues. This specificity enhances its potential as a diagnostic and therapeutic agent while minimizing off-target effects. The anti-ITGα3/β1 mAb and its derivatives can be conjugated to cytotoxic drug moieties to form antibody-drug conjugates (ADCs), can inhibit solid tumor growth in animal models.

### BACKGROUND

Integrins are a family of heterodimeric cell surface glycoproteins composed of noncovalently linked α and β subunits. At least 18α and 8β subunits are known to pair in different combinations, forming 24 distinct integrin complexes (Takada et al., 2007). Integrins mediate cell adhesion by binding to extracellular matrix (ECM) proteins such as collagen, fibronectin, and laminin. Their short cytoplasmic domains interact with actin filaments via talin, filamin, and α-actinin, serving as crucial adhesive and signal transduction molecules. Integrins play vital roles in cell adhesion, motility, proliferation, survival, and gene expression (Gullberg and Eble, 2023).

Integrin α3 (ITGA3), also known as CD49c, FRP-2, GAP-B3, GAPB3, ILNEB, JEB7, MSK18, VCA-2, VL3A, or VLA3a, exclusively pairs with the β1 subunit (ITGB1) to form the ITGα3/β1 integrin complex. In contrast, ITGB1 (or CD29) can form complexes with other 11 different integrin a subunits. The ITGα3/β1 complex, also referred to as the Epiligrin Receptor, CD49c/CD29, VLA-3, Gap b3, Galactoprotein b3, or Laminin-5 Receptor), functions as a transmembrane receptor involved in cell adhesion, migration, and intracellular signaling. ITGA3 primarily binds laminin, collagen, fibronectin, epiligrin, thrombospondin, and CSPG4, playing essential roles in epithelial tissue development, homeostasis, and repairment.

The expression of ITGα3/β1 is upregulated during wound healing, fibrosis, and in many types of cancer, including breast cancer, colorectal cancer (CRC), head and neck squamous cell carcinoma (HNSCC), gastric cancer, glioblastoma, hepatocellular carcinoma (HCC), lung cancer, melanoma, ovarian cancer, pancreatic cancer, and prostate cancer (Bijnsdorp et al, 2013; Chen et al, 2022; Jain et al, 2023; Koshizuka et al, 2017; Kuranaga et al, 2024; Linhares et al, 2015; Ramovs, 2021; Schnittert et al., 2018; Stipp, 2010; Subbaram and Dipersio, 2011; Tsuji, 2002; Zhou et al, 2014).

The role of ITGα3/β1 in cancer is multifaceted and context-depending, varying across cancer types and tumor microenvironment (TME). ITGA3 contributes to cancer progression through several mechanisms, including 1) enhancing tumor cell adhesion to the ECM, facilitating invasion and migration; 2) promoting epithelial-to-mesenchymal transition (EMT), enabling cancer cells to acquire migratory and invasive properties that drive metastasis; 3) activating multiple oncogenic pathways, including FAK-Src, PI3K/AKT, STAT3, Hippo/YAP/TAZ, and MAPK, thereby promoting cancer cell survival, proliferation, and motility (DiPersio and Longmate, 2023); 4) interacting with transforming growth factor-β (TGF-β), enhancing pro-tumorigenic and pro-fibrotic signaling (Kim et al., 2009; Margadant and Sonnenberg, 2010); and 5) supporting tumor angiogenesis and the accumulation of myofibroblasts, further contributing to tumor growth and progression (Schnittert et al., 2018). Overexpression of ITGA3 is associated with increased metastasis and poor prognosis in cancer patients, making it a potential therapeutic target and prognostic biomarker (Stipp, 2010; Subbaram and Dipersio, 2011; Schnittert, 2018; Ramovs, 2021).

Despite its critical role in cancer, no FDA-approved drugs specifically targeting ITGA3 or ITGα3/β1 are currently available. Ongoing research suggests that targeting the ITGA3 subunit or the ITGα3/β1 duplex is promising but challenging. Studies have demonstrated that silencing ITGA3 expression using tumor-suppressive microRNA-223 or small interfering RNA (siRNA) can suppress cancer cell proliferation, invasion, migration, and autophagy in esophageal squamous cell carcinoma cells (ESCC) or prostate cancer (Du et. al, 2022; Kurozumi et al, 2013). However, developing ITGA3 inhibitors remains challenging due to the structural similarities among integrin family members, the wide expression profile and physiological functions of ITGA3 in normal tissues, as well as the dual pro-tumorigenic functions of ITGα3/β1 that is, by binding to LM-332 and CD151 which complicate drug development (Ramovs et al, 2021). An alternative approach is to develop antibody drugs that specifically target aberrant ITGA3 epitopes present only in cancerous or fibrous tissues, minimizing unintended physiological disruption.

Although numerous anti-ITGA3 and ITGα3/β1 antibodies are commercially available, few have demonstrated therapeutic significance. Several groups have published research data regarding anti-ITGA3 and/or ITGα3/β1 antibodies generated with hybridomas using cancer cells as immunogens. BCMab1, a murine monoclonal antibody (mAb) targeting aberrantly glycosylated ITGα3/β1, shows potent antitumor activity in an orthotopic bladder cancer mouse model (Li et al, 2014). However, only one case report was followed (Li et al, 2017), suggesting that BCMab1 may not have met clinical expectations. KU44.22B, an anti-ITGA3 mAb has inhibitory effects on Capan-2 cells (pancreatic cancer) in vitro, but not on other cancer cells, restricting its therapeutic potential (Arias-Pinilla et al, 2020). OV-Ab 30-7 seems to be the most promising anti-ITGA3 mAb. It induces apoptosis in ovarian cancer cells by blocking ITGA3-laminin signaling, inhibiting cancer progression, and prolonging survival of tumor-bearing mice (Ke et al, 2020). However, as a murine-derived antibody, further modifications are required for clinical translation.

The high expression and aberrant post-translational modifications (PTMs) of ITGA3 on cancer cells make it a promising target for antibody-drug conjugates (ADCs). ADCs combine the specificity of mAbs to tumor associated antigens (TAAs) with the cytotoxic potency of small-molecule drugs (known as “payloads”), enabling targeted elimination of cancer cells with reduced systemic toxicity. Since the FDA approval of the first ADC, Mylotarg™, in 2000, significant advancements have been made in ADC technology, including improved cytotoxic payloads, chemical linkers, and conjugation methods. By the end of 2024, 16 ADCs have been approved globally, with over a hundred clinical trials underway. Despite these advances, identifying novel ADC targets, particularly for solid tumors and fibrotic tissues, remains a major hurdle.

A key determinant of ADC efficacy is the tumor specificity and internalization efficiency of the target antigen. In this regard, developing a high-affinity anti-ITGα3/β1 mAb that selectively binds to cancer cells while sparing human peripheral mononuclear cells (PBMCs) and normal tissues was sought.

### SUMMARY

The present disclosure provides hybridoma MAb51-31 generated by live-cell immunization (LCI) and live-cell high-throughput screening (LC-HTS) using human cancer cell lines. The MAb51-31 mAb produced by the hybridoma was shown to specifically bind to various types of cancer cell lines without cross-reacting with normal human PBMCs, bone marrow (BM) cells, or normal human tissues. MAb51-31 mAb binds to the human integrin α3/β1 complex, particularly targets a conformational epitope formed by ITGA3 and ITGB1, with a binding affinity (K_{D}) of approximately 2.5 nM.

A human-murine chimeric antibody, MAb51-31cAbm2, which contains constant regions of human kappa light chain and IgG1 heavy chain sequences, was also developed. Recombinantly expressed MAb51-31cAbm2 has similar binding properties to cancer cells and to its antigen as the murine mAb MAb51-31. When conjugated with a cleavable linker and a payload such as monomethyl auristatin E (MMAE), MAb51-31cAbm2-MMAE significantly inhibits the proliferation of multiple cancer cell lines in vitro and in vivo tumor growth in cell line-derived xenograft (CDX) mouse models. Several humanized MAb51-31 HuAb variants demonstrate similar binding affinities and recognize overlapping epitopes as does the parental MAb51-31 mAb. ADCs generated with MAb51-31HuAbm2 inhibit cancer cell proliferation in vitro.

The anti-ITGα3/β1 antibodies are preferably conjugated to MMAE through a cleavable Val-Cit-PAB linker. However, other payloads and linkers may be utilized.

The cDNA coding sequence of the variable region of the light chain of MAb51-31 mAb is shown as SEQ ID NO: 1 below.

(SEQ ID NO: 1)
AATGTTCTCACCCAGTCTCCAGCAATCTTGTCTGCATCTCCAGGGGAAAAGGTCACCATGACCT
GCAGTGCCAGCTCAAGTGTCAGTTACATGCACTGGTACCAGCAGAAGTCAGGCACCTCCCCCAA
ACTCTGGATTTATGACACATCCAAACTGCCTTCTGGAGTCCCAGGTCGCTTCAGTGGCACTGGA
TCTGGAAGTTCTTACTCTCTCACGATCAGCAGCATGGAGGCTGAAGATGTTGCCACTTATTACT
GTTTTCAGGGGAGTATGTATCCACGGACGTTCGGTGGAGGCACCACGCTGGAAATCAAACGG

The translated amino acid sequence in the variable region of the light chain of MAb51-31 mAb is shown as SEQ ID NO: 2 below. Three complementarity determining regions (CDRs1 to 3, left to right, respectively shown as SEQ ID NOs: 5, 6, 7) are highlighted in bold below and underlined.

(SEQ ID NO: 2)
ENVLTQSPAILSASPGEKVTMTCSASSSVSYMHWYQQKSGTSPKLWIYDTSKLPSGVPGRFSGT
GSGSSYSLTISSMEAEDVATYYCFQGSMYPRTFGGGTTLEIK

The cDNA coding sequence of the variable region of the heavy chain of MAb51-31 mAb is shown as SEQ ID NO: 3 below.

(SEQ ID NO: 3)
GACGTTCAGCTGCAGCAGTCTGGGGCAGAACTTGTGAAGCCAGGGGCCTCAGTCACGTTGTCCTGCAC
AGCTTCTGGCTTCAACATTAAAGACACCTATATACACTGGGTCAAACAGAGGCCTGAACAGGGCCTGG
AATGGGTTGGTAGGATTGATCCTGCGGATGGTAATACTTTATATGCCTCAAAGTTCGAGGGCCAGGCC
ACTTTAACAGCAGACTCATCATCCAACACAGCCTACATGCAACTCAGCAGCCTGACATCTGGGGACAC
TGCCGTCTATTACTGTGCTCGCCCTTATTACCATGGTGGTAGTTTCGGCTACTGGGGCCAAGGCACCA
CTCTCACAGTCTCCTCA

The translated amino acid sequence in the variable region of the heavy chain of MAb51-31 mAb or MAb51-31cAbm2 is shown as SEQ ID NO: 4 below. Three complementarity determining regions (CDRs1 to 3, left to right, respectively shown as SEQ ID NOs: 8, 9, 10) are highlighted in bold and underlined below.

(SEQ ID NO: 4)
DVQLQQSGAELVKPGASVTLSCTASGFNIKDTYIHWVKQRPEQGLEWVGRIDPADGNTLYASKF
EGQATLTADSSSNTAYMQLSSLTSGDTAVYYCARPYYHGGSFGYWGQGTTLTVSS

The translated amino acid sequence in the variable region of the light chain of MAb51-31cAbm2 is shown as SEQ ID NO: 2 above and the translated amino acid sequence in the variable region of the heavy chain of MAb51-31cAbm2 is shown as SEQ ID NO: 4 above.

The translated amino acid sequence in the variable region of the common light chain of MAb51-31HuAbm2-1, HuAbm2-3, HuAbm2-4, and HuAbm2-5 is designed as SEQ ID NO: 11 below, and three antigenic determinant regions (CDRs1 to 3, left to right, respectively shown as SEQ ID NOs: 5, 6, 7) are highlighted in bold and underlined below.

(SEQ ID NO: 11)
EIVLTQSPATLSLSPGERATLSCSASSSVSYMHWYQQKPGQAPRLWIYDTSKLPSGIPARFSGS
GSGSSYTLTISSLEPEDFAVYYCFQGSMYPRTFGGGTKVEIK

The translated amino acid sequence in the variable region of the heavy chain of MAb51-31HuAbm2-1 is shown as SEQ ID NO: 12 below, and three antigenic determinant regions (CDRs1 to 3, left to right, respectively shown as SEQ ID NOs: 8, 9, 10) are highlighted in bold and underlined.

(SEQ ID NO: 12)
EVQLVQSGAEVKKPGASVKVSCTASGFNIKDTYIHWVRQAPGQGLEWVGRIDPADGNTLYASKF
EGRVTLTADTSISTAYMELSRLRSDDTAVYYCARPYYHGGSFGYWGQGTLVTVSS

The translated amino acid sequence in the variable region of the light chain of MAb51-31HuAbm2-2 is shown as SEQ ID NO: 13 below, and three antigenic determinant regions (CDRs1 to 3, left to right, respectively shown as SEQ ID NOs: 5, 6, 7) are highlighted in bold and underlined.

(SEQ ID NO: 13)
EIVLTQSPATLSLSPGERATMTCSASSSVSYMHWYQQKPGQAPRLWIYDTSKLPSGVPARFSGS
GSGSSYTLTISSLEPEDFATYYCFQGSMYPRTFGGGTKVEIK

The translated amino acid sequence in the variable region of the heavy chain of MAb51-31HuAbm2-2 is shown as SEQ ID NO: 14 below, and three antigenic determinant regions (CDRs1 to 3, left to right, respectively shown as SEQ ID NOs: 8, 9, 10) are highlighted in bold and underlined.

(SEQ ID NO: 14)
EVQLVQSGAEVKKPGASVKVSCTASGFNIKDTYIHWVRQRPGQGLEWVGRIDPADGNTLYASKF
EGQATLTADTSISTAYMELSRLRSDDTAVYYCARPYYHGGSFGYWGQGTLVTVSS

The translated amino acid sequence in the variable region of the heavy chain of MAb51-31HuAbm2-3 is shown as SEQ ID NO: 15 below, and three antigenic determinant regions (CDRs1 to 3, left to right, respectively shown as SEQ ID NOs: 18, 19, 10) are highlighted in bold and underlined.

(SEQ ID NO: 15)
EVQLVQSGAEVKKPGASVKVSCTASGFNIKETYIHWVRQAPGQGLEWVGRIDPAEGNTLYASKF
EGRVTLTADTSISTAYMELSRLRSDDTAVYYCARPYYHGGSFGYWGQGTLVTVSS

The translated amino acid sequence in the variable region of the heavy chain of MAb51-31HuAbm2-4 is shown as SEQ ID NO: 16 below, and three antigenic determinant regions (CDRs1 to 3, left to right, respectively shown as SEQ ID NOs: 18, 9, 10) are highlighted in bold and underlined.

(SEQ ID NO: 16)
EVQLVQSGAEVKKPGASVKVSCTASGFNIKETYIHWVRQAPGQGLEWVGRIDPADGNTLYASKF
EGRVTLTADTSISTAYMELSRLRSDDTAVYYCARPYYHGGSFGYWGQGTLVTVSS

The translated amino acid sequence in the variable region of the heavy chain of MAb51-31HuAbm2-5 is shown as SEQ ID NO: 17 below, and three antigenic determinant regions (CDRs1 to 3, left to right, respectively shown as SEQ ID NOs: 8, 19, 10) are highlighted in bold and underlined.

(SEQ ID NO: 17)
EVQLVQSGAEVKKPGASVKVSCTASGFNIKDTYIHWVRQAPGQGLEWVGRIDPAEGNTLYASKF
EGRVTLTADTSISTAYMELSRLRSDDTAVYYCARPYYHGGSFGYWGQGTLVTVSS

Although the mAb MAb51-31 had some functions against cancer, when it was conjugated to a cytotoxic agent to form an ADC, it efficiently inhibited cancer growth both in vitro and in vivo. Additionally, MAb51-31 can be deployed as bispecific, multi-specific, or other fragments or antibody derivatives, or dual-drug ADCs, antibody radionuclide conjugates (RACs), CAR-T/NK/microphage/DC therapies, and proteolysis-targeting chimera (PROTAC), offering innovative solutions for other therapeutic applications in the treatment of fibrosis and neural diseases.

The MAb51-31 ADCs can also be used alone or in combination with other therapeutic approaches for the treatment of cancers, such as breast, lung, colorectal, gastric, pancreatic, or skin cancer, particularly those resistant to standard anti-cancer therapies, including triple-negative breast cancer, late-stage pancreatic, lung, gastrointestinal or colorectal cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1 to 1A-6 show that MAb51-31 mAb binds to 33 of 35 tested human solid tumor cell lines on cell surfaces in FACS analyses. The meaning of the abbreviations in the figures is: MFI: median fluorescence intensity; FITC: fluorescein isothiocyanate; [mAb]: concentration of monoclonal antibody; NSCLC: non-small cell lung cancer; SCLC: small cell lung cancer; GC: gastric cancer; PDAC: pancreatic ductal adenocarcinoma; CC: cervical cancer; HCC: hepatocellular carcinoma; CRC: colorectal cancer; TNBC: triple-negative breast cancer; BC: breast cancer.

FIGS. 1B-1 and 1B-2 depict that MAb51-31 mAb binds to two of 12 tested hematologic malignant cell lines. The meaning of the abbreviations in the figures is: AML: acute myeloid leukemia; CML: chronic myeloid leukemia; T-ALL: T cell acute lymphoblastic leukemia; B-ALL: B cell acute lymphoblastic leukemia; NHL-BL: non-Hodgkin lymphoma (Burkitt lymphoma).

FIG. 2A shows no interaction between MAb51-31 mAb and normal human peripheral blood mononuclear cells (PBMCs), including a general population and subpopulations of lymphocytes, monocytes, neutrophils, and platelets.

FIG. 2B illustrates that MAb51-31 mAb does not bind to normal human bone marrow mononuclear cells (BMMCs), or any of the gated populations based on size and internal complexity. BMMCs were categorized using a forward scatter (FSC) vs. side scatter (SSC) plot to distinguish large cells, medium cells with high complexity (high-medium), medium cells with low complexity (low-medium), and small cells.

FIG. 2C shows no interaction between MAb51-31 mAb and normal human cord blood mononuclear cells (CBMCs), including a general population and subpopulations of lymphocytes, monocytes, neutrophils, and platelets.

FIG. 2D demonstrates no interaction between MAb51-31 mAb and normal cynomolgus monkey PBMCs, including a general population and subpopulations of lymphocytes, monocytes, neutrophils, and platelets.

FIG. 3A shows the IHC result of formalin-fixed, paraffin-embedded (FFPE) NSCLC section reacted with MAb51-31 mAb. Tissue was visualized under a microscope at 10× magnification. Blue staining corresponds to hematoxylin counterstaining, which visualizes cells and nuclei, while positive staining appears in brown.

FIG. 3B displays the IHC results of normal human tissue microarray (TMA) stained with MAb51-31 mAb (upper panel) and a table summarizing the staining results along with the corresponding tissue locations on the TMA slide (lower panel). The TMA slide contains 90 different sections, with very three consecutive spots being the same tissue from three distinct donors. Placenta serves as a positive control. Blue staining corresponds to hematoxylin counterstaining, which visualizes cells and nuclei, while positive staining appears in brown.

FIGS. 4A and 4B show the scores and sequence coverages of ITGB1 (FIG. 4A) and ITGA3 (FIG. 4B) identified through three independent immunoprecipitation (IP) and liquid chromatography with tandem mass spectrometry (LC-MS/MS) analyses. Accession numbers correspond to UniProtKB/Swiss-Prot IDs, and deduced peptide sequences are highlighted in bold. PEP: posterior error probability; PSMs: peptide spectrum matches; #AAs: number of amino acids. The peptide sequences derived from the LC-MS/MS results are shown in bold.

FIGS. 5A to 5D illustrate a strong interaction between MAb51-31 mAb and recombinant human integrin α3/β1 complexes, a weak interaction with the rhITGA3 monomer, and no interaction with the rhITGB1 monomer in ELISA assays.

FIG. 5A shows the dose-dependent binding of MAb51-31 mAb with 0.15 μg/mL of immobilized rhITGα3/β1 complex expressed in CHO cells (R&D Systems). Neither the anti-6×His mAb nor the isotype control mAb bound to the antigens. A₄₅₀ represents absorbance at 450 nm. Error bars indicate standard deviation (SD) of duplicate samples.

FIG. 5B illustrates the dose-dependent binding of MAb51-31 mAb to 0.15 μg/mL of immobilized rhITGα3/β1-His complex expressed in HEK293 cells (Acro Biosystems), with anti-6×His mAb serving as a positive control. A₄₅₀ represents absorbance at 450 nm. Error bars indicate standard deviation (SD) of duplicate samples.

FIG. 5C depicts the weak interaction between MAb51-31 mAb and rhITGA3-FLAG expressed HEK293 cells (Origene Technologies).

FIG. 5D shows no detectable interaction between MAb51-31 mAb and rhITGB1-ECD-His expressed HEK293 cells (SinoBiological), with anti-6×His mAb serving as a positive control.

FIG. 6 shows no detectable interaction between MAb51-31 mAb and rhITGB2, rhITGαL/β2, rhITGαV/β3, or rhITGαV/β6 in ELISA assays.

FIG. 7 presents fitted aligned sensorgram traces depicting the association and dissociation curves for MAb51-31 mAb binding to rhITGα3/β1-His, extracted from raw data analyzed by the ForteBio Octet® QK^{e} System. Anti-Mouse Fc Capture (AMC) biosensors were immobilized with 50 nM MAb51-31 mAb. Recombinant human ITGα3/β1-His was loaded at concentrations of 100, 50, 25, and 12.5 nM by 2-fold serial dilution to allow association and dissociation for 15 min and 30 min, respectively.

FIG. 8 shows the relative expression levels of the ITGA3 gene in representative cancer cell lines, as determined by qRT-PCR analysis. Data were normalized to RPLP0 mRNA and expressed as fold change relative to healthy human PBMC controls. Values represent the mean±SD of triplicate samples. *P<0.05, **P<0.01 (Student's t test).

FIG. 9 illustrates the effect of MAb51-31 mAb on the invasion of various cancer cell lines. ctrl represents the baseline of isotype control mAb-treated groups. Positive values indicate increased invasion, while negative values indicate decreased invasion. Data are represented as the mean±SD of triplicate samples.

FIGS. 10A to 10G are fluorescent images showing internalization of FITC-labeled MAb51-31 mAb in seven different human cancer cell lines. Cells were incubated with 1 μg/mL MAb51-31 mAb-FITC, or an isotype control mAb-FITC for one hour at 4° C. After PBS wash, the cells were incubated at 37° C. with 5% CO₂ for 15 min, 30 min, or 2 hours. Location and intensity of green fluorescence were observed under Keyence Fluorescence Microscope. The dynamics of antibody binding and internalization of each antibody are shown vertically, with the 4° C. condition as the baseline of antibody binding. The pictures were taken at 40× magnification with the same exposure time. Scale bars indicate 100 μm.

FIG. 11 illustrates the dose-dependent binding of MAb51-31cAbm2 to 0.15 μg/mL of immobilized rhITGα3/β1-His complex in an ELISA assay. cAbm2: chimeric antibody with double cysteine mutations in each heavy chain constant region (Fc); [Ab]: antibody concentration.

FIG. 12 illustrates the dose-dependent binding of MAb51-31cAbm2 to the surface of various human cancer cell lines determined by FACS analysis.

FIG. 13 is the fitted sensorgram traces showing association and dissociation curves for MAb51-31cAbm2 binding to rhITGα3/β1-His, extracted from raw data by the ForteBio software. The Octet® QK^{e} System was equipped with Anti-Human Fc Capture (AHC) biosensors to immobilize 50 nM MAb51-31cAbm2. Recombinant human integrin «3/β1 was loaded at concentrations of 100, 50, 25, and 12.5 nM by 2-fold serial dilution to allow association and dissociation for 15 min and 30 min, respectively.

FIG. 14 shows the SDS-PAGE analysis of MAb51-31cAbm2 with (+) or without (−) MMAE conjugation under both non-reducing and reducing conditions. Numbers to the left indicate the molecular weights (kDa) of Broad Multi Color Pre-Stained Protein Standard (GenScript).

FIG. 15 illustrates the surface binding of MMAE-conjugated and unconjugated MAb51-31cAbm2 to various cancer cell lines in FACS analyses. An isotype control chimeric antibody (Isotype ctrl cAbm2), with or without MMAE conjugation, served as the negative control. [Ab]: antibody concentration.

FIGS. 16-1 and 16-2 illustrate the inhibitory effects of MAb51-31cAbm2-MMAE on the proliferation of various human cell lines, as determined by cell counting assays. The non-cell binding isotype control, Isotype ctrl cAbm2-MMAE, served as the proliferation baseline for each cell line. HepG2 and HEK293 cells, which do not express integrin α3β1, were used as negative controls. A₄₅₀: absorbance at 450 nm. Data represent the mean±SD of triplicate samples.

FIGS. 17A to 17F illustrate the antitumor effects of MAb51-31cAbm2-MMAE on six cancer cell line-derived xenograft (CDX) mouse models. MAb51-31cAbm2-MMAE, isotype control cAbm2-MMAE, or PBS was injected intraperitoneally (i.p.) with the dose as indicated on Day 0. Data represents average tumor volume±SD of five mice per group (n=5). PBS: Dulbecco's phosphate buffer saline; MMAE: monomethyl auristatin E.

FIG. 18A depicts the comparison of the parental murine mAb light chain variable region (MoVL) with the select human germline acceptor IGKV3-11*01 and two humanized MAb51-31 light chain VLs (HuVL1 and HuVL2). Grey and black bars on top of the sequences are the Kabat and Chothia definitions of CDRs. Amino acids in CDRs are highlighted in bold and underline. Back mutations in frameworks are highlighted in grey and italicized.

FIG. 18B depicts the comparison of the parental murine mAb heavy chain variable region (MoVH) with the select human germline acceptor IGHV1-2*02 and five humanized MAb51-31 heavy chains (HuVH1 to HuVH5). Grey and black bars on top of the sequences are the Kabat and Chothia definitions of CDRs. Amino acids in CDRs are highlighted in bold and underline. Back mutations in frameworks are italicized. The D31E mutation in CDR-H1 and D55E mutation in CDR-H2 are also highlighted in grey.

FIG. 18C shows different combinations of HuVL and HuVH pairs for expressing five different humanized MAb51-31HuAbm2s. All back mutations and the D-to-E point mutations in CDR-H1 and CDR-H2 are listed and their differences are marked in bold.

FIG. 19 shows the curves of dose-dependent binding to six representative cancer cell lines by MAb51-31HuAbm2-1 and -HuAbm2-2 are comparable with that of MAb51-31cAbm2, as determined by FACS analysis.

FIG. 20 shows a competition ELISA assay, where binding between rhITGα3/β1-His and HRP-conjugated MAb51-31 mAb (MAb51-31mAb-HRP) was inhibited by unlabeled MAb51-31cAbm2, HuAbm2, to a similar extent as their parental murine MAb51-31 mAb. The isotype control cAbm2, which recognizes a non-cell surface antigen, did not compete with MAb51-31mAb-HRP. The plate was immobilized with 0.2 μg/mL rhITGα3/β1-His.

FIG. 21 shows the fitting view of the association and dissociation curves and the data table of the binding kinetics between MAb51-31HuAbm2 and rhITGα3/β1-His at 2-fold serial dilutions (100, 50, 25, and 12.5 nM).

FIGS. 22A to 22F are fluorescent images showing internalization of CF488-conjugated MAb51-31HuAbm2 by six different human cancer cell lines. Cells were incubated with 1 μg/mL MAb51-31HuAbm2-CF488, or an isotype control cAbm2-CF488 for one hour at 4° C. to obtain the baselines of antibody binding for each cell line. Antibody internalization was observed at 15 min, 30 min, and 2 hours after incubation at 37° C. The pictures were taken at 40× magnification with the same exposure time. Scale bars indicate 100 μm.

FIG. 23 shows the SDS-PAGE analysis of MAb51-31HuAbm2 with (+) or without (−) MMAE conjugation under both non-reducing and reducing conditions. Numbers to the left indicate the sizes of the bands in molecular weight marker.

FIG. 24 compares the surface binding of MAb51-31cAbm2 and MAb51-31HuAbm2, with or without MMAE conjugation, to six human cancer cell lines, as determined by FACS analyses. Each cell line represents a distinct cancer type.

FIG. 25 shows in vitro cell counting assays comparing the efficacies of MMAE-conjugated MAb51-31cAbm2 and HuAbm2 in inhibiting the proliferation of four representative human cancer cell lines. Isotype control cAbm2-MMAE served as the proliferation baseline for each cell line. Data represent the mean±SD of triplicate samples.

### DETAILED DESCRIPTION

### Definitions

The section headings used herein are for organizational purposes only and are not to be construed as limiting the subject matter described. All patents and applications listed herein are incorporated by reference.

Unless defined otherwise, all technical and scientific terms used herein have the same meaning as is commonly understood by one of ordinary skill in the art. All patents, applications, published applications, and other publications are incorporated by reference in their entirety. In the event that there is a plurality of definitions for a term herein, those in this section prevail unless stated otherwise.

The term “ADCs” refers to antibody-drug conjugate, wherein a monoclonal antibody is conjugated with a drug. Suitable drugs in an ADC include any substance having biological or detectable activity, for example, therapeutic agents, detectable labels, binding agents, and prodrugs, which are metabolized to an active agent in vivo. Suitable drugs in the conjugates preferably include anti-tumor agents, including Monomethyl auristatin E (MMAE) or another anti-tumor agent, for example, a cytotoxin, including maytansine or a derivative thereof, or a derivative of Auristatin, epothilone or a derivative thereof, paclitaxel or a derivative thereof, or a vinca alkaloid compound; or further including: Combretastatin A-4 phosphate, Combretastatin A-4 and its derivatives, indole-sulfa compounds, vinca alkaloids compounds such as vinblastine, vincristine, vindesine, vinorelbine, vinflunine, vinglycinate, anhydrovinblastine, dolastatins 10 and analogues, halichondrin B and Eribulin, indole-3-oxalyl amides, substituted indol-3-oxalyl amides, podophyllotoxins, 7-diethylamino-3-(2′-benzoxazolyl)-coumarin (DBC), discodermolide, Laulimalide; DNA topoisomerase inhibitors such as camptothecin and its derivatives, mitoxantron; mitoguazone; nitrogen mustard analogues such as Chlorambucil, Chlomaphazine, cyclophosphamide, Estramustine, ifosfamide, Mustine, Nitromin, Melphalan, Novembichin, Phenamet, Phenesterine, Prednimustine, Trofosfamide, Uramustine; nitrosoureas such as Carmustine, streptozotocin, Fotemustine, Lomustine, Nimustine, Ranimustine; antibiotics such as the enediyne antibiotics, Dynemicin, Esperamicin, Neocarzinostatin, Aclacinomycin, Actinomycin, Anthroamycin, Azaserine, Bleomycins, actinomycin C, Carabicin, Idarubicin, Carzinophilin, Carminomycin, Actinomycin D, Daunorubicin, Doxorubicin, 6-diazo-5-oxo-L-norleucine, Adriamycin, Epirubicin, Esorubicin, Idarubicin, Marcellomycin, Mitomycins, Mycophenolic acid, Nogalamycin, Olivomycin, Peplomycin, Bofeimeisu, Puromycin, Adriamycin-Fe, Rodorubicin, Streptonigrin, Streptozocin, Tubercidin, Ubenimex, Zinostatin, Zorubicin; folic acid analogues such as Denopterin, Methotrexate, Pteropterin, Trimetrexate, Edatrexate; Purine analogues such as Fludarabine, 6-mercaptopurine, Thiamiprine, Thioguanine; pyrimidine analogues such as Ancitabine, Gemcitabine, Enoxaparin, Azacitidine, 6-Azauridine, Carmofur, Cytarabine, dideoxyuridine, deoxy-fluorouridine, Fluoruridine; androgens such as Calusterone, Dromostanolone propionate, Epitiostanol, Mepitiostance, Testolactone; anti-adrenal compounds such as Aminoglutethimide, Mitotane, Trilostane; trichothecenes such as T-2 toxin, verracurin A, Roridin A and Anguidine; arizidines such as Benzodopa, Carboquone, Meturedopa and Uredopa; platinum analogs such as Cisplatin, Carboplatin, Oxaliplatin, Miriplatin, Etoposide; anti-androgens such as Flutamide, Nilutamide, Bicalutamide, Leuprolide and Goserelin; protein kinase and proteasome inhibitors.

The antibody is preferably bound to the anti-tumor agent using a linker, or other binding methods can be used. In some embodiments, linkers may be used in the conjugate bonding which can be composed of flexible residues like glycine and serine (one example being four glycine residues followed by a serine, repeated; or simply repeated glycine residues) so that the adjacent protein domains are free to move relative to one another. If one desires to maintain distance between domains, so they cannot interact, rigid linkers are preferred; one example being (repeating): glutamic acid, three alanine residues, lysine. Another rigid linker is (repeating): any amino acid residue and proline. Linkers can be either non-cleavable (e.g., thioether, SMCC, PEG linkers), or cleavable linkers, such as valine-citrulline (Val-Cit, VC) dipeptide, glutamic acid-valine-citrulline (Glu-Val-Cit, GVC) tripeptide and disulfide linkers. U.S. Pat. No. 9,310,373 “Molecular conjugate” discloses a number of hydrazide thiol linkers, and making conjugates with them. U.S. Pat. No. 8,518,891 discloses a linker with an aminoarylmethyl or aminoheteroaryl moiety. Bi-functional linkers have also been described. See U.S. Pat. No. 11,040,084.

The antibody in the ADC can be a whole antibody or a fragment thereof. The Binding Agents of the invention include MAb51-31-like or MAb51-31-derived antibodies, antibody fragments, fusion proteins, or an antibody-derived or modified chimeric antibody receptor (CAR), including single-chain variable fragments (scFv) fused to intracellular signaling domains, e.g., the zeta chain of CD3 (CD32). CAR is preferably expressed in CAR immune effector cells including but not limited to T cells, natural killer (NK) cells, microphages, and dendritic cells (DCs).

The term Binding Agent(s) includes an antibody (both are interchangeably used in singular or plural form) which is an immunoglobulin molecule capable of binding to a target antigen, such as an antigen on lung cancer cells, through at least one antigen recognition site, located in the variable region of the immunoglobulin molecule. As used herein, the term “antibody” encompasses not only intact (i.e., full-length) monoclonal antibodies like MAb51-31, but also antigen-binding fragments thereof (such as Fab, Fab′, F(ab′)₂, Fv, Fd, rIgG, single chain (scFv) or sc(Fv)₂, mutants thereof, fusion proteins comprising an antibody portion, humanized antibodies (like MAb51-31HuAbm2), chimeric antibodies (like MAb51-31cAbm2), diabodies, multispecific antibodies (e.g., bispecific antibodies), single domain antigen binding (SDAB) molecules, a VH or VL domain, or a VHH domain, and any other modified configuration of the immunoglobulin molecule that comprises an antigen recognition site of the required specificity, including glycosylation variants of antibodies, amino acid sequence variants of antibodies, and covalently modified antibodies. Also included are antibody-drug conjugates.

An antibody includes antibodies of any class, such as IgD, IgE, IgG, IgA, or IgM (or a sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1 and IgA2.

Antibodies described herein can be murine, rat, human, or any other origin (including chimeric or humanized antibodies, including as described in U.S. Pat. No. 7,317,091B2, and including such antibodies generated by affinity maturation). In some examples, the antibody comprises a modified constant region, such as a constant region that is immunologically inert, e.g., does not trigger complement mediated lysis, or does not stimulate antibody-dependent cell mediated cytotoxicity (ADCC).

Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody will also optimally comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody. Humanized antibodies may also involve affinity maturation. See Jones et al., Nature, 321:522-525 (1986); Riechmann et al., Nature, 332:323-327 (1988); Verhoeyen et al., Science, 239:1534-1536 (1988).

A chimeric antibody includes a heavy chain constant region and a light chain constant region from a human antibody. See, e.g., Morrison et al. (1984) Proc. Natl. Acad. Sci. USA 81, 6851; Neuberger et al. (1984) Nature 312, 604; and Takeda et al. (1984) Nature 314:452. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. See U.S. Pat. No. 4,816,567.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

### Formulations

After antibodies and Binding Agents and ADCs can be prepared in a formulation for administration to a subject. A lyophilized formulation is preferred, which as a first step, requires preparing a pre-lyophilized formulation. The amount of ADC in the pre-lyophilized formulation is determined taking into account the desired dose volumes, mode(s) of administration, etc. The protein is generally present in solution. For example, the protein may be present in a pH-buffered solution at a pH from about 4-8, and preferably from about 5-7. Exemplary buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the formulation (e.g. of the reconstituted formulation). The preferred buffer is histidine as it can have lyoprotective properties. Succinate is also a useful buffer.

The lyoprotectant is added to the pre-lyophilized formulation. In preferred embodiments, the lyoprotectant is a non-reducing sugar such as sucrose or trehalose. The amount of lyoprotectant in the pre-lyophilized formulation is generally such that, upon reconstitution, the resulting formulation will be isotonic, as preferred, though hypertonic reconstituted formulations may also be suitable. In addition, the amount of lyoprotectant must not be too low such that an unacceptable amount of degradation/aggregation of the protein occurs upon lyophilization.

Where the lyoprotectant is a sugar (such as sucrose or trehalose) and the protein is an antibody, exemplary lyoprotectant concentrations in the pre-lyophilized formulation are from about 10 mM to about 400 mM, and preferably from about 30 mM to about 300 mM, and most preferably from about 50 mM to about 100 mM.

The ratio of protein to lyoprotectant is selected for each protein and lyoprotectant combination. In the case of an antibody as the protein of choice and a sugar (e.g., sucrose or trehalose) as the lyoprotectant for generating an isotonic reconstituted formulation with a high protein concentration, the molar ratio of lyoprotectant to antibody may be from about 100 to about 1500 moles lyoprotectant to 1 mole antibody, and preferably from about 200 to about 1000 moles of lyoprotectant to 1 mole antibody, including from about 200 to about 600 moles of lyoprotectant to 1 mole antibody.

In preferred embodiments, it has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68 etc). The amount of surfactant added is such that it reduces aggregation of the reconstituted protein and minimizes the formation of particulates after reconstitution. For example, the surfactant may be present in the pre-lyophilized formulation in an amount from about 0.001-0.5%, and preferably from about 0.005-0.05%.

A mixture of the lyoprotectant (such as sucrose or trehalose) and a bulking agent (e.g. mannitol or glycine) may be used in the preparation of the pre-lyophilization formulation. The bulking agent may allow for the production of a uniform lyophilized cake without excessive pockets therein.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include; additional buffering agents; preservatives; co-solvents; antioxidants including ascorbic acid and methionine; chelating agents such as EDTA; metal complexes (e.g. Zn-protein complexes); biodegradable polymers such as polyesters; and/or salt-forming counterions such as sodium. In addition, various adjuvants such as are commonly used in the art may be included. Considerations for the inclusion of various components in pharmaceutical compositions are described, e.g., in Gilman et al. (Eds.) (1990), Goodman and Gilman's: The Pharmacological Basis of Therapeutics, 8th Ed., Pergamon Press, which is incorporated herein by reference in its entirety.

The pharmaceutical compositions and formulations described herein are preferably stable, so as to retain its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for protein, at about 120° C. for about 30 minutes.

After the protein, lyoprotectant and other optional components are mixed together, the formulation is lyophilized. Many different freeze-dryers are available for this purpose such as Hull50® (Hull, USA) or GT20® (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation.

Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hours). A secondary drying stage may be carried out at about 0-40° C., depending primarily on the type and size of container and the type of protein employed. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial. As a general proposition, lyophilization will result in a lyophilized formulation in which the moisture content thereof is less than about 5%, and preferably less than about 3%.

At the desired stage, typically when it is time to administer the protein to the patient, the lyophilized formulation may be reconstituted with a diluent such that the protein concentration in the reconstituted formulation is preferably similar to that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%.

Alternatively, a non-lyophilized formulation may be used, including ADCs and any of the well-known carriers, excipients, buffers, stabilizers, preservatives, adjuvants and other additives described herein and well known in the art.

### Dosages and Administration

The ADC formulations described above can be administered to a subject (e.g., a human) in need of the treatment via a suitable route, such as administration by intravenous, intramuscular, intraperitoneal, intracerebrospinal, subcutaneous, intracutaneous, intraarticular, intrasynovial, intrathecal, intradermal, intratumoral, intranodal, intramedulla, oral, inhalation or topical routes; or it may be administered orally, by inhalation spray, topically, rectally, nasally, buccally, vaginally or via an implanted reservoir; and in any case, as a bolus or by continuous infusion over a period of time; or via injectable depot routes of administration such as using 1-, 3-, or 6-month depot injectable or biodegradable materials and methods.

Commercially available nebulizers for liquid formulations, including jet nebulizers and ultrasonic nebulizers are useful for administration. Liquid formulations can be directly nebulized and lyophilized powder can be nebulized after reconstitution. Alternatively, ADCs can be aerosolized using a fluorocarbon formulation and a metered dose inhaler, or inhaled as a lyophilized and milled powder. Where CAR is deployed in the invention, compositions of immune effector cells (e.g., T cells, NK cells) may be injected directly into a tumor, lymph node, or site of infection, or elsewhere.

The subject to be treated by the methods described herein can be a mammal, more preferably a human. Mammals include, but are not limited to, farm animals, sport animals, pets, primates, horses, dogs, cats, mice and rats.

An “effective amount” refers to the amount of ADC required to confer therapeutic effect on the subject, either alone or in combination with one or more other active agents. Effective amounts vary, depending on the particular condition being treated, the severity of the condition, the individual patient parameters including age, physical condition, size, gender and weight, the duration of the treatment, the nature of concurrent therapy (if any), the specific route of administration and like factors, all of which are well known to those of ordinary skill in the art and can be addressed with no more than routine experimentation. It is generally preferred that a maximum dose of the individual components or combinations thereof be used, that is, the highest safe dose according to sound medical judgment. A lower dose or tolerable dose for medical reasons, psychological reasons or other reasons, is also appropriate.

Empirical considerations, such as the ADC half-life, generally will contribute to the determination of the dosage. For example, ADCs that are compatible with the human immune system, e.g., those including humanized antibodies or fully human antibodies, may be used to prolong half-life of the antibody and to prevent the ADC being attacked by the host's immune system. Frequency of administration may be determined and adjusted over the course of therapy, and is generally, but not necessarily, based on treatment and/or suppression and/or amelioration and/or delay of cancer. Alternatively, sustained continuous release formulations of antibody may be appropriate. Various formulations and devices for achieving sustained release are known in the art.

In one example, dosages for an ADC as described herein may be determined empirically in individuals who have been given one or more administration(s) of the antibody. Individuals are given incremental dosages of the antibody. To assess efficacy of the ADC, an indicator of the disease (e.g., tumor growth) can be followed according to routine practice.

Generally, for administration of any of the ADCs described herein, an initial candidate dosage can be extrapolated from the experiments described below. For repeated administrations over several days or longer, depending on the condition, the treatment is sustained until a desired suppression of symptoms occurs or until sufficient therapeutic levels are achieved to alleviate the cancer. An exemplary dosing regimen comprises administering an initial higher dose, followed by a lower maintenance dose. However, other dosage regimens may be useful, depending on the pattern of pharmacokinetic decay that the practitioner wishes to achieve. For example, dosing from one-four times a week is contemplated. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

Conventional methods, known to those of ordinary skill in the art of medicine, can be used to administer the pharmaceutical composition to the subject, depending upon the treatment goal and the cancer site.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like).

For intravenous injection, water soluble ADCs can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients.

Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an ADC is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., WO 00/53211 and U.S. Pat. No. 5,981,568,

In another embodiment of the present disclosure, an article of manufacture is provided which contains any of the pharmaceutical compositions and formulations described herein (e.g., comprising an ADC) and provides instructions for its use and/or reconstitution. The article of manufacture comprises a container. Suitable containers include, for example, bottles, vials (e.g., dual chamber vials), syringes (such as dual chamber syringes) and test tubes. The container may be formed from a variety of materials such as glass or plastic. The container holds the formulation and the label on, or associated with, the container may indicate directions for reconstitution and/or use. For example, the label may indicate that the formulation is reconstituted to particular protein concentrations. The container holding the formulation may be a multi-use vial, which allows for repeat administrations (e.g., from 2-6 administrations) of the reconstituted formulation. The article of manufacture may further comprise a second container comprising a suitable diluent (e.g., BWFI). Upon mixing of the diluent and the lyophilized formulation, the final protein concentration in the reconstituted formulation will generally be at least 50 mg/mL. The article of manufacture may further include other materials desirable from a commercial and user standpoint, including other buffers, diluents, filters, needles, syringes, and package inserts with instructions for use.

### EXAMPLES

The following Examples are not limiting but only exemplary. Unless otherwise specified, all cell lines were purchased from American Type Culture Collection (ATCC, Manassas, VA) and cultured in Dulbecco s Modified Eagle's Medium (DMEM; Thermo Fisher Scientific, Waltham, MA) supplemented with 10% (v/v) Gibco fetal bovine serum (FBS; Thermo Fisher Scientific) at 37° C. in a humidified atmosphere with 5% CO₂. Standard methods were used to manipulate DNA and RNA described in Sambrook et al., Molecular cloning: A laboratory manual; Cold Spring Harbor Laboratory Press, Cold Spring Harbor, New York, 1989. All commercial reagents and kits were used according to the manufacturer's instructions unless otherwise stated.

### Example 1

### Generation of MAb51-31 mAb Specifically Targeting on Human Solid Tumor Cell Lines

### A. Generation of Hybridoma Clones by Live-Cell Immunization and Live-Cell Screening

To obtain mAbs specifically targeting on human malignant tumors, a mixture of three cancer cell lines, AsPC-1, BxPC-3, and Panc10.05, was used for live-cell immunization (LCI) and live-cell high-throughput screening (LC-HTS) as described previously (see: U.S. Pat. No. 11,939,391B2, US20210139602A1, WO2014146487A1, WO2017114204). Hybridoma culture supernatants were screened for binding to the surfaces of these three cell lines and counter-screened with human peripheral blood mononuclear cells (PBMCs) from healthy adult donors (Stanford Blood Center, Palo Alto, CA) using a fluorescence-activated cell sorting (FACS) assay. Briefly, cancer cell lines cultured in DMEM/10% (v/v) FBS were detached using 0.2% (w/v) Ethylenediaminetetraacetic acid (EDTA) and mixed with PBS. The cells were collected by centrifugation at 500×g and blocked with ice-cold PBS containing 1.0% (w/v) bovine serum albumin (BSA; Thermo Fisher Scientific). Hybridoma culture supernatants were incubated with the cells at 4° C. for 30 minutes (min). Surface antibody binding was detected using 1:800 diluted Fluorescein AffiniPure™ Goat Anti-Mouse IgG, Fcγ Fragment Specific (Jackson ImmunoResearch Laboratories, West Grove, PA). After three washes with ice-cold PBS/1.0% (w/v) BSA, cells were fixed with 4.0% (w/v) paraformaldehyde. FACS was performed using BD FACSCalibur™ Flow Cytometer equipped with High Throughput Sampler (HTS), and data were analyzed using FlowJo™ 10.8 software (Becton Dickinson, San Jose, CA).

Hybridoma colonies exhibited high fluorescence signals in binding to the pancreatic cell lines, but very weak or no binding signal to PBMCs in the FACS assay, were selected for expansion, weaning from conditional medium, and subcloning following standard protocols (Kohler & Milstein, Nature 1975, 256:495-497; Winter & Milstein, Nature 1991, 349:293-299). MAbs secreted by these hybridoma subclones were purified from the culture supernatants by affinity chromatography using MabSelect™ SuRe™ LX Protein A resin (Cytiva, Wilmington, DE) and buffer-exchanged into phosphate-buffered saline (PBS, pH7.2; Cytiva). Antibody concentrations were determined by absorbance at 280 nM (A₂₈₀) using a Nanodrop™ 2000 Spectrophotometer (Thermo Fisher Scientific). The purity and integrity of the mAbs were assessed by sodium dodecyl-sulfate polyacrylamide gel electrophoresis (SDS-PAGE) on 4-12% SurePAGE™ Bis-Tris gels (Descript, Piscataway, NJ) stained with SimplyBlue™ SafeStain (Thermo Fisher Scientific). The isotype of each mAb was determined using IsoStrip™ Mouse Monoclonal Antibody Isotyping Kit (Roche Molecular Systems, Pleasanton, CA).

B. FACS Analysis of mAbs in Binding to Cancer Cell Surfaces

The reactivity of purified mAbs was confirmed by assessing their binding to representative cancer cell lines from different cancer types with FACS assays. Purified mAbs, serially diluted from an initial concentration of 10 or 20 μg/mL, were incubated with individual cell lines at 4° C. for 30 min, followed by detection with Fluorescein AffiniPure™ Goat Anti-Mouse IgG, Fcγ Fragment Specific. To evaluate specificity and potential off-target effects, multiple batches of fresh PBMCs from distinct healthy donors were stained in parallel as negative controls. Additionally, frozen human bone marrow mononuclear cells (BMMCs) from healthy donors and human cord blood mononuclear cells (CBMCs) purchased from HumanCells Bioscience (Milpitas, CA) were carefully thawed and stained to assess the safety profile of the mAbs as therapeutic candidates.

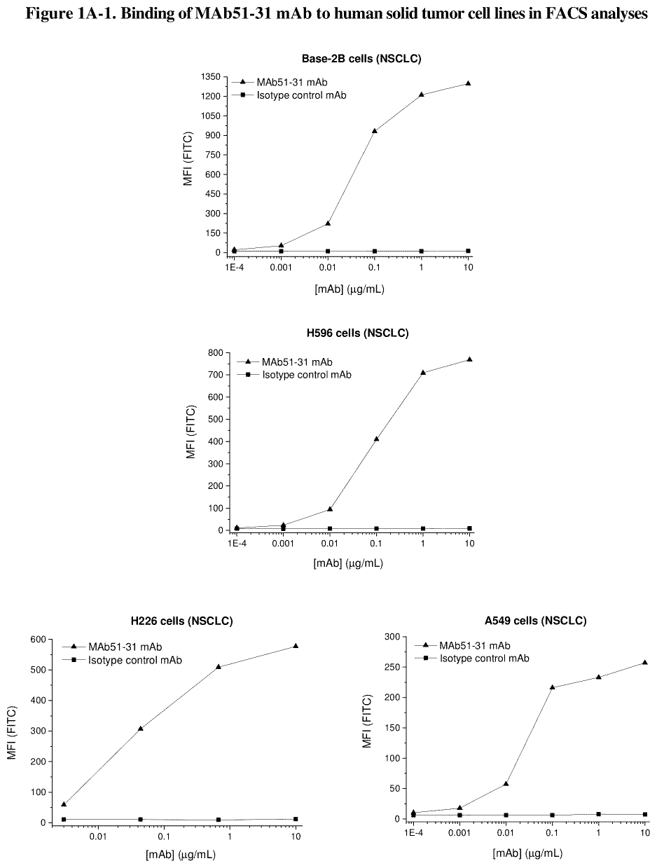

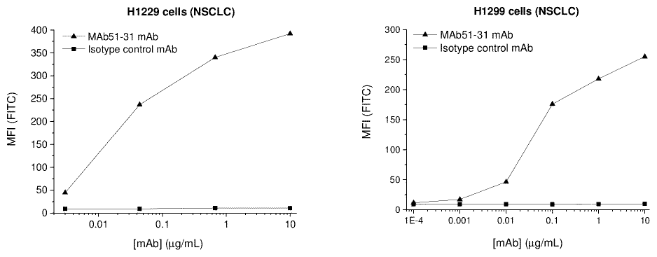

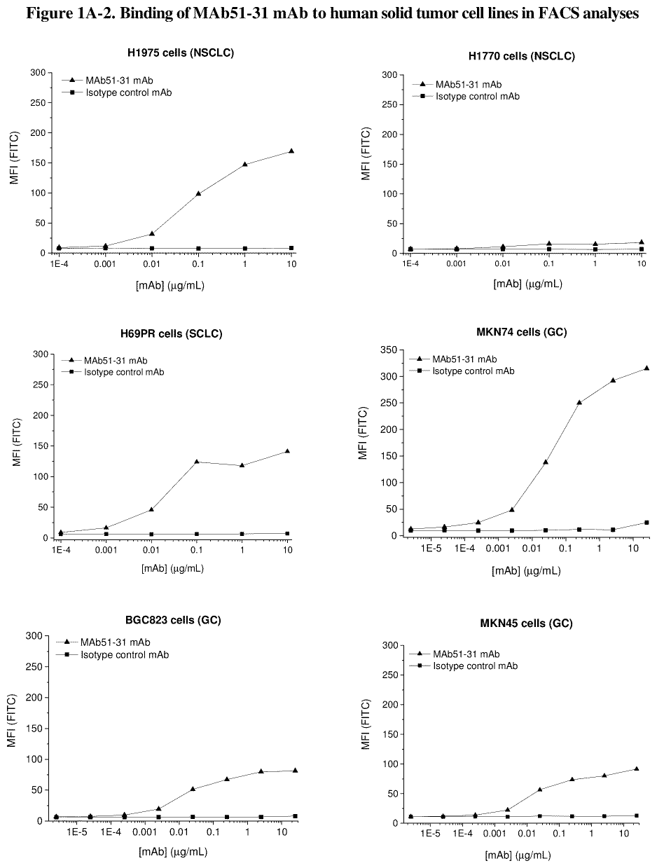

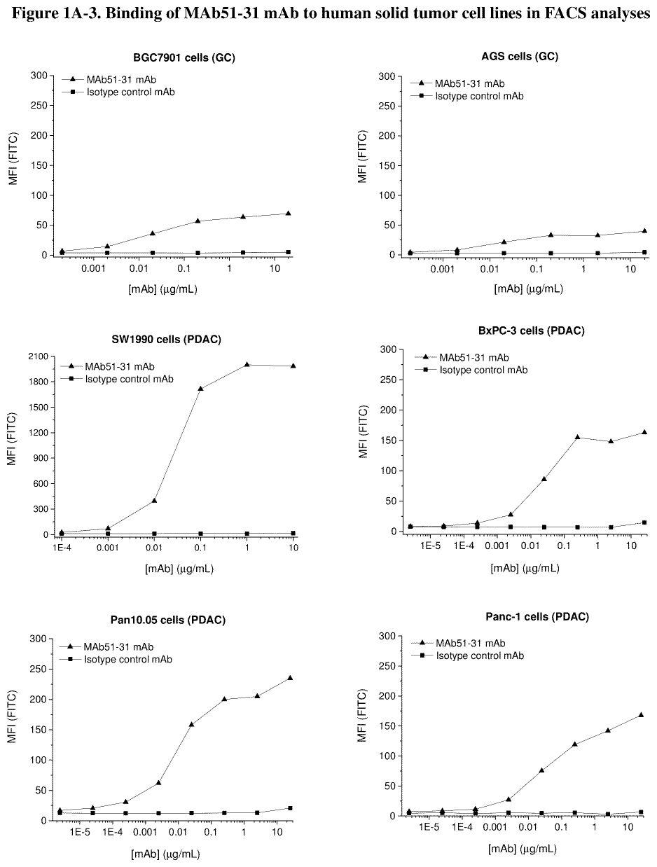

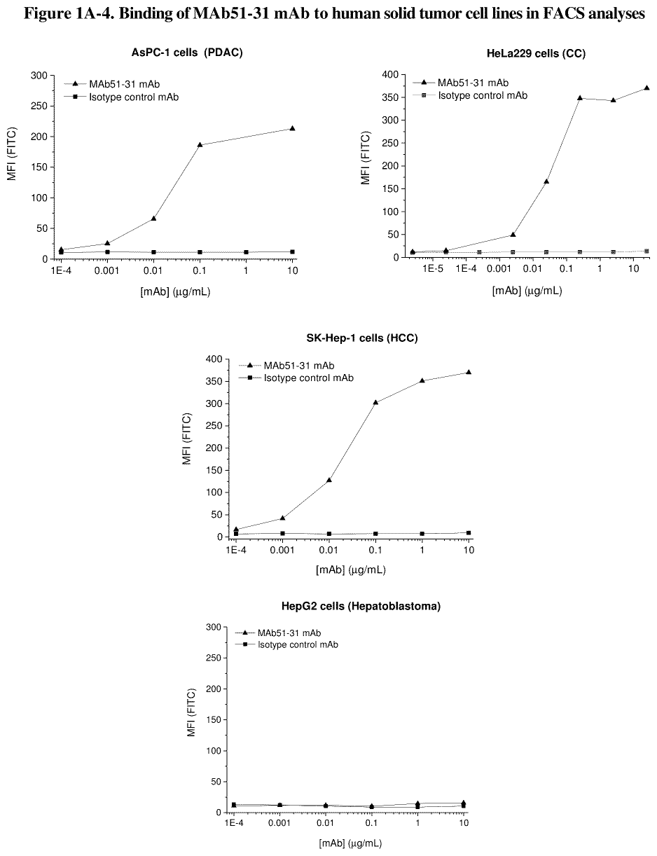

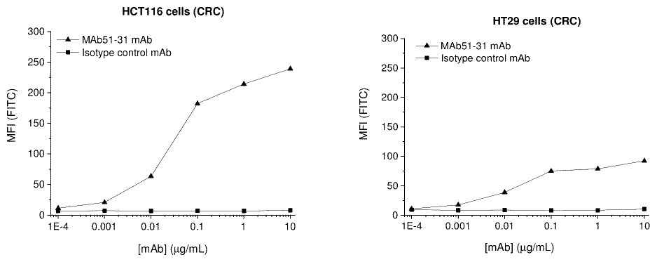

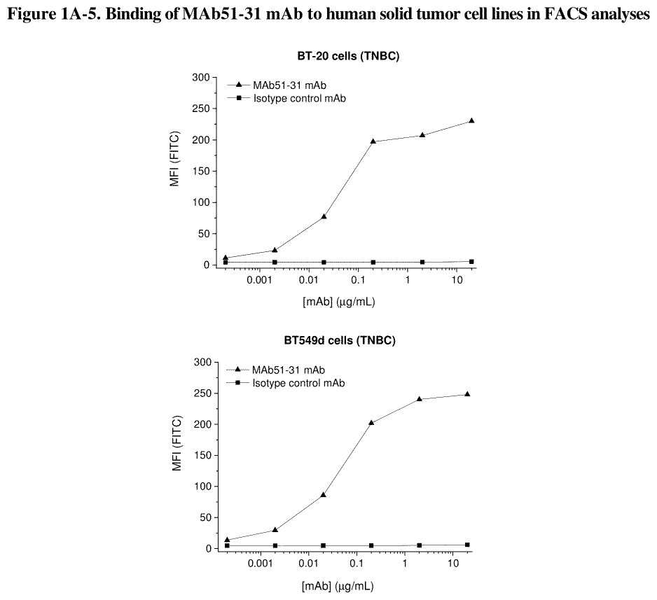

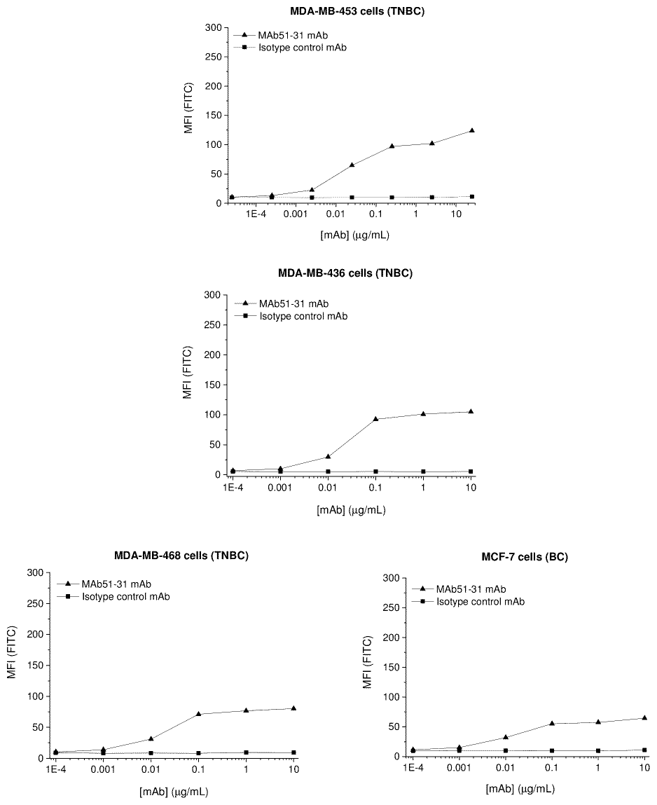

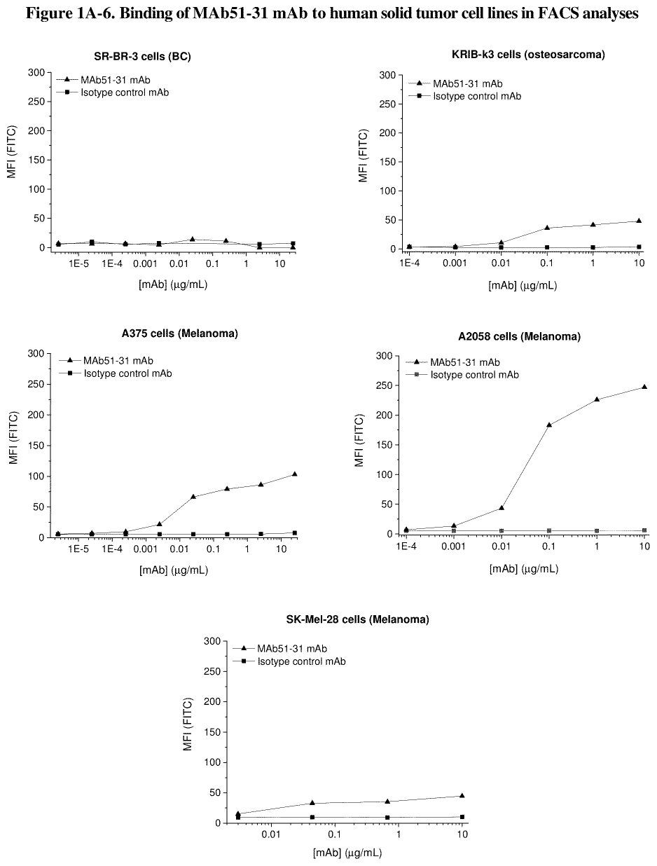

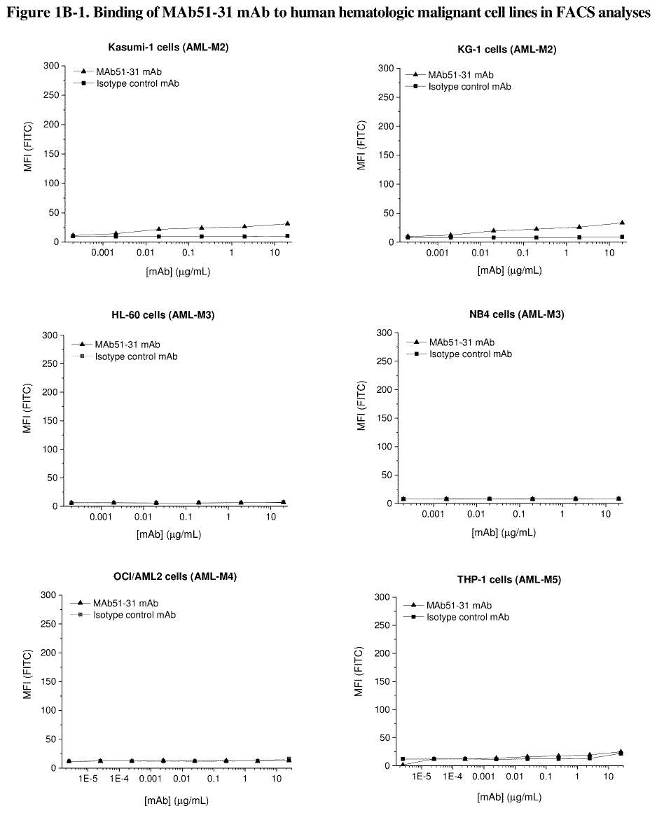

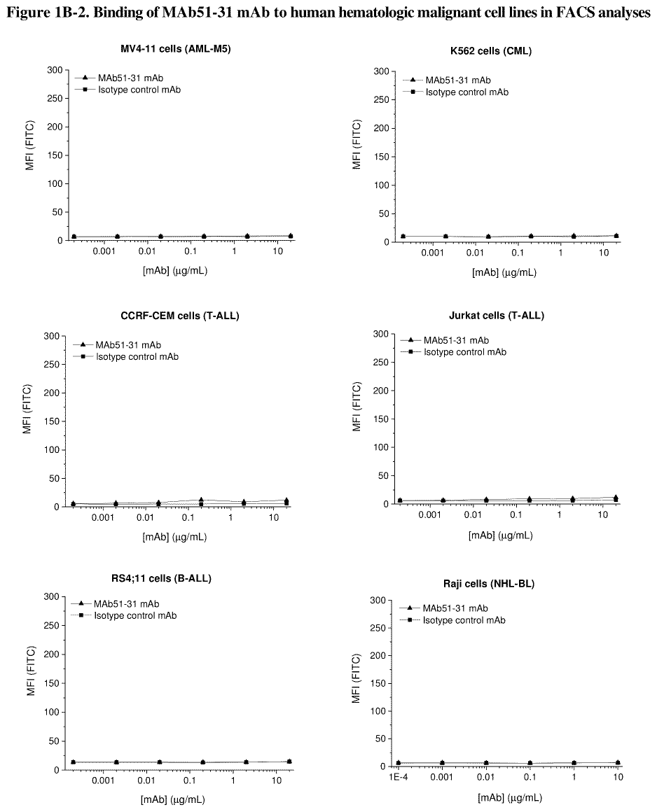

Among all purified mAbs, MAb51-31 mAb exhibited high binding capacity to different types of cancer cells other than pancreatic ductal adenocarcinoma (PDAC). These include non-small cell lung cancer (NSCLC), small cell lung cancer (SCLC), gastric cancer (GC), cervical cancer (CC), hepatocellular carcinoma (HCC), colorectal cancer (CRC), triple-negative breast cancer (TNBC), melanoma, and osteosarcoma (FIG. 1A). On the other hand, only two out of 12 tested cell lines of hematologic malignancy interacted with MAb51-31 mAb (FIG. 1B), implicating that its target is likely more prevalent on solid tumors.

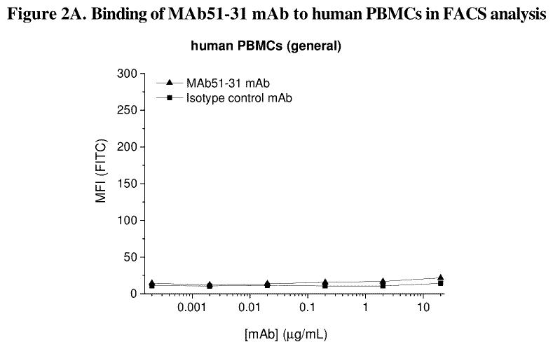

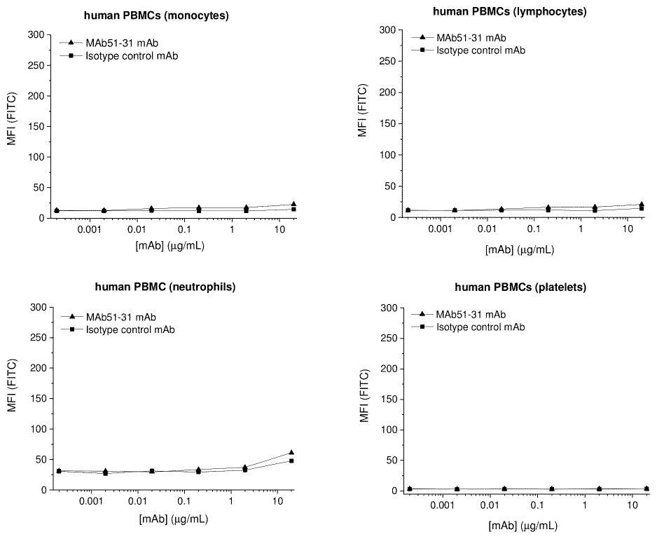

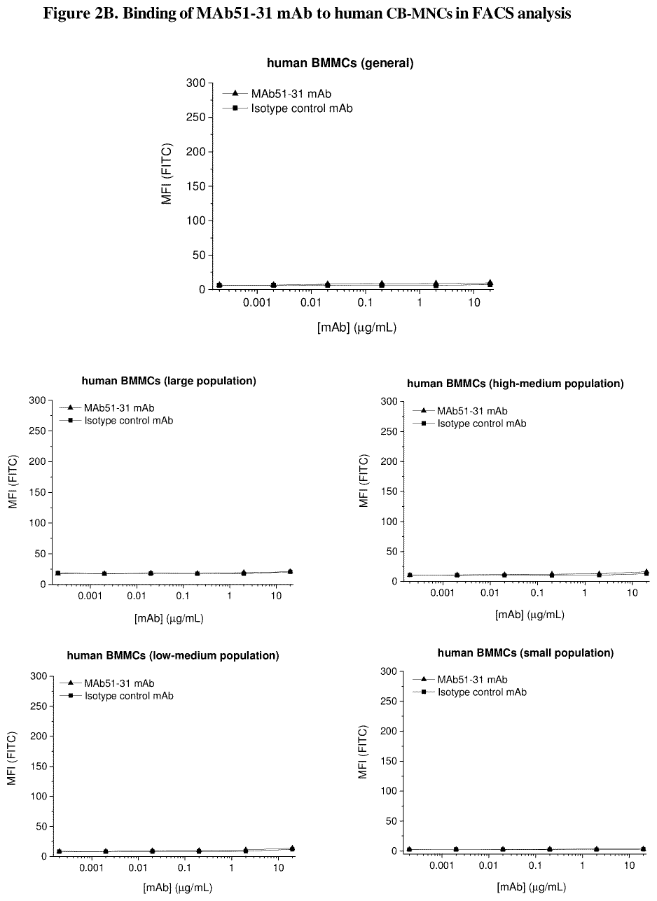

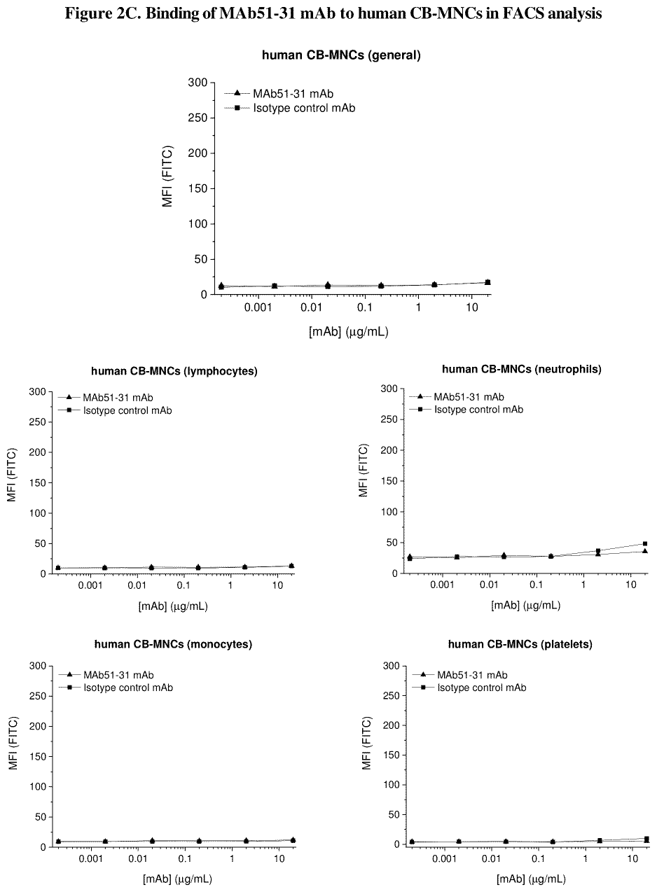

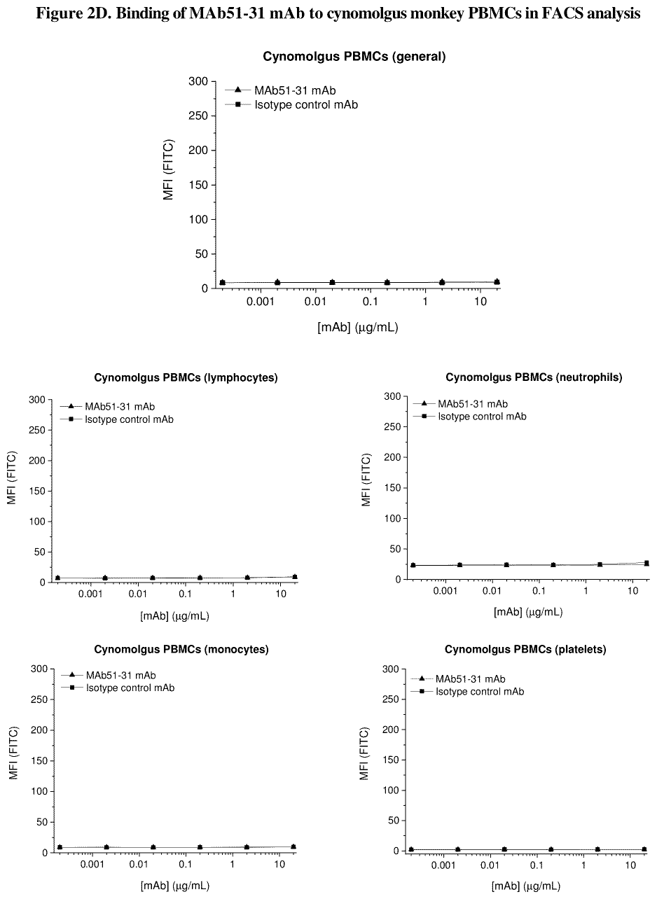

In FACS analyses with normal human cells, MAb51-31 mAb showed no sign of binding to any sub-populations of human PBMCs (FIG. 2A), BMMCs (FIG. 2B), or CBMCs (FIG. 2C). Additionally, it did not cross-react with PBMCs isolated from cynomolgus monkeys (FIG. 2D).

Isotype typing revealed that MAb51-31 mAb consists of a murine kappa light chain and an IgG1 heavy chain, and therefore, a murine IgG1-Kappa isotype control mAb was included in all binding assays for MAb51-31 mAb.

### C. IHC Assay of MAb51-31 mAb in Binding to Normal Human Tissues

To assess whether MAb51-31 mAb interacts with normal human tissues beyond PMBCs, BMMCs, and CBMCs, an immunohistochemistry (IHC) assay was performed using FDA standard formalin-fixed paraffin-embedded (FFPE) tissue microarray (TMA) slides (Biochain, Newark, CA). Each TMA slide contains 30 different human tissue samples, each donated from three distinct donors. Among them, 29 are normal tissues, and the placenta sections serve as a positive control. Lung cancer (NSCLC) FFPE sections were used to validate the IHC protocol and the optimal concentration of MAb51-31 mAb for the assay. Briefly, tissue sections were deparaffinized, rehydrated through graded alcohols, and subjected to heat-induced antigen retrieval (HIER), followed by blocking with 10% (v/v) horse serum (Vector Laboratories, Newark, CA). Sections were incubated overnight at 4° C. with 100 μg/mL MAb51-31 mAb. Antibody binding was detected using VECTASTAIN® Elite® ABC-HRP Kit, Peroxidase (Mouse IgG) from Vector Laboratories. Samples were counterstained with Mayer's hematoxylin solution (MilliporeSigma, St. Louis, MO), mounted in VectaMount® AQ Mounting Medium (Vector Laboratories), and coverslipped. Tissue sections were imaged and scanned using the BZ-X800 All-in-One Fluorescence Microscope (Keyence, Itasca, IL).

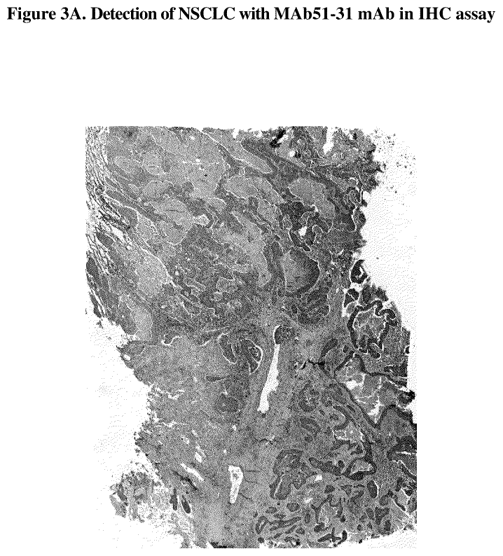

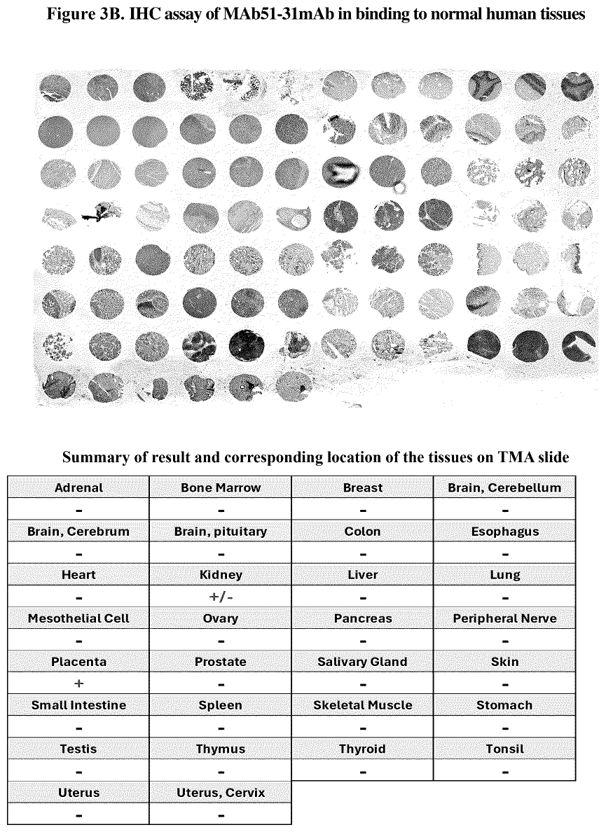

At 100 μg/mL (or 0.67 μM), MAb51-31 mAb effectively stained the lung cancer tissue section (FIG. 3A). Notably, on the normal TMA slide, MAb51-31 mAb showed no staining in 28 of the 29 normal tissue types examined, with only weak interaction observed in the kidney (FIG. 3B). The IHC findings are particularly significant, as they suggest that the target of MAb51-31 mAb is cancer-specific and absent in normal tissues, highlighting its potential as a promising candidate for both therapeutic and diagnostic applications.

### Example 2

### Identification and Verification of Human ITGα3/β1 as the Target of MAb51-31 mAb

### A. Target Identification by Immunoprecipitation (IP) and Mass Spectrometry (MS)

To identify the target of MAb51-31 mAb, it was conjugated to Dynabeads™ Protein-A (Thermo Fisher Scientific) for immunoprecipitation, or to horse radish peroxidase (HRP) with Lightning-Link® HRP Conjugation Kit (Abcam, Waltham, MA), according to the manufacturers' protocols. Cancer cell lines that showed strong reactivity with MAb51-31 mAb were lysed with radioimmunoprecipitation assay (RIPA) buffer (Thermo Fisher Scientific), and the lysates of mixed cancer cell lines were incubated with the Dynabeads™-MAb51-31 mAb complex at room temperature (RT) for 30 min. The complex was washed sequentially with 0.5% (v/v) TritonX-100/PBS and PBS. The immunoprecipitated (IP) protein samples were separated by SDS-PAGE and transferred to a nitrocellulose membrane (MilliporeSigma). The membrane was blocked with PBS containing 0.05% (v/v) Tween®-20 (PBST) and 1% (w/v) BSA and probed with HRP-conjugated MAb51-31 mAb, followed by detection using Suat erSignal™ West Pico PLUS Chemiluminescent Substrate (Thermo Fisher Scientific).

However, HRP-conjugated MAb51-31 mAb failed to detect any specific bands in the Western blot analysis, suggesting that the target(s) is likely a conformational epitope that was denatured during the Western blot process. This indicates that the antibody recognizes a three-dimensional structure rather than a linear peptide sequence. To further investigate, the MAb51-31 mAb IP samples were digested directly with trypsin and Lys-C protease, and analyzed by liquid chromatography with tandem mass spectrometry (LC-MS/MS, Innomics, San Jose, CA). The resulting data were searched against the latest reviewed human database from Uniprot (https://www.uniprot.org/) using Mascot.

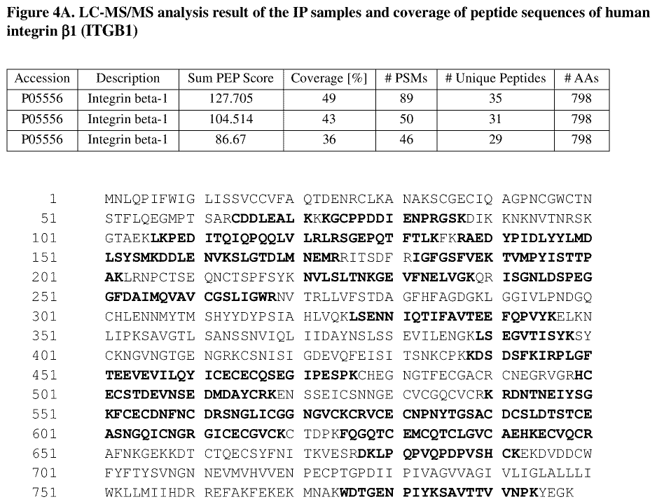

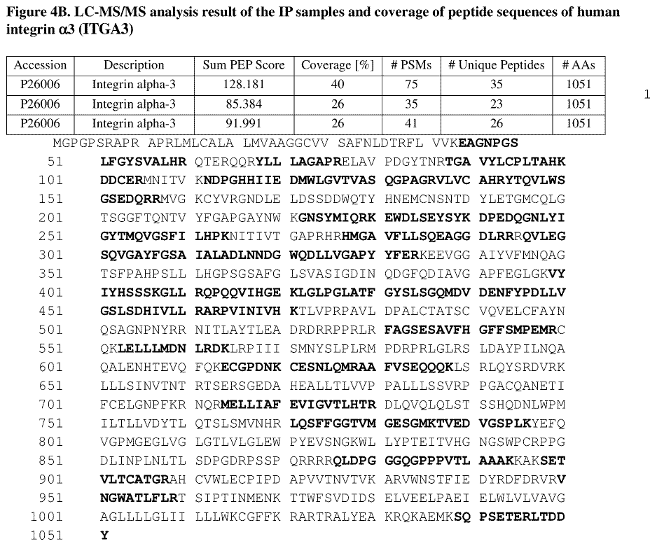

The most confidently identified proteins detected by IP and LC-MS/MS analyses were integrin β1 (ITGB1, UniProtKB/Swiss-Prot: P05556) and integrin α3 (ITGA3, UniProtKB/Swiss-Prot: P26006), with 49% and 40% sequence coverages, respectively (FIGS. 4A and 4B). Although ITGB1 had a higher score than ITGA3, because ITGA3 exclusively pairs with ITGB1, whereas ITGB1 can pair with 12 different α subunits, and no other integrin a subunits were detected in the IP samples, we propose that MAb51-31 mAb specifically binds to a conformational epitope within the ITGα3/β1 heterodimer, which depends on the structural change induced by the interaction between ITGA3 and ITGB1. This hypothesis was further evaluated through ELISA assays as followed. Other proteins identified showed much lower scores and were excluded by ELISA assays (data not shown).

### B. Verification of Interaction Between MAb51-31 mAb and ITGα3/β1 by ELISA Assay

ELISA assays using different integrin complexes or subunits were performed to confirm the direct interaction between MAb51-31 mAb and human ITGα3/β1, and to specify which subunit is involved in the interaction. Two versions of recombinantly expressed ITGα3/β1 heterodimers comprising the extracellular domains (ECDs) of ITGA3 (Phe33-Glu991) and ITGB1 (Glu21-Asp728), designated as rhITGα3/β1 and rhITGα3/β1-His, were purchased from R&D Systems (Minneapolis, MN) and AcroBiosystems (Newark, DE), respectively, for cross-verification. These two protein complexes differ in the presence of a C-terminal His-tag in ITGA3 as indicated. Two recombinant ITGB1-ECD (Gln21-Asp728) containing a C-terminal His tag (rhITGB1-ECD-His) expressed in HEK293 cells were from Novus Biologicals (Centennial, CO) and SinoBiological (Wayne, PA). Recombinant full-length (FL) ITGA3 with a C-terminal FLAG tag (rhITGA3-FLAG) was from Origene Technologies (Rockville, MD).

These recombinant proteins were individually immobilized on Immulon® microtiter plates (Thermo Fisher Scientific) at 0.15 μg/mL of concentration, washed with PBST and blocked with PBST/1% (w/v) BSA at RT for one hour. MAb51-31 mAb, anti-6×His-tag mAb (Proteintech, Rosemont, IL), or isotype control mAb was added in serial dilutions and incubated for one hour. After washing, the plates were probed with 1:4,000 diluted Peroxidase AffiniPure™ Goat Anti-Mouse IgG, Fcγ fragment specific (Jackson ImmunoResearch Laboratories) for 30-60 min. Following thorough wash, 3,3′,5,5′-Tetramethylbenzidine (TMB) substrate was added to allow color development for 15 min, and then stopped with 0.05 M sulfuric acid. Absorbance at 450 nm (A₄₅₀) was measured using the SpectraMax Microplate Reader (Molecular Device, San Jose, CA).

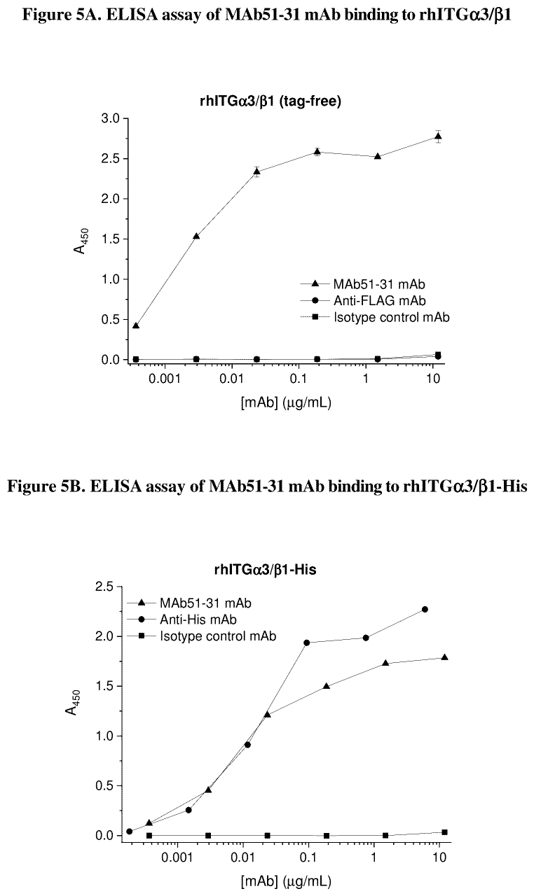

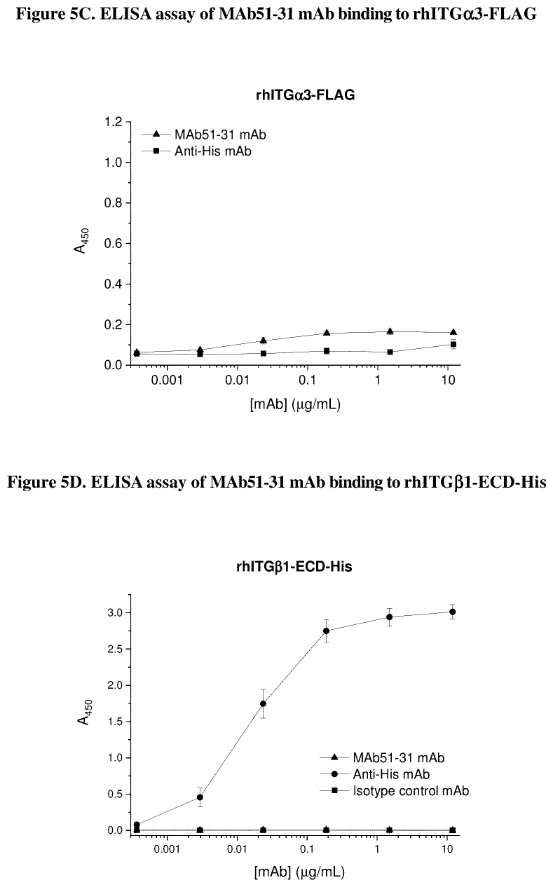

MAb51-31 mAb bound with a high affinity to both rhITGα3/β1 (FIG. 5A) and rhITGα3/β1-His (FIG. 5B) heterodimers in a dose-dependent manner, confirming the LC-MS/MS findings from the IP samples that ITGα3/β1 was the potential target. However, it showed very low binding affinity for the rhITGA3-FLAG monomer (FIG. 5C) and no detectable interaction with the rhITGB1-ECD-His monomer (FIG. 5D). Although no direct contact between MAb51-31 mAb and ITGB1 was observed, the substantial difference in binding between the recombinant ITGα3/1 heterodimers and the ITGA3 monomer suggests that ITGB1 is essential for the robust binding of MAb51-31 mAb to ITGα3/β1 heterodimeric complex as well as to cancer cells. Thus, MAb51-31 mAb likely recognizes a conformational epitope located on the ITGα3/β1 heterodimer that is not present in isolated ITGA3 or ITGB1 subunits, but rather emerges upon their assembly. This explains why MAb51-31 mAb failed to detect any specific bands in the IP samples during Western blot analysis, as the quaternary structure of the ITGα3/β1 duplex was disrupted in polyacrylamide gel containing SDS.

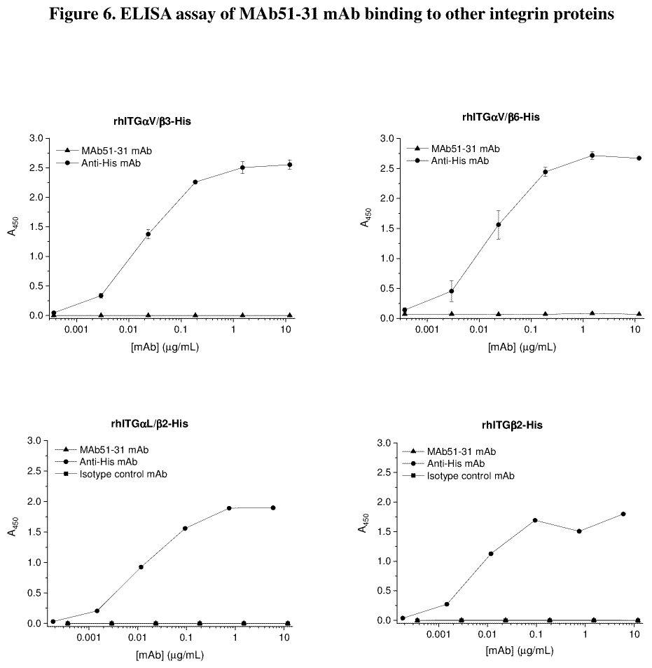

Given that different integrin subunits share sequence similarities and dimeric complexes may exhibit common structural features, we performed ELISA assays on additional integrin proteins, including rhITGB2, rhITGαL/β2, rhITGαV/β3, and rhITGαV/B6, to assess potential cross-reactivity of MAb51-31 mAb. As shown in FIG. 6, MAb51-31 mAb did not bind to any of these tested integrins.

Additionally, an ELISA competition assay showed that the binding of MAb51-31 mAb to rhITGα3/β1 was not affected by iMatrix-332, a recombinant human laminin 332-E8 fragments protein known to be a ligand for ITGα3/β1 and ITGα6/4 (data not shown). This suggests that conformational epitope recognized by MAb51-31 mAb does not overlap with the laminin binding site.

C. Binding Kinetics and Affinity Between MAb51-31 mAb and rhITGα3/β1-his Determined by Octet

The binding kinetics and affinity of MAb51-31 mAb to the rhITGα3/1-His complex was determined using Bio-Layer Interferometry (BLI) label-free technology with an Octet® QK^{e} System (Molecular Devices, San Jose, CA). Anti-Mouse IgG Fc Capture (AMC) Biosensors (Sartorius, Fremont, CA) were immobilized with 50 nM MAb51-31 mAb for 10 min, followed by a 5-min wash with 1× Kinetic Buffer (Sartorius). After the baseline stabilized, sensors loaded with MAb51-31 mAb were exposed to rhITGα3/β1-His for 15 min to obtain the association curve, followed by 30 min of dissociation in 1× Kinetic Buffer. The assay was repeated multiple times using 2-fold serial dilutions of rhITGα3/β1-His, ranging from 100 nM to 12.5 nM, in 1× Kinetic Buffer. Kinetic data were processed with average referencing and fit to a 1:1 interaction model using Octet® Data Analysis software (version 10) to determine the association rate constant (the on-rate or kₒₙ) and dissociation rate constant (the off-rate or k_{off}). The equilibrium dissociation constant (K_{D}) in molar units (M) was calculated using the formula K_{D}=K_{off}/kₒₙ. The biosensors were regenerated with 10 mM glycine, pH 1.70.

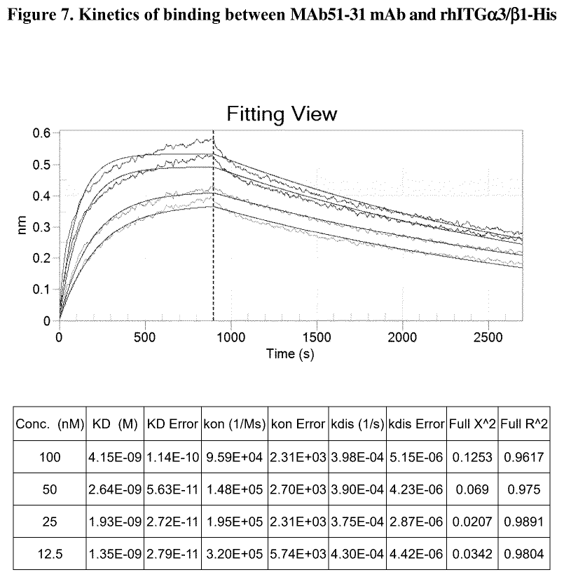

The raw association and dissociation curves between MAb51-31 mAb and rhITG&3/β1-His are shown in FIG. 7. A fast on-rate and a moderate off-rate suggests a relatively high affinity. The affinity (K_{D}) calculated was approximately 2.5×10⁻⁹ M.

D. Detection of ITGA3 Gene Expression in Cancer Cell Lines by qRT-PCR

MAb51-31 mAb exhibited strong binding signals to various cancer cell lines (FIG. 1A), suggesting that the ITGα3/β1 complex with aberrant conformational epitope is highly expressed on cell surfaces. Since ITGB1 is involved in forming many other integrin heterodimers while ITGα3 exclusively complexes with ITGB1, we only measured the relative expression levels of the ITGA3 gene in cancer cell lines using quantitative reverse transcription-polymerase chain reaction (qRT-PCR). A375 (melanoma), A549 (NSCLC), BT549d (TNBC), HCT-116 (CRC), MKN45 (GC; AffiGen, Baileys Harbor, WI), and Panc-1 (PDAC) cell lines were cultured in DMEM supplemented with 10% (v/v) FBS until reaching exponential phase. The cells were detached, collected by centrifugation, and washed once with PBS before proceeding to RNA isolation. Human PBMCs from healthy adult donors were used as the baseline control.

Total RNA was isolated from the cells using Quick-RNA™ Miniprep Kit (Zymo Research, Irvine, CA), and residual genomic DNA (gDNA) was removed by DNase I digestion (Thermo Fisher Scientific) according to the manufacturer's protocol. First-strand cDNA was synthesized at 25° C. for 10 min, 37° C. for 120 min, and 85° C. for 5 min using the High Capacity cDNA Reverse Transcription Kits (Thermo Fisher Scientific) in a 20-μL reaction containing 1 μg of total RNA, 2 μL of 10×RT buffer, 4 mM dNTP mix, 2 μL of RT Random Primers, 1 μL of MultiScribe™ Reverse Transcriptase, and 1 μL of RNase Inhibitor. Real-time PCR was performed on the ABI7800 Real Time PCR System (Thermo Fisher Scientific) using Luna Universal qPCR Master Mix (New England Biolabs, Ipswich, MA) in a 20-μL reaction with 0.2 μL of first-strand cDNA as the template. The cycling conditions were as follows: 95° C. for 60 sec, followed by 40 cycles of 95° C. for 15 sec and 60° C. for 30 sec. Relative mRNA expression levels were calculated using the comparative cycle threshold (2^{-ΔΔCT}) method (Livak and Schmittgen, Methods 2001). Ribosomal protein lateral stalk subunit P0 (RPLP0) served as the housekeeping control. The primer sequences for qPCR were:

ITGA3 Forward:
(SEQ ID NO: 20)
5′-TCACCAACGTGACTGTGAAG-3′
ITGA3 Reverse:
(SEQ ID NO: 21)
5′-GATGCTGGTTCGGAGGAATAG-3′
RPLP0 Forward:
(SEQ ID NO: 22)
5′-GGCGACCTGGAAGTCCAACT-3′
RPLP0 Reverse:
(SEQ ID NO: 23)
5′-CCATCAGCACCACAGCCTTC-3′

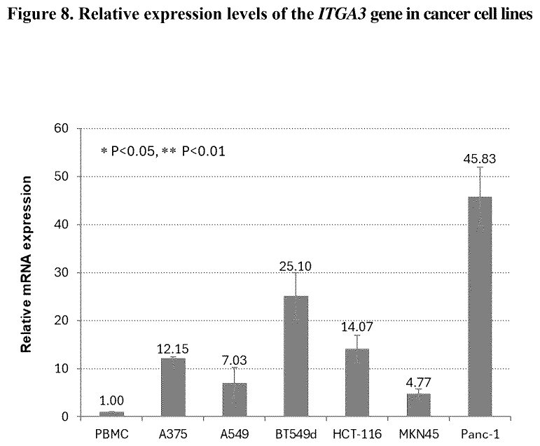

The qRT-PCR result revealed that the ITGA3 gene was significantly upregulated in all six cancer cell lines checked, with 4.8 to 45.8-fold increase compared with PBMCs, which express minimal level of ITGA3 (FIG. 8). Although the relative expression levels of the ITGA3 gene and the ITGα3/β1 protein (FIG. 1A) were inconsistent in tested cancer cell lines likely due to mRNA stability, translation regulation, post-translational modification (PTM), or protein degradation, overexpression of ITGA3 at both gene and protein levels supports its potential role in tumor invasion and metastasis.

### Example 3

### In Vitro Effects of MAb51-31 mAb on Cancer Cell Lines

### A. Cell Invasion Assay of MAb51-31 mAb

Anti-integrin antibodies have been reported to regulate the growth or proliferation of cells through various mechanisms, including stimulatory or inhibitory effects, while some exhibit no activity upon binding to integrins (Byron et al., 2009). We conducted a cell proliferation assay (see protocol in Example 7) but observed no inhibition of cell growth across multiple cancer cell lines treated with MAb51-31 mAb (data not shown). The QCM ECMatrix Cell Invasion Assay Kit (MilliporeSigma) was used to assess whether MAb51-31 mAb interferes with cancer cell invasion. A375, A549, BT549d, HCT-116, MKN45, and Panc-1 cell lines were seeded at a density of 3×10⁵ cells/well in serum-free DMEM supplemented with 1% (w/v) BSA into the 24-well plate provided by the kit. The cells were treated with 150 μg/mL of MAb51-31 mAb or an isotype control mAb and incubated overnight at 37° C. with 5% CO₂ and humidity. Invasive cells that migrated through the ECM layer were stained according to the manufacturer's instructions and quantified by measuring absorbance at 560 nm (A₅₆₀) using a microplate reader. Each sample was measured in triplicates.

For each cell line, the difference in number of cells that invaded through the ECM matrix between the MAb51-31 mAb-treated group (test) and isotype control mAb-treated group (control) was calculated the following formula:

$$Percent change in invasive cells ( \% ) = [ ( {A}_{560} test - {A}_{560} control ) / {A}_{560} control ] \times 100.$$

Isotype control samples were used as the baseline, represented as 0±standard deviation (SD) of triplicated samples. A positive result indicates an increase in cell invasion, whereas a negative result signifies a percentage of reduction in cell invasion.

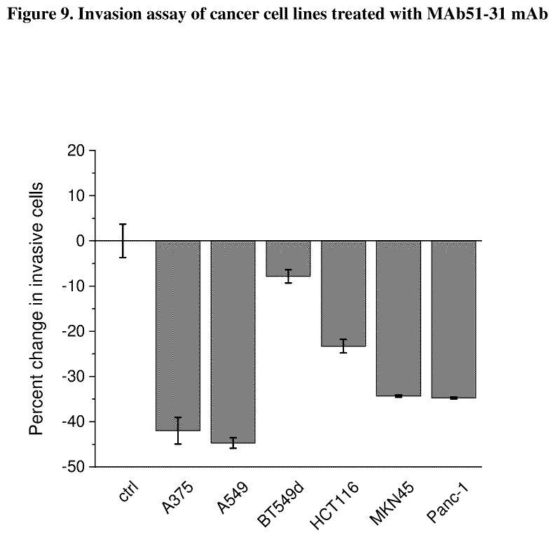

After 24 hours of co-incubation with MAb51-31 mAb, A375, A549, HCT-116, MKN45, and Panc-1 cells exhibited a significant reduction in the number of cells that migrated across the ECMatrix™ membrane (FIG. 9). This suggests that MAb51-31 mAb has an inhibitory effect on cell invasion. Although MAb51-31 mAb did not demonstrate cytotoxic effect on cancer cells, it may partially inhibit cancer metastasis.

### B. Internalization Assay of MAb51-31 mAb by Cancer Cell Lines

MAb51-31 mAb exhibits an affinity (~2.5 nM) for human ITGα3/β1, recognizing a conformational epitope that relies on the interaction between ITGA3 and ITGB1. Although the antibody itself does not directly inhibit cancer cell growth, its strong affinity and high specificity make it a promising candidate for ADC development. Internalization of ITGα3/β1 is not well studied. Recently, Cegarra et al. reported that an anti-ITGα3/1 and ITGα5/B1 mAb, 4F2, could be internalized by brain cells and execute transcytosis across the blood-brain barrier (BBB). Therefore, an internalization assay using FITC-conjugated MAb51-31 mAb to assess whether our anti-ITGα3/β1 mAb could be internalized upon binding to ITGα3/β1, was performed.

Purified MAb51-31 mAb and the isotype control mAb were first labeled with fluorescein (FITC) using Abcam's FITC Conjugation Kit (Abcam). This was done by incubating the mAbs with Modifier Reagent and FITC Mix RT for 3 hours, followed by quenching of any unbound FITC. Cell lines were cultured in DMEM medium supplemented with 10% (v/v) FBS, and plated at optimal densities ranging from 1 to 5×10⁴ cells per well in four Falcon flat-bottomed 96-well tissue culture microplates (Corning, Corning, NY). The cells were incubated overnight at 37° C. with 5% CO₂ and humidity. The cells were pre-cooled to 4° C. and then co-incubated with 0.1 μg/mL FITC-conjugated MAb51-31 mAb (MAb51-31mAb-FITC), or isotype control mAb-FITC at 4° C. to allow surface binding of the mAb-FITC. One hour later, the cells were rinsed twice with ice-cold PBS to remove the unbound mAb-FITC. One plate was subsequently stained for 15 min at 4° C. with 4′,6-Diamidino-2-Phenylindole, dihydrochloride (DAPI; Biotium, Fremont, CA), rinsed with ice-cold PBS, and then photographed using the BZ-X800 All-in-one Fluorescence Microscope (Keyence, Itasca, IL). Meanwhile, warm culture medium was added to the remaining three plates and incubated at 37° C. for 15 min, 30 min, 2 hours, respectively, to promote internalization of the mAb-FITC and antigen complexes. These plates were also stained with DAPI for 15 min at 37° C. prior to PBS wash and imaging.

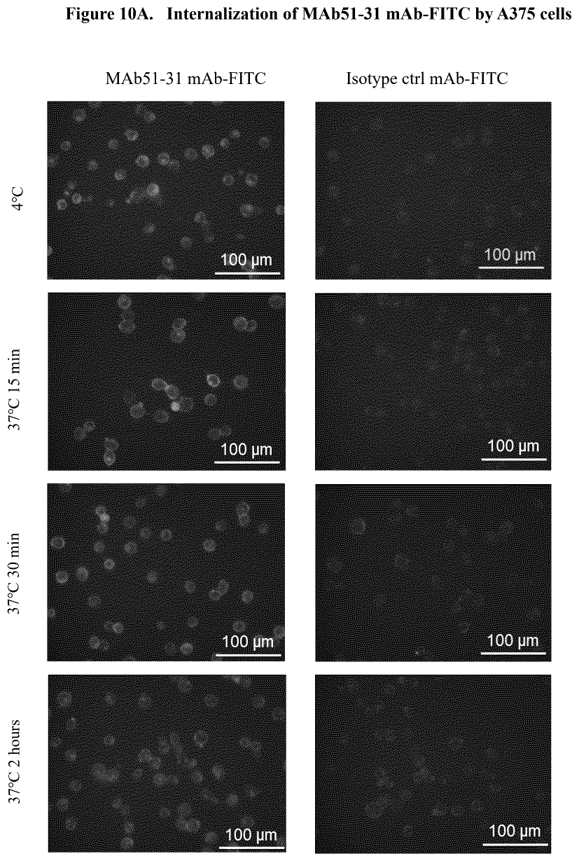

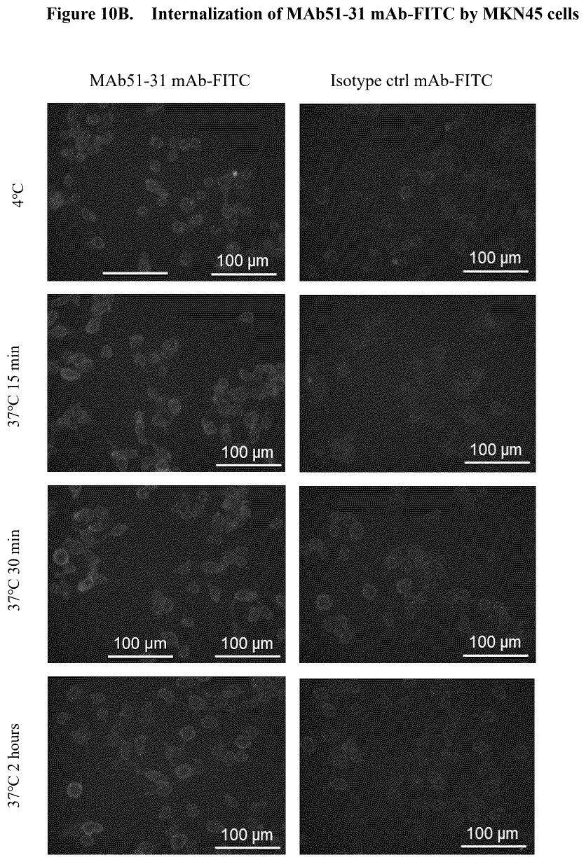

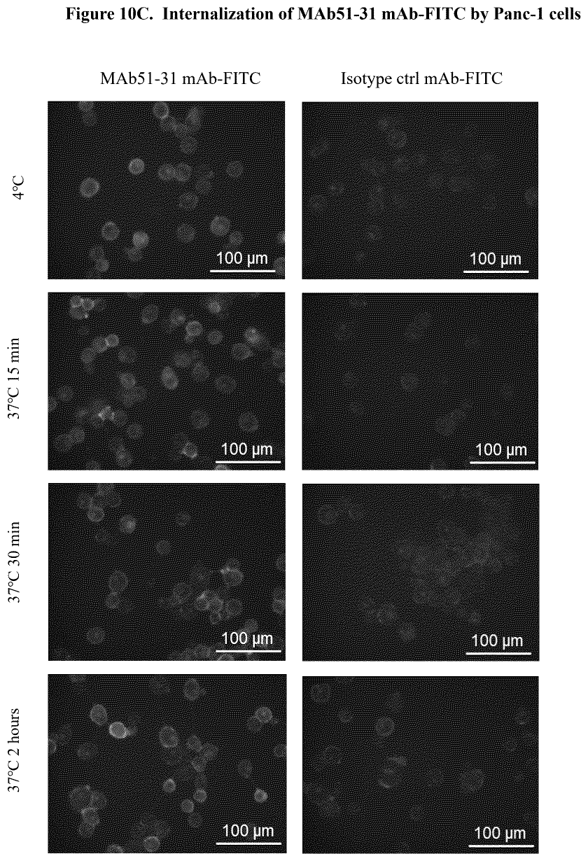

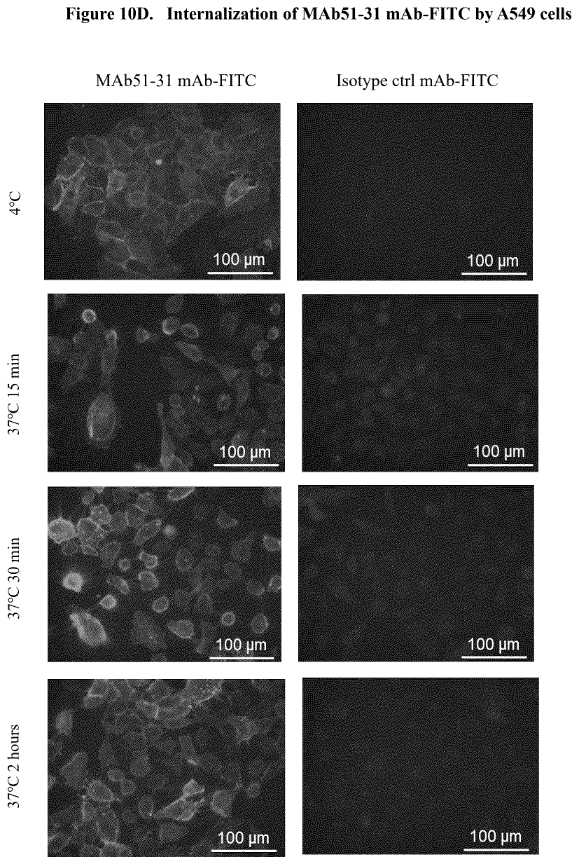

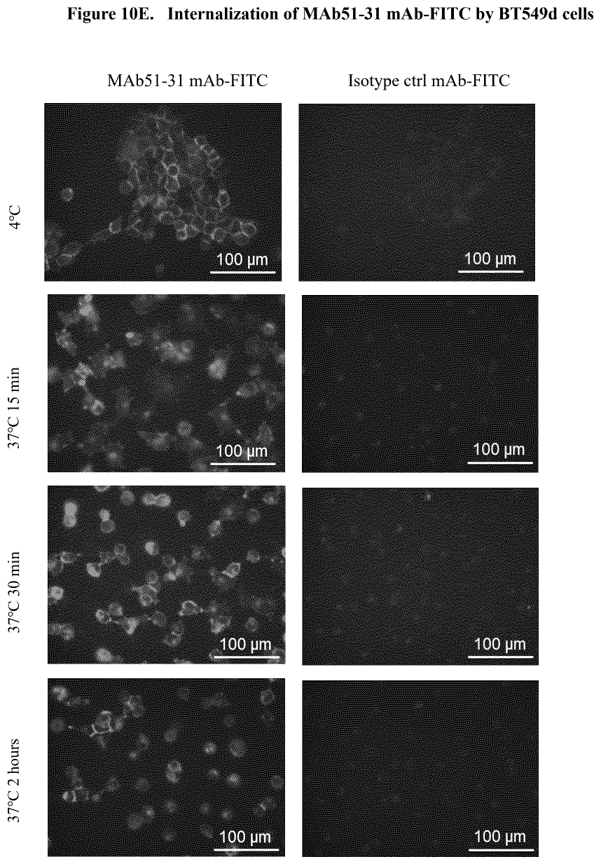

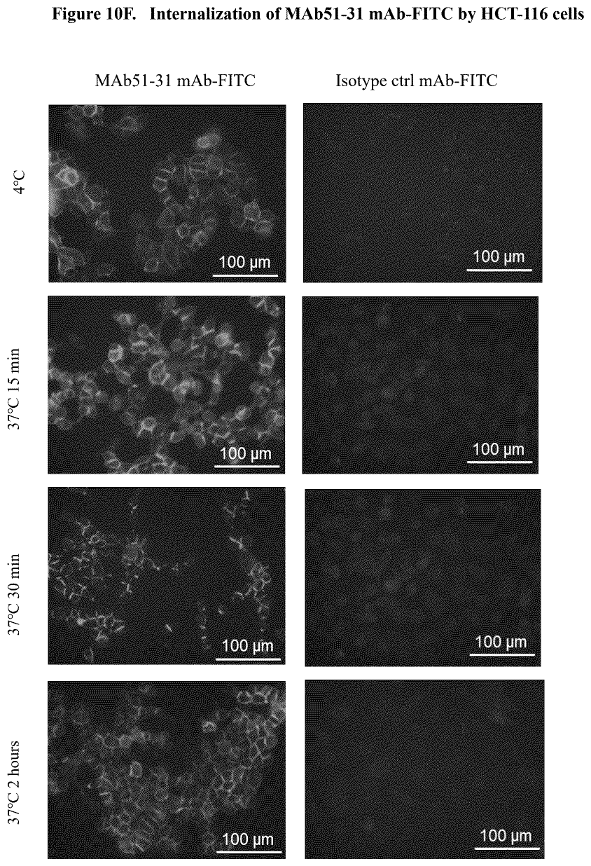

As anticipated, no fluorescence was observed in any of the cancer cell lines treated with the isotype control mAb-FITC (FIGS. 10A to 10G, right panels). MAb51-31mAb-FITC exhibited green fluorescence outlining the cell membrane after one hour of incubation at 4° C., indicating binding to its target on the cell surface (FIGS. 10A-10G, left panels). The intensity of surface binding fluorescence varied between cell lines, with two gastric cancer cell lines, BGC823 (FIG. 10G) and MKN45 (FIG. 10B), much weaker than the other cell lines, correlating with the cell staining results as shown in FIG. 1A. Notably, fluorescence accumulated in the cytoplasm of A375 (FIG. 10A), MKN45 (FIG. 10B), and Panc-1 (FIG. 10C) cells after one-hour incubation at 4° C., suggesting partial internalization of MAb51-31mAb-FITC despite the low cell membrane fluidity at 4° C. However, as incubation continued at 37° C. from 15 min to 2 hours, no significant change in membrane-bound and cytoplasmic fluorescence was observed with these cells. In contrast, A549 (FIG. 10D) and BT549d cells (FIG. 10E) exhibited intracellular fluorescence 15 min after incubation at 37° C. and fluorescence continue to accumulate over the incubation time course at 37° C. By 2 hours, the internalized MAb51-31mAb-FITC account for nearly 50% of the total bound mAb. In contrast, HCT-116 cells, though had strong surface interaction with MAb51-31 mAb-FITC, showed limited amount of MAb51-31 mAb-FITC being engulfed (FIG. 10F).

Differences in internalization efficiency may be influenced not only by antigen expression levels but also by intrinsic turnover rates of ITGα3/β1 and post-translational modification (PTM) such as glycosylation, phosphorylation, and ubiquitination. These studies demonstrate efficient and robust internalization of MAb51-31 mAb across various cancer types, including A375, A549, BT549d, and Panc-1, highlighting its potential as an ADC candidate for multi-cancer treatment. Additionally, evaluating its transcytosis capability in future studies could provide valuable insights into its potential for drug delivery across BBB against brain malignant tumors.

### Example 4

Molecular Cloning of the Light and Heavy Chain Genes Encoding MAb51-31 mAb and Expression of Chimeric Antibody (cAb)

### A. Cloning and Sequencing of the Light and Heavy Chain Genes by 5′-RACE

The cDNA sequences encoding the variable light (VL) and variable heavy (VH) regions of the MAb51-31 hybridoma were determined using 5′-Rapid Amplification of cDNA Ends (5′-RACE). Total RNA was extracted from the MAb51-31 hybridoma cells using the Quick-RNA™ Miniprep Kit (Zymo Research). Reverse transcription and first-strand cDNA synthesis were performed using the SMARTer RACE 5′/3′ Kit (Takara Bio USA, San Jose, CA). The resulting cDNAs was then subjected to 5′-RACE PCR amplification using the following reverse primers to pair with the common forward primer, Universal Primer A Mix (UPM), provided by the kit.

Light chain reverse primer:
(SEQ ID NO: 24)
5′-CTGCTCACTGGATGGTGGGAAGATGG-3′;
Heavy chain reverse primer:
(SEQ ID NO: 25)
5′-AGCTGGGAAGGTGTGCACAC-3′;

The amplified PCR products were cloned into the pRACE vector (Takara) and transformed into Stellar™ E. coli competent cells (Takara) under ampicillin selection. Ten E. coli colonies containing VL and VH DNA fragments were randomly selected for plasmid purification using Hi-Speed Mini Plasmid Kits (IBI Scientific, Dubuque, Iowa). The purified plasmids were subjected to Sanger sequencing (Molecular Cloning Laboratories, South San Francisco, CA), and all sequences were analyzed through BLAST (Basic Local Alignment Search Tool) search against public databases, including NCBI IgBlast (https://www.ncbi.nlm.nih.gov/igblast/), the international ImMunoGeneTics (IMGT) information System®(http://www.imgt.org/IMGT_vquest/), and Uniprot (https://www.uniprot.org/blast), for the novelty of the V domains. Consensus sequences of VL and VH of MAb51-31 mAb are shown in the SUMMARY section as SEQ ID NO: 1 and SEQ ID NO: 3, respectively. The encoded amino acid sequences are shown as SEQ ID NO: 2 and SEQ ID NO: 4, respectively.

### B. Construction of Expression Plasmid for MAb51-31cAbm2

The VL and VH genes of MAb51-31 mAb were amplified via PCR using Q5® High-Fidelity DNA Polymerase (New England Biolabs) from the sequencing plasmids using the cloning primer pairs shown as SEQ ID NO: 26 to 29 below.

VL Cloning Forward primer:
(SEQ ID NO: 26)
5′-AGCACTAGTGCCGCCACCATGGATTTTCAAGTGCAG-3′;
VL Cloning Reverse primer:
(SEQ ID NO: 27)
5′-CTGGGCGCCGCTACAGTCCGTTTGATTTCCAGCGTGGTGC-3′;
VH Cloning Forward primer:
(SEQ ID NO: 28)
5′-AGCTCTAGAGCCGCCACCATGAAATGCAGCTGGGTTATC-3′;
VH Cloning Reverse primer:
(SEQ ID NO: 29)
5′-CTTTGGTGCTAGCTGCAGAGACTGTGAGAGTGGTGCC-3′

The PCR products were gel-purified using the Monarch DNA Gel Extraction Kit (New England Biolabs) and cloned in-frame into the pCMV-hIgG1K-m2 vector with NEBuilder® HiFi DNA Assembly Cloning Kit (New England Biolabs), following the manufacturer's protocol. The pCMV-hIgG1K-m2 vector encodes the human kappa light chain constant region (CL) and the human IgG1 heavy chain constant region (CH1-CH3), under the control of the murine CMV promoter and the human CMV IE1 promoter, respectively. Additionally, each hIgG1 constant region contains two cysteine (Cys of C) point mutations, enabling rapid screening of ADC candidates via site-specific conjugation to various payloads and linkers. Upon in-frame fusion with the murine MAb51-31 VL and VH genes, the resulting pCMV-51-31cAbm2 plasmid facilitates the expression of a human-murine chimeric antibody (cAb) incorporating four free Cys residues, named MAb51-31cAbm2.

### C. Expression of MAb51-31cAbm2

Following confirmation by Sanger sequencing, pCMV-51-31cAbm2 was electroporated into Dux-1S-HD cells, a CHO DG44-derived cell line capable of high-density growth in serum-free medium (SFM), such as CD DG44 medium (Thermo Fisher Scientific). The cells were then plated at a density of 2×10⁵ cells/well in selection medium in 96-well tissue culture microplates. After 2-3 weeks of incubation in a CO₂ incubator, stable cell lines secreting MAb51-31cAbm2 into the culture medium were screened by ELISA and FACS analysis to assess antibody expression and binding to cancer cell lines. The selected cell line was subsequently subcloned by limited dilution, adapted to SFM, and expanded for large-scale production of MAb51-31cAbm2. MAb51-31cAbm2 purified from the culture supernatants by Protein-A affinity chromatography was analyzed by SDS-PAGE to ensure its integrity and purity.

### Example 5

### Characterization of MAb51-31cAbm2 for Binding to Cancer Cell Lines and ITGα3/β1 Protein

A. ELISA Assay of MAb51-31cAbm2 Binding to rhITGα3/β1-his

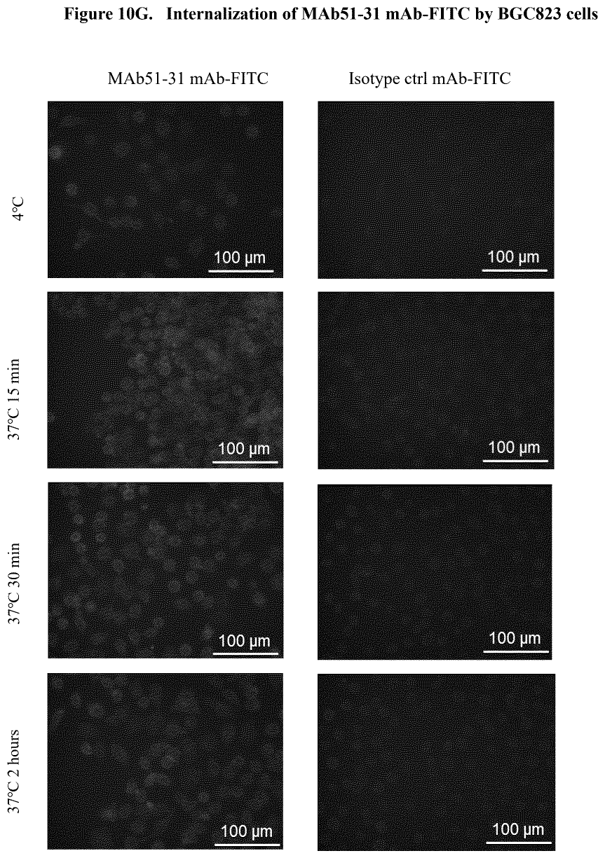

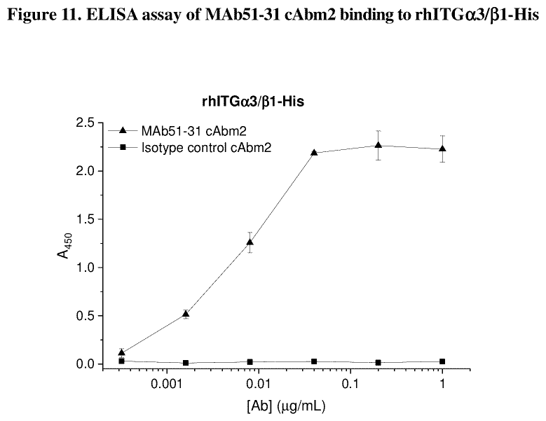

An ELISA assay was performed following the protocol described in Example 2 to evaluate the binding between MAb51-31cAbm2 and ITGα3/β1. Peroxidase AffiniPure™ Goat Anti-Human IgG, Fcγ fragment specific was used as the second antibody for detection. MAb51-31cAbm2 exhibited dose-dependent binding to rhIGTα3/β1-His, with an EC₅₀ of 0.007 μg/mL (~50 μM) (FIG. 11), demonstrating a binding pattern and affinity comparable to its mAb counterpart.

### B. FACS Analysis of MAb51-31cAbm2 Binding to Various Cancer Cell Lines

FACS analysis was conducted to assess the interaction between MAb51-31cAbm2 and various cancer cell lines, following the protocol outlined in Example 1. The only modification was the replacement of the anti-mouse IgG secondary antibody with Fluorescein AffiniPure™ Goat Anti-Human IgG, Fcγ fragment specific, to detect surface-bound cAbm2. An isotype control cAbm2, consisting of human IgG1 CH1-CH3 and human Kappa CL, served as the negative control.

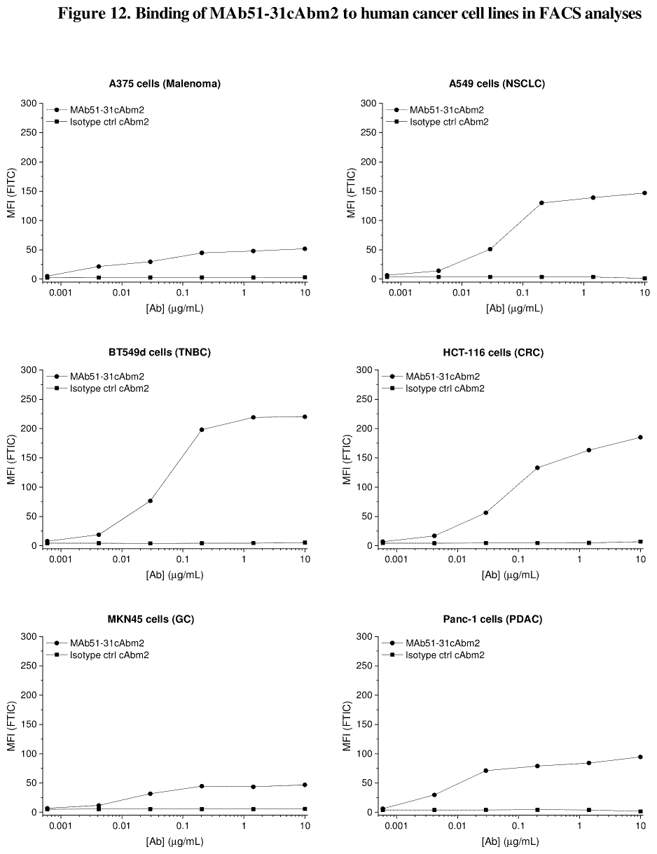

As shown in FIG. 12, MAb51-31cAbm2 exhibited dose-dependent binding to all tested solid tumor cell lines, with a binding pattern and EC₅₀ comparable to its mAb counterpart (see FIG. 1A). As expected, MAb51-31cAbm2 showed no binding to the leukemia cell lines OCI/AML2 or THP-1, which lack ITGα3/β1 expression (data not shown). This data suggests that replacement of murine constant regions with human constant regions, including point mutations, did not comprise the binding specificity of the chimeric antibody.

C. Binding Kinetics and Affinity Between MAb51-31cAbm2 and rhITGα3/β1-his

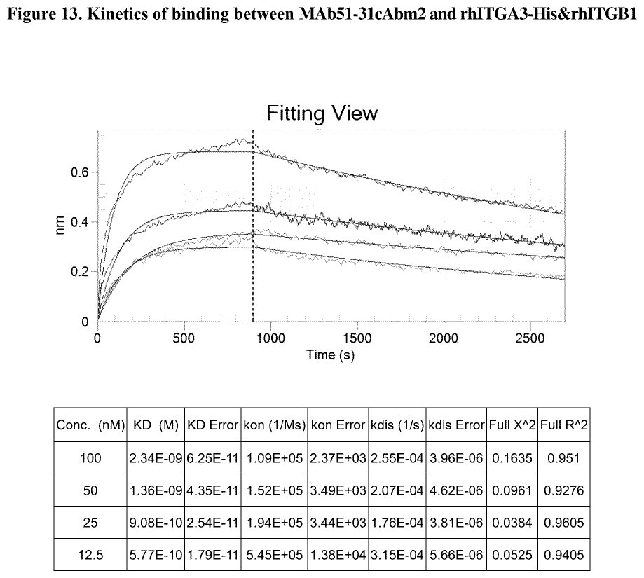

The interaction between MAb51-31cAbm2 and rhITGα3/β1-His was analyzed using the Octet® QK^{e} System equipped with Anti-Human IgG Fc Capture (AHC) Biosensors (Sartorius) following the protocol described in Example 2C. Fifty nanomolar (50 nM) MAb51-31cAbm2 was immobilized onto the AHC biosensors for 10 min, followed by a 5-min wash, a 15-min association phase with rhITGα3/β1-His, and a 30-min dissociation phase. The assay was repeated multiple times with rhITGα3/β1-His concentrations ranging from 100 nM to 12.5 nM, using 2-fold serial dilutions. A 1:1 binding model was applied to fit the binding curves. As shown in FIG. 13, MAb51-31cAbm2 exhibited a fast association rate and a moderate dissociation rate, similar to the mAb counterpart (FIG. 7), with a calculated K_{D} of approximately 1.3×10⁹ M. The affinity between MAb51-31cAbm2 and rhITGα3/β1-His appeared slightly higher than that of MAb51-31 mAb, which may be attributed to difference in the capture capacity of different biosensor types.

### Example 6

### Preparation and Binding Assays for MAb51-31cAbm2-MMAE ADC

MAb51-31 mAb demonstrated strong cancer-specificity and robust internalization capability in multiple cancer cell lines, making it a promising candidate for ADC therapy. To assess its potential as an ADC agent, MAb51-31cAbm2 and a non-cell binding isotype control cAbm2, engineered with two Cys point mutations in each IgG1 heavy chain constant region, were conjugated to a commercially available linker-payload, maleimide-valine-citrulline-PAB-monomethyl auristatin E (VcMMAE; BroadPharma, San Diego, CA). VcMMAE consists of a lysosomal-cleavable dipeptide linker (Vc), a para-aminobenzyloxycarbonyl (PABC) spacer, an anti-tubulin toxin monomethyl auristatin E (MMAE), and a thiol-reactive maleimidocaproyl (MC) group that specifically reacts with the free thiol group in cysteines. VcMMAE has been demonstrated to be an effective payload-linker in ADC in treating many types of cancer. It is a key component in FDA-approved ADCs, including Brentuximab vedotin (Adcetris®), Polatuzumab vedotin (Polivy®), Enfortumab vedotin (Padcev®), and Tisotumab vedotin (Tivdak®) (Gogia et al. Cancers, 2023; Liu et al. Mol Cancer 2024).

Site-specific conjugation was performed following the method described by Junutula et al. (Nat Biotechnol. 26:925-932, 2008). Briefly, MAb51-31cAbm2 was reduced with 1 mM dithiothreitol (DTT) to remove cysteine or glutathione adducts from the culture medium. The antibody was then refolded into its native structure in the presence of 0.4 mM dehydroascorbic acid (DHAA; MilliporeSigma), and unfolded antibody light and heavy chains were removed using an Amicon® Ultra Centrifugal Filter (50 kDa MWCO; MilliporeSigma). For conjugation, VcMMAE and MAb51-31cAbm2 were mixed at a 10:1 molar ratio with gentle agitation at RT for one hour, followed by size exclusion chromatography to remove unbound VcMMAE and potential aggregates. This site-specific conjugation protocol results in ADCs with an average drug-to-antibody ratio (DAR) of approximately 4 (DAR4).

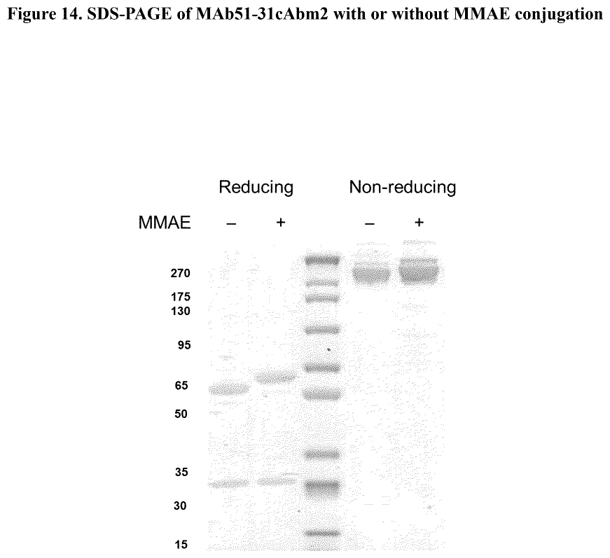

The concentration of MAb51-31cAbm2-MMAE was measured using NanoDrop™ 2000 at 280 nm and confirmed with the Pierce™ BCA Protein Assay Kit (Thermo Fisher Scientific). SDS-PAGE analysis under reducing conditions revealed distinct light and heavy chain bands, with no visible aggregation or degradation (FIG. 14). As expected, the MMAE-conjugated heavy chain band migrated higher than the unconjugated heavy chain band, indicating successful conjugation. Non-reducing SDS-PAGE showed a minor, but acceptable, fraction of aggregates.

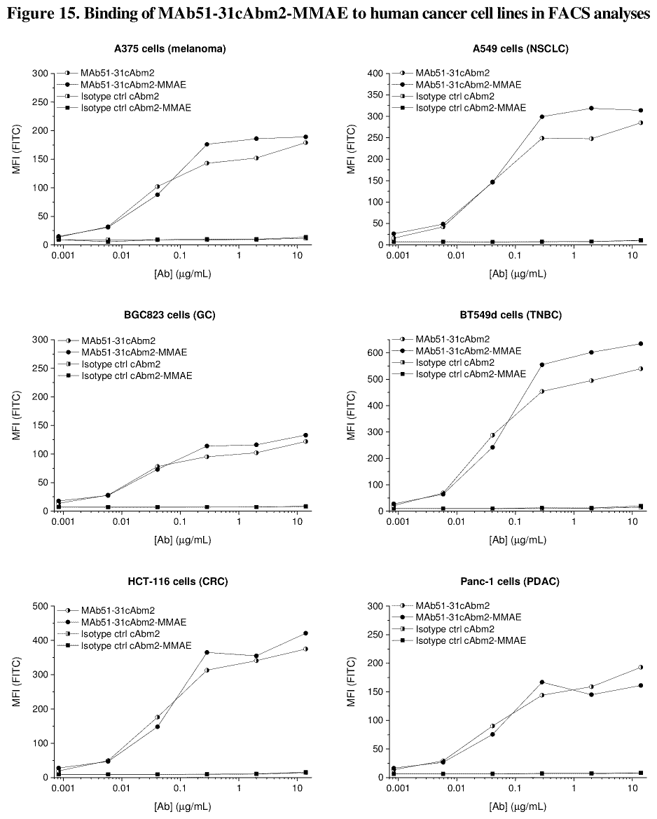

FACS analysis on surface binding to six representative cancer cell lines revealed a slight increase in FITC signals for MAb51-31cAbm2-MMAE compared to unconjugated MAb51-31cAbm2 (FIG. 15). This may be attributed to the presence of MMAE, which could affect fluorescence detection. Nonetheless, MMAE conjugation to the heavy chain constant region did not compromise the refolding or antigen-binding capacity of MAb51-31cAbm2.

### Example 7

### In Vitro Inhibitory Effects of MAb51-31cAbm2-MMAE on Cancer Cell Proliferation

The effects of MAb51-31cAbm2-MMAE on cancer cell proliferation was evaluated using cell line-based cytotoxic assays as previously described (see U.S. Pat. No. 11,939,391B2). Briefly, cancer cell lines cultured in DMEM with 10% (v/v) FBS were seeded at 3-5×10³ cells/well, depending on cell type, in triplicate in 96-well tissue culture microplates. Cells were treated with MAb51-31cAbm2-MMAE or the non-cell binding isotype control cAbm2-MMAE at 7-fold or 10-fold serial dilutions with concentrations starting from 10 μg/mL (66.7 nM). After four days of incubation at 37° C. under a 5% CO₂ humidified atmosphere, cell viability was assessed by adding 1/10 volume of the Cell Counting Kit-8 (CCK-8) solution (Dojindo Molecular Technologies, Rockville, MD) and incubating for four hours at 37° C., followed by measuring the absorbance at 450 nm (A₄₅₀).

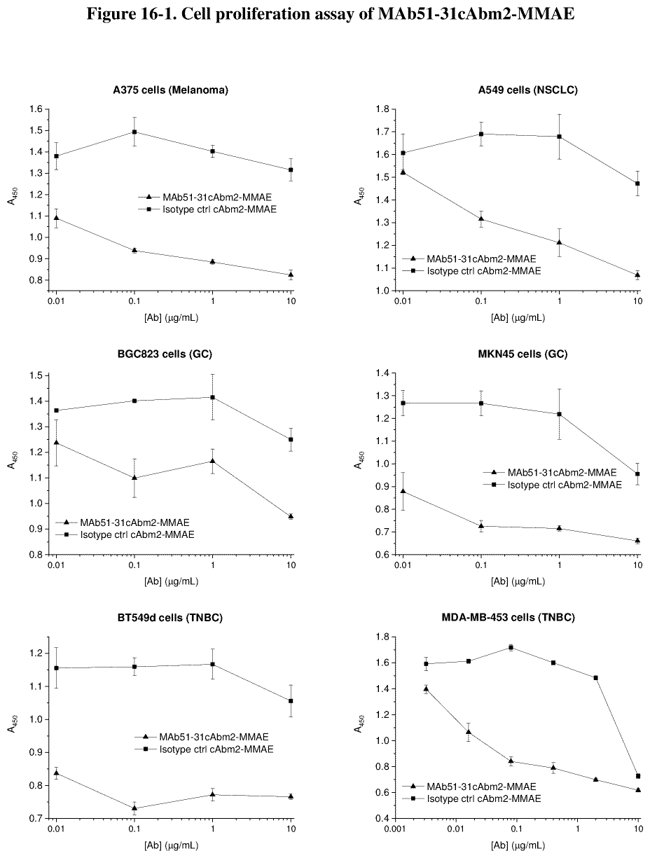

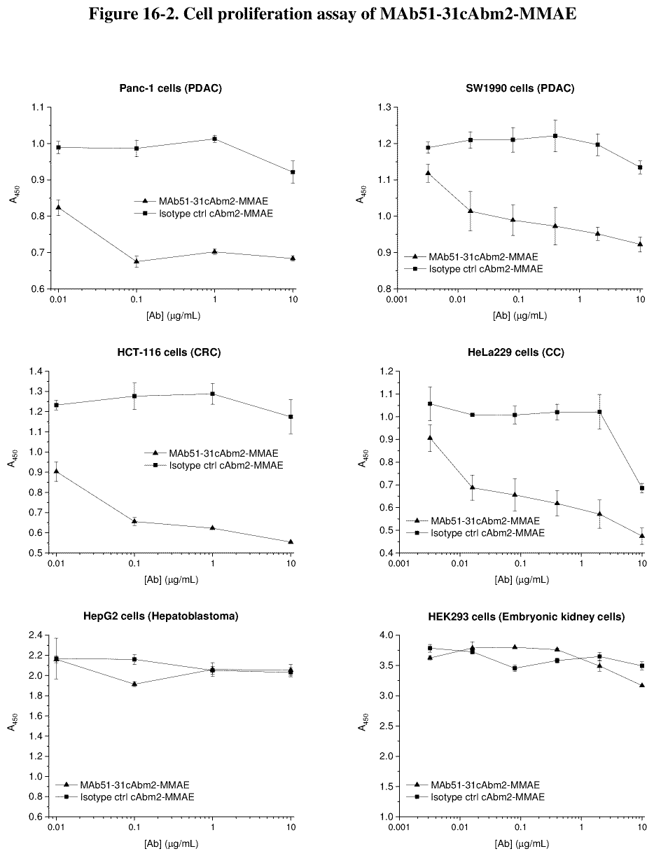

Exposure of A375, A549, BGC823 (GC; AffiGen), MKN45, BT549d, MDA-MB-453, Panc-1, SW1990, HCT-116, and HeLa229 cells to MAb51-31cAbm2-MMAE significantly reduced cell proliferation in a dose-dependent manner, whereas the isotype control ADC equipped with the same amount of MMAE exhibit no effect on the growth of either cell lines (FIG. 16). As expected, HepG2 and HEK293 cells, which did not express ITGα3/1 (see FIG. 1A), did not respond to MAb51-31cAbm2-MMAE. These results indicate that MAb51-31cAbm2-MMAE effectively suppresses cancer cell proliferation, likely due to its efficient internalization and subsequent lysosomal release of MMAE to inhibit cell division by binding to tubulin dimers and disrupting the microtubule network.

### Example 8

### In Vivo Tumor Inhibition Study of MAb51-31cAbm2-MMAE on Cancer Cell-Derived Xenograft (CDX) Mouse Models

We evaluated the antitumor effects of MAb51-31cAbm2-MMAE (DAR≈4) in six ITGα3/β1-positive cell-derived xenograft (CDX) mouse models using a single-dose treatment approach. For each model, 2×10⁶ to 5×10⁶ cancer cells, suspended in 0.1 ml of PBS, were subcutaneously (s.c.) inoculated into the flanks of 6-week-old male athymic nude mice (Charles River Laboratories, Wilmington, MA). Mice were randomized into five per treatment group when their tumor nodules were larger than 100 mm³ in size (Day 0).

Mice received intraperitoneal (i.p.) injections of either MAb51-31cAbm2-MMAE or Isotype ctrl cAbm2-MMAE (DAR≈4) at doses of 4 mg/kg, 7 mg/kg, or 10 mg/kg. In some studies, a vehicle-only control group was treated with 0.1 ml of PBS to assess potential off-target effect of the isotype control ADC. Tumor size and body weight were monitored every three to four days over four to five weeks, depending on tumor aggressiveness. Tumor volumes were calculated using the formula: Volume=½×(width)₂×length. Animals were euthanized if tumor volume exceeded 2,000 mm³ or if the tumor diameter surpassed 20 mm.

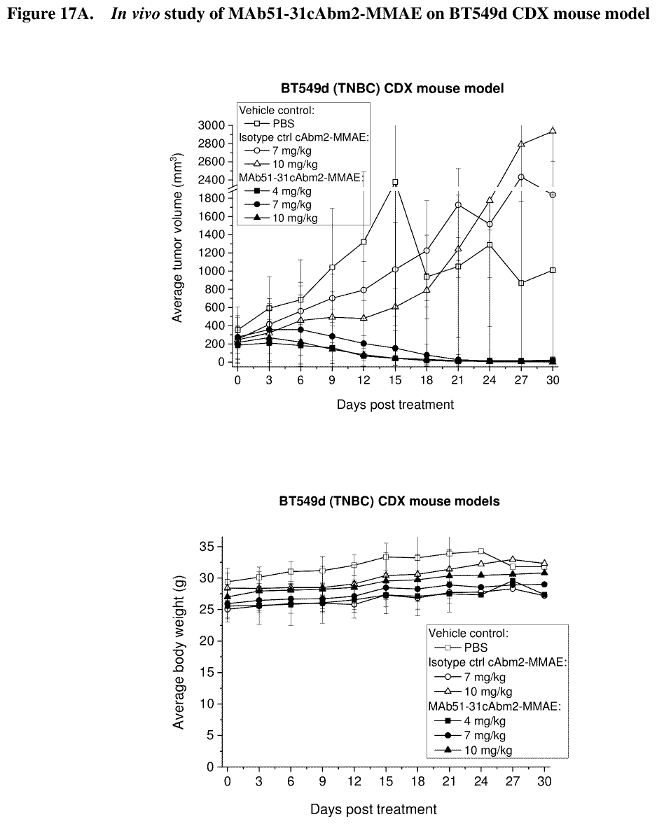

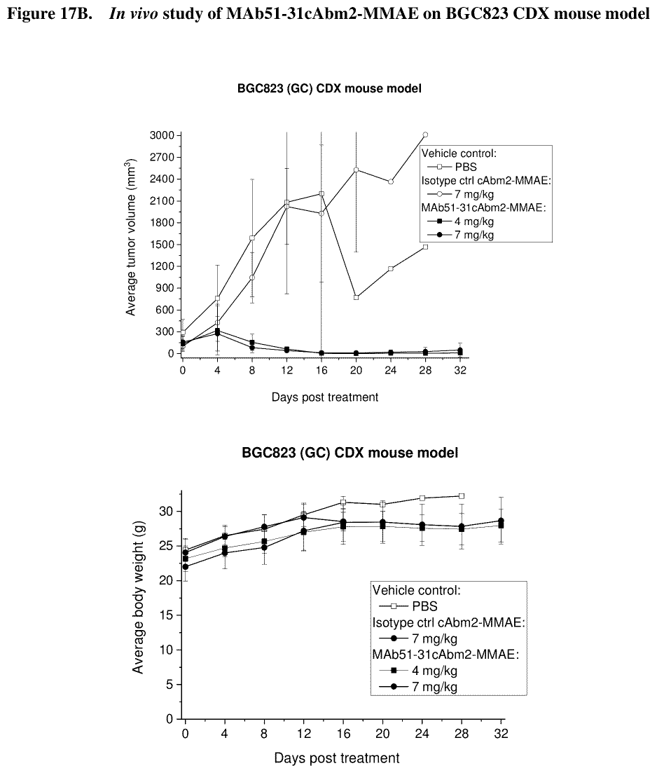

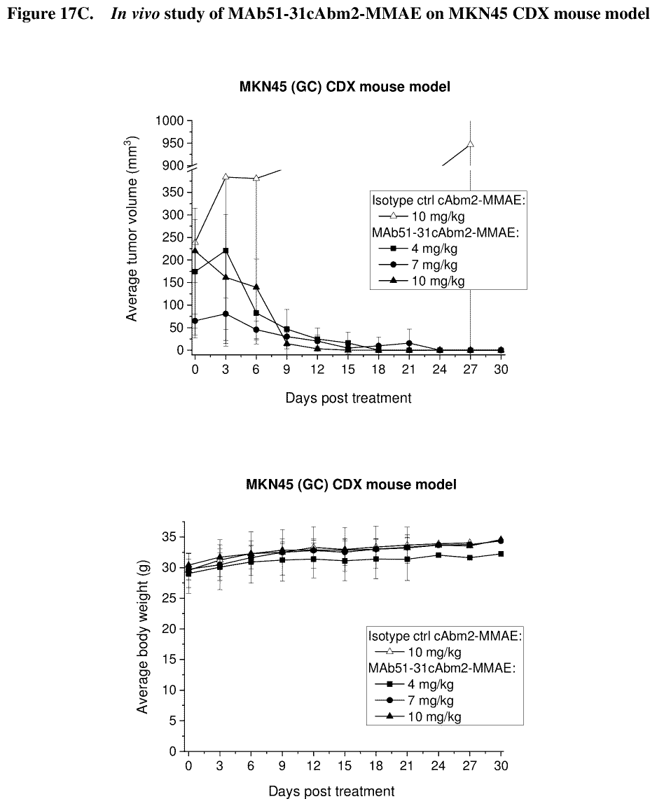

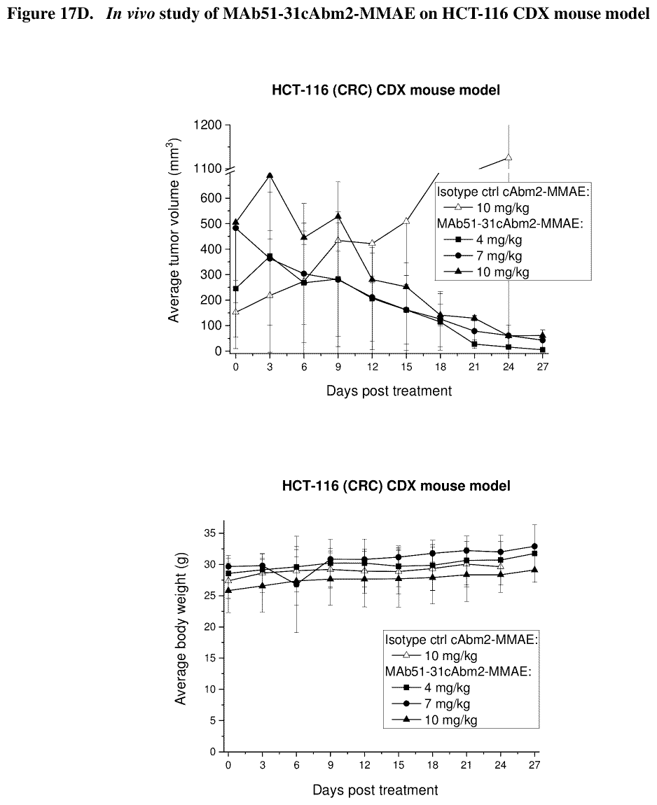

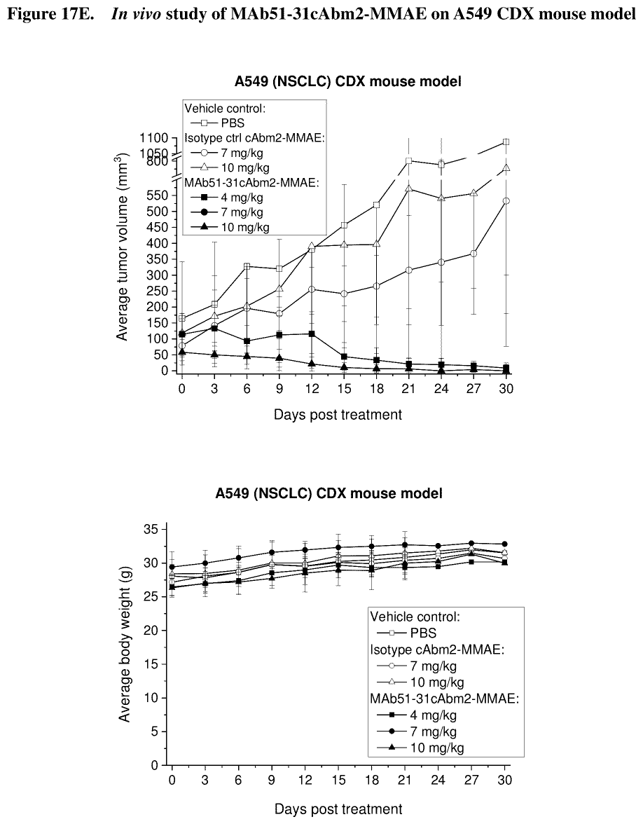

FIGS. 17A to 17F illustrate the average tumor volumes in ITGα3/1-positive BT549d (TNBC), BGC823 (GC), MKN45 (GC), HCT-116 (CRC), A549 (NSCLC), and A375 (melanoma) CDX tumor models treated with MAb51-31cAbm2-MMAE, isotype-control-MMAE, or vehicle control. Compared with vehicle-treated or isotype control cAbm2-MMAE-treated groups, MAb51-31cAbm2-MMAE significantly reduced tumor volumes in the BT549d, BGC823, MKN45, HCT-116, and A549 CDX models at all three doses (FIGS. 17A to 17E). In these models, tumor nodules halted within six to eight days post-treatment, with most tumors in the high or medium-dose groups continuing to shrink until they became invisible, implicating a robust bystander effect of the MAb51-31 ADC. In contrast, MAb51-31cAbm2-MMAE was less effective against A375 tumors since it reduced the growth rate but did not fully suppress tumor growth (FIG. 17F), indicating that this model requires multiple-dose treatment. Nevertheless, the anti-tumor effect of a single-dose MAb51-31cAbm2-MMAE administration persisted for more than three to four week across models. It is anticipated that complete tumor control with multiple-dose treatments for all these CDX models.

In three CDX models tested with a vehicle control (BT549d, A549, and A375; FIGS. 17A, 17E, and 17F), the isotype control ADC exhibited a slightly reduced tumor growth rate compared to the vehicle-treated group, suggesting a non-specific effect, potentially due to the instability of the cleavable Val-Cit-PAB linker in vivo. Although this difference was not significant, it indicates that a more stable linker may be needed for future ADC development. No adverse effects, such as weight loss, fever, lethargy, vomiting, diarrhea, bleeding, or loss of appetite, were observed in any of the MMAE-conjugated antibody-treated groups.

Similar to the in vitro assays, the varying responses of different tumor models to MAb51-31cAbm2-MMAE may be attributed to differences in surface expression of ITGα3/β1, internalization efficiency of ADC-ITGα3/β1 complex, and cellular sensitivity to MMAE. In in vivo studies, additional factors such as ADC penetration through the tumor microenvironment (TME) should also be considered.

Furthermore, a preliminary study using the non-ITGα3/β1-expressing OCI/AML2 (AML) CDX model showed no inhibitory effect by MAb51-31cAbm2-MMAE (data not shown), reinforcing that ITGα3/1 overexpression is essential for the efficacy of MAb51-31-based ADCs. Therefore, MAb51-31 mAb may serve as a companion diagnostic tool to assess biomarker expression before treatment.

### Example 9

### Humanization of MAb51-31 mAb and Verification of Target Binding for MAb51-31HuAbm2s

### A. Engineering MAb51-31 HuAbs Via CDR Grafting and Strategic Back-Mutations

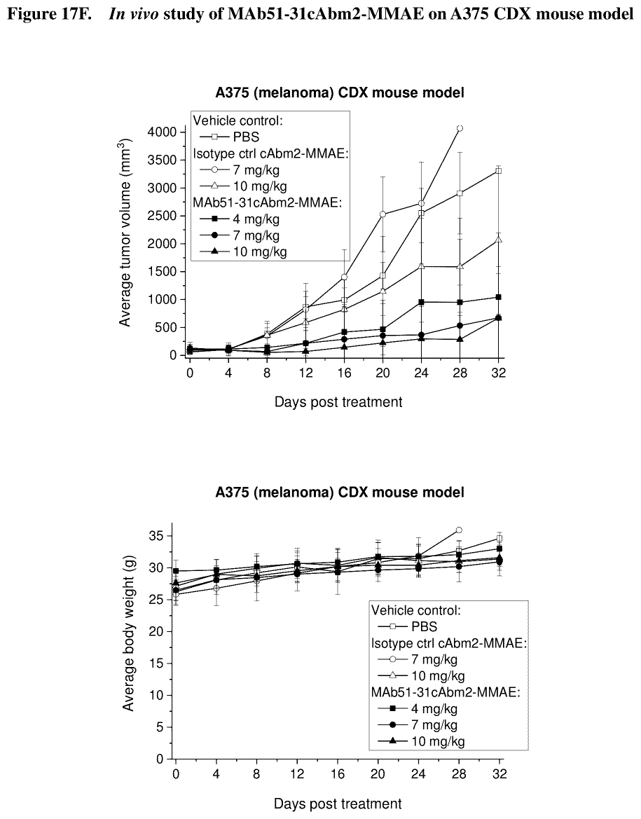

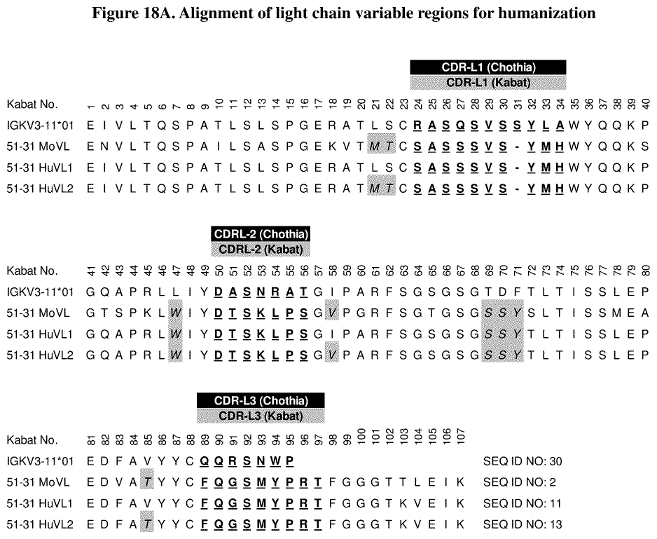

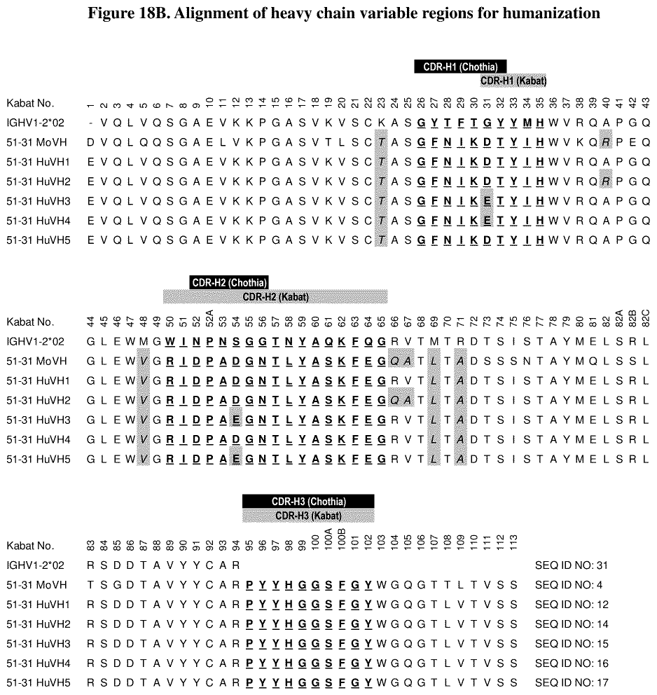

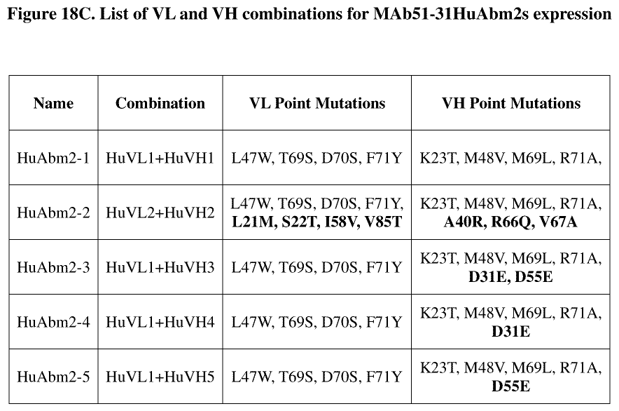

The VL (SEQ ID NO: 2) and VH (SEQ ID NO: 4) regions of murine MAb51-31 mAb were compared against human antibody germline sequences available in public databases. The optimal human germline sequences selected as the templates for humanization are shown in FIGS. 18A and 18B. Specifically, the VL frameworks (FRs) of human IGKV3-11*01 (SEQ ID NO: 30) and the VH FRs from human IGHV1-2*02 (SEQ ID NO: 31) were chosen to replace the corresponding murine FRs of MAb51-31 mAb. The sequence homologies between murine and human FRs range from 66% to 69% for VL and 57% to 71% for VH. The joint (J) region sequences selected were from human IGKJ4 (TFGGGTKVEIK, SEQ ID NO: 32) and IGHJ4 (YWGQGTLVTVSS, SEQ ID NO: 33) sequences.

To maintain proper folding while preserving antigen binding, two versions of the humanized VL and VH sequences, designated as HuVL1, HuVL2 (FIG. 18A, SEQ ID NOs: 11 and 13), HuVH1, and HuVH2 (FIG. 18B, SEQ ID NOs: 12 and 14), were designed using CDR grafting with selective back-mutations to retain the hydrophobicity, charge, steric hindrance, while minimizing antigenicity. Additionally, three more VH variants, HuVH3 (D31E and D55E), HuVH4 (D31E), and HuVH5 (D55E) (Kabat number system; FIG. 18B, SEQ ID NOs: 15, 16, and 17), were designed to substitute the aspartic acid (Asp or D) residues in the “DT” motif of CDR-H1 and the “DG” motif of CDR-H2 by glutamic acid (Glu or E) to prevent potential Asp isomerization.

The cDNA fragments encoding the humanized VL and VH variants were synthesized and annealed respectively with the human light and heavy chain constant regions encoded by the pCMV-hIgG1K-m2 expression vector following the protocol described in Example 4B. Different combinations of HuVL and HuVH variants with various back-mutations and D-to-E point mutations are listed in FIG. 18C to form five MAb51-31HuAbm2 variants, MAb51-31HuAbm2-1 to −5. All these MAb51-31HuAbm2s contained four free cysteine point mutations in the Fc regions to allow site-specific conjugation via disulfide bonds. Sanger sequencing confirmed the antibody coding sequences in these expression plasmids, which were then transfected into HEK293FT cells (Thermo Fisher Scientific) using FectoPro™ Transfection Reagent (PolyPlus Illkirch, France) according to the manufacturer's protocol. Cell cultures were maintained in 30 mL of SFM containing DMEM with 1.0% (w/v) BSA. Three days post-transfection, culture supernatants were analyzed by ELISA to quantify antibody concentrations using a capture assay with immobilized AffiniPure™ Goat Anti-Human IgG, Fcγ Fragment Specific polyclonal antibody (Jackson ImmunoResearch laboratories). Additionally, their binding capabilities to the immobilized rhITGα3/β1-His antigen were assessed. Five days post-transfection, culture supernatants were collected by centrifugation, and the humanized antibodies, MAb51-31HuAbm2s, were purified using Protein-A affinity chromatography.

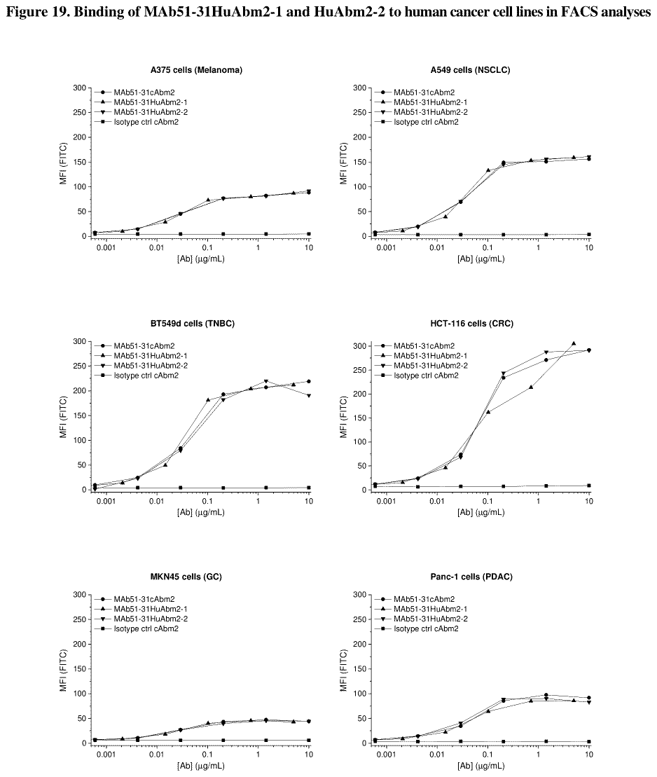

Cell staining revealed that the binding affinities of MAb51-31HuAbm2-1 and HuAbm2-2 to six representative human cancer cell lines were comparable to that of MAb51-31cAbm2 (FIG. 19), suggesting that the designed human VL and VH FRs, incorporating different back mutations, are all acceptable. Additionally, all five MAb51-31HuAbm2s exhibited similar dose-dependent binding curves to MAb51-31cAbm2 in an ELISA assay immobilized with rhIGTα3/β1-His, and no apparent band distortions indicative of Asp isomerization was observed in SDS-PAGE (data not shown). Given its minimal mutations aimed at reducing potential immunogenicity, MAb51-31HuAbm2-1 was selected to establish a stable cell line for large-scale production, following the procedure described in Example 4C, and it will be referred as MAb51-31HuAbm2 in the examples followed.

### B. Competition ELISA Assay of MAb51-31cAbm2 and MAb51-31HuAbm2 in Antigen Binding

A competition ELISA assay was performed to determine whether MAb51-31cAbm2 and MAb51-31HuAbm2 retain the ability to recognize the same epitope on rhIGTα3/β1-His as MAb51-31 mAb. In this assay, MAb51-31 mAb was conjugated with HRP using Lightning-Link® HRP Conjugation Kit as described in Example 2A. The titer of MAb51-31mAb-HRP was determined in an ELISA assay using plates coated with rhIGTα3/β1-His. Subsequently, unconjugated MAb51-31 mAb, MAb51-31cAbm2, MAb51-31HuAbm2, or isotype control cAbm2 were serially diluted in three folds, mixed with 1:6,000 diluted MAb51-31mAb-HRP, and incubated with immobilized rhIGTα3/β1-His for one hour. After thorough wash, TMB substrate was added to detect binding signals between MAb51-31mAb-HRP and the antigen.

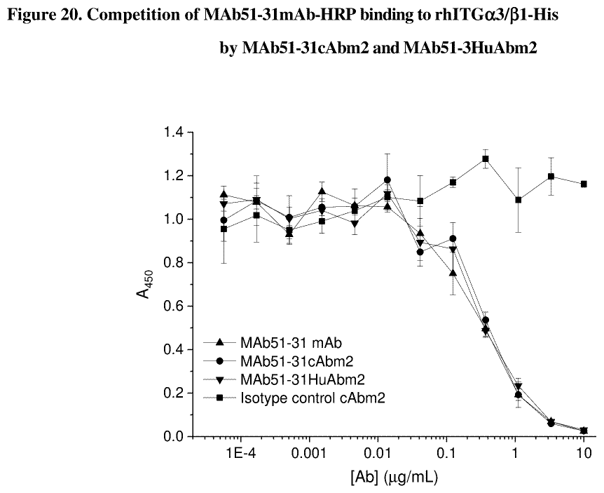

As shown in FIG. 20, the isotype control cAbm2, which targets an irrelevant antigen, served as the baseline for MAb51-31mAb-HRP and rhIGTα3/β1-His binding. Both MAb51-31cAbm2 and MAb51-31HuAbm2 reduced the binding signals in a similar dose-dependent manner as MAb51-31 mAb, with their curves nearly overlapped. This suggests that they recognize overlapping or identical epitopes on rhIGTα3/β1-His with comparable apparent affinities. This further indicates that replacing the murine antibody constant regions with human heavy chain constant regions containing Cys point mutations did not impact epitope recognition. However, additional structural and kinetic analyses would be required to confirm complete epitope identity and binding affinity equivalence.

C. Binding Kinetics and Affinity Between MAb51-31HuAbm2 and rhITGα3/β1-his

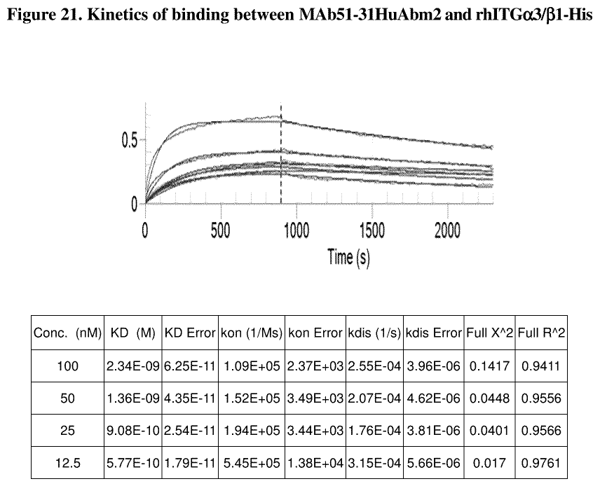

The binding kinetics and affinity between MAb51-31HuAbm2 and rhITGα3/β1-His were determined using the same protocol as for MAb51-31cAbm2. MAb51-31HuAbm2 exhibited a binding affinity constant (K_{D}) of 1.55×10-9 M (FIG. 21), which is comparable to MAb51-31cAbm2 (FIG. 13). This suggests that substituting the murine FR sequences by human FRs from IGKV3-11*01 (SEQ ID NO: 30) and IGHV1-2*02 (SEQ ID NO: 31) with the designed back mutations did not affect the binding affinity to its target.

D. Internalization Assay of MAb51-31HuAbm2-CF488 with Various Cancer Cell Lines

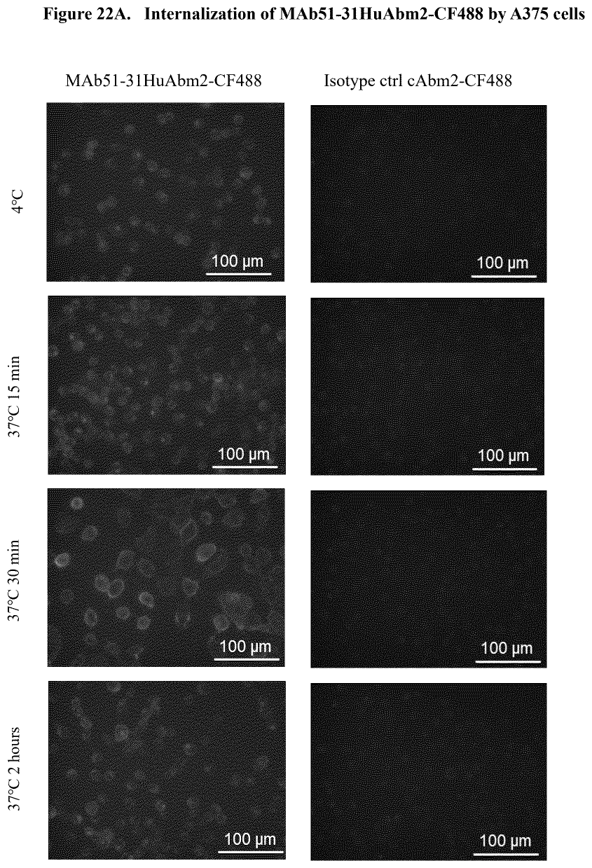

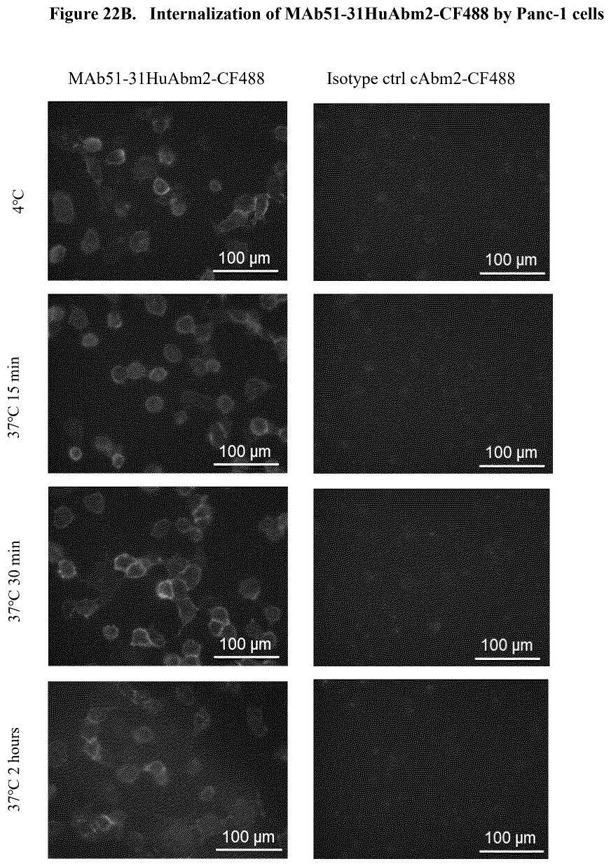

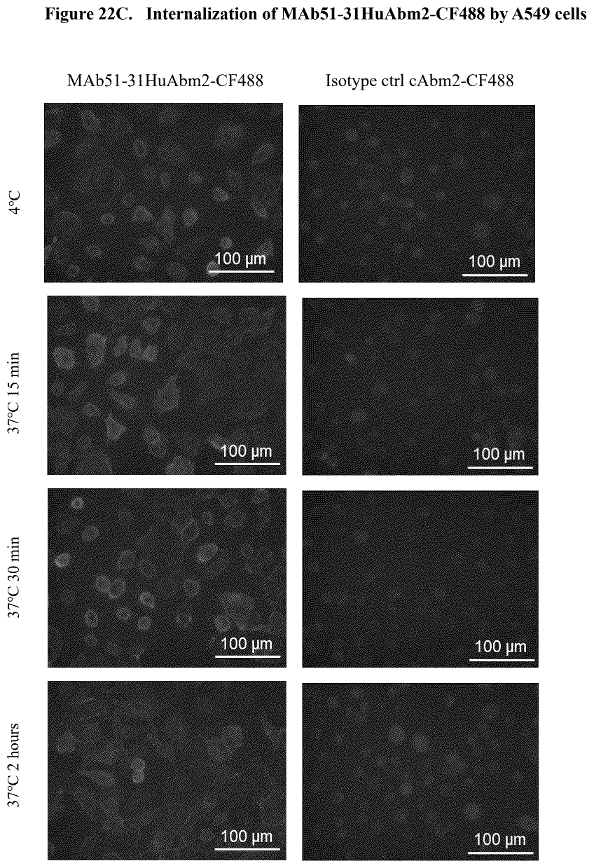

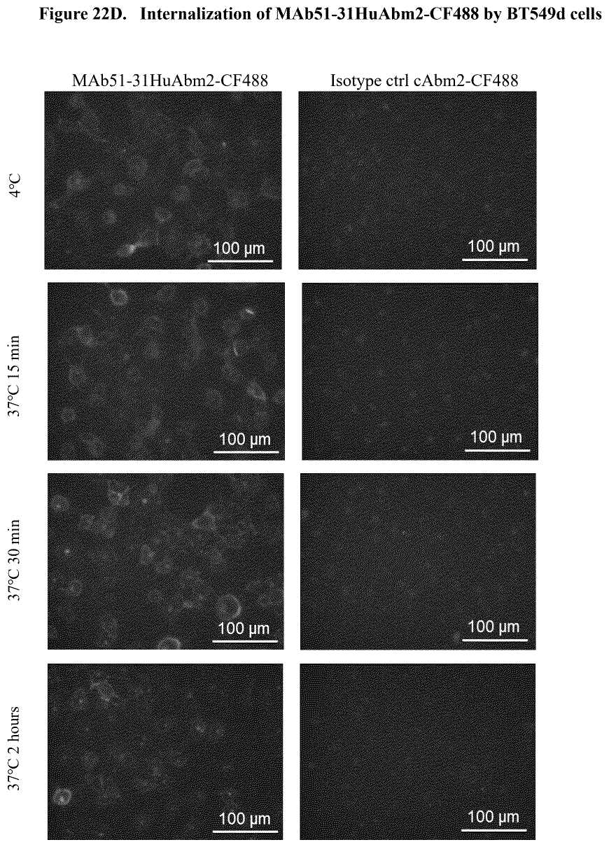

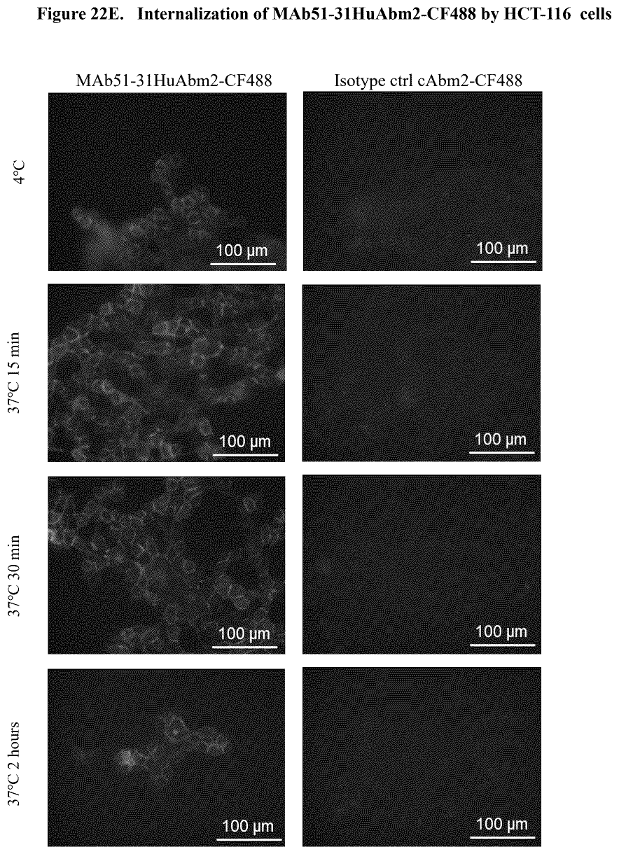

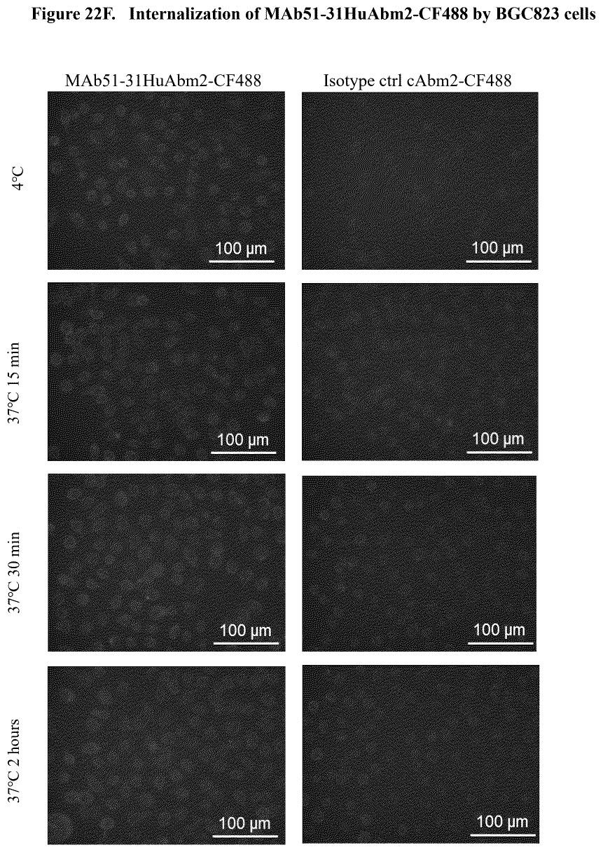

Internalization of MAb51-31HuAbm2 was evaluated following the protocol in Example 3B using CF488-labeled MAb51-31HuAbm2 or isotype control cAbm2, conjugated with the Mix-n-Stain Antibody Labeling Kit (Biotium, Fremont, CA) per the manufacturer's instructions. Consistent with MAb51-31mAb-FITC, A375, Panc-1, A549, and BT549d cells exhibited rapid and robust internalization of MAb51-31HuAbm2-CF488 (FIGS. 22A to 22D), whereas HCT-116 and BGC823 cells showed limited intracellular fluorescence (FIGS. 22E and 22F). These findings suggest that humanization preserved the internalization capability of MAb51-31HuAbm2, supporting its potential for ADC development in cancer therapy.

### Example 10

### Preparation and Characterization of MAb51-31HuAbm2 ADC

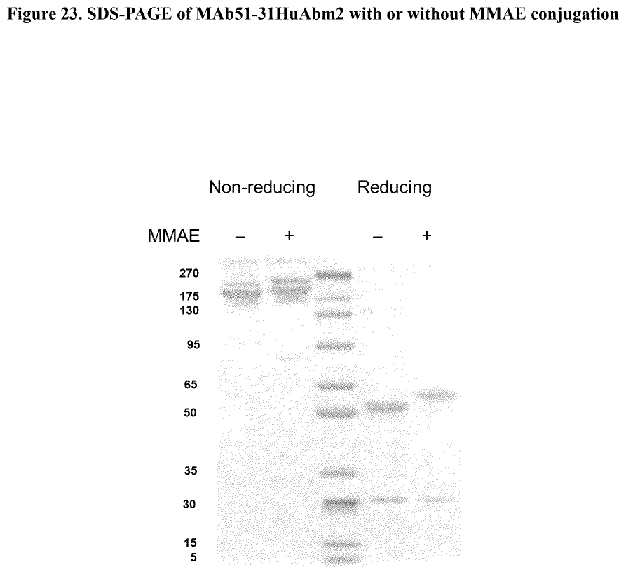

To evaluate whether the ADC derived from MAb51-31HuAbm2 has anti-cancer activity as MAb51-31cAbm2-MMAE, VcMMAE was used for site-specific conjugation following the procedure described in Example 6. MMAE was conjugated to four free cysteine residues within the heavy chain constant regions of MAb51-31HuAbm2, generating an ADC with DAR of approximately 4. SDS-PAGE analysis confirmed successful conjugation, as indicated by the apparent molecular weight shift of both the non-reduced MAb51-31HuAbm2-MMAE and the reduced heavy chain-MMAE (FIG. 23).

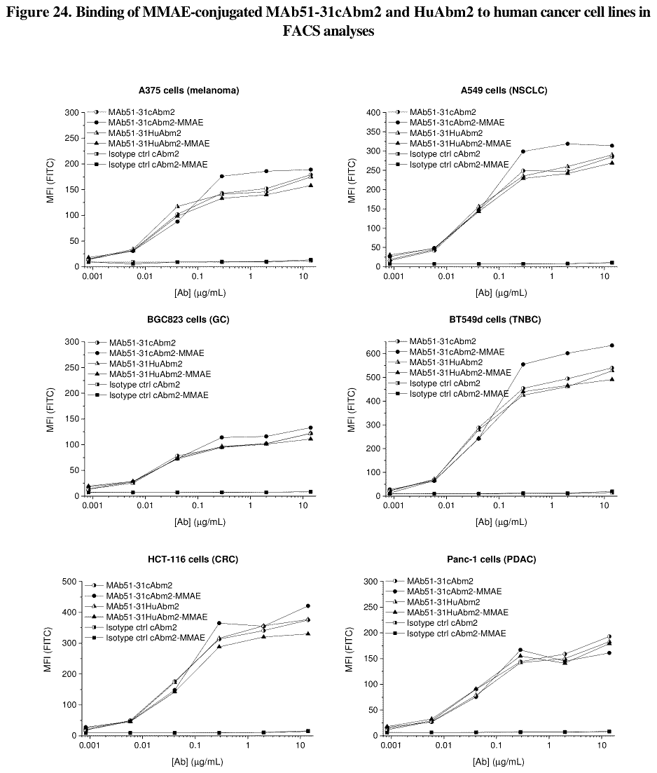

In FACS analyses, both humanized and chimeric MAb51-31 antibodies, with or without MMAE-conjugation, exhibited similar binding affinities to various cancer cell lines tested (FIG. 24). The relative MFI values, which reflect the abundance of ITGα3/β1 on different cell surface, correlated with those detected by MAb51-31 mAb (FIG. 1A) or MAb51-31cAbm2 (FIG. 12). The small variation of elevated signals by MAb51-31cAbm2-MMAE in several cell lines may be attributed by the inconsistent concentrations determined by A₂₈₀ in the presence of MMAE.

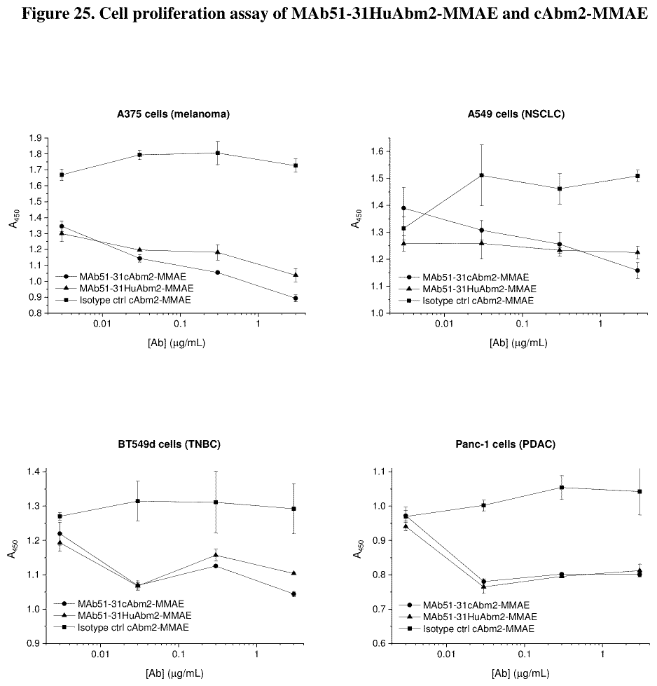

MAb51-31HuAbm2-MMAE demonstrated potent growth inhibition in A375, A549, BT549d and Panc-1 cell lines, as assessed by in vitro cell proliferation assays (FIG. 25). The magnitude of inhibition were comparable to MAb51-31cAbm2-MMAE (see FIG. 16), suggesting that ADC formulated with this humanized version of MAb51-31 mAb retains its therapeutic efficacy and can be further evaluated in in vivo PDX tumor models.

Taken together, the humanized MAb51-31HuAbm2 variants retained their binding capability to ITGα3/β1 and various human cancer cell lines. The tested ADC, MAb51-31HuAbm2-MMAE, maintained its inhibitory activity on cancer cell proliferation in vitro. Based on these findings, we conclude that MAb51-31HuAbm2 variants are likely to preserve their anti-cancer potency in vivo and are potent drug candidates for cancer therapy in human. Ongoing studies focus on optimizing the humanized variants and further evaluation of their efficacy and toxicity in preclinical animal models and clinical trials.

TABLE 1
Sequence Listing

| SEQ ID | | |
|---|---|---|
| NO: | Sequence | Note |
| 1 | AATGTTCTCACCCAGTCTCCAGCAATCTTGTCTGCATCTCCAGGGGAA | Mouse VL |
| | AAGGTCACCATGACCTGCAGTGCCAGCTCAAGTGTCAGTTACATGCAC | |
| | TGGTACCAGCAGAAGTCAGGCACCTCCCCCAAACTCTGGATTTATGAC | |
| | ACATCCAAACTGCCTTCTGGAGTCCCAGGTCGCTTCAGTGGCACTGGA | |
| | TCTGGAAGTTCTTACTCTCTCACGATCAGCAGCATGGAGGCTGAAGAT | |
| | GTTGCCACTTATTACTGTTTTCAGGGGAGTATGTATCCACGGACGTTC | |
| | GGTGGAGGCACCACGCTGGAAATCAAACGG | |
| 2 | ENVLTQSPAILSASPGEKVTMTCSASSSVSYMHWYQQKSGTSPKLWIY | Mouse VL |
| | DTSKLPSGVPGRFSGTGSGSSYSLTISSMEAEDVATYYCFQGSMYPRT | |
| | FGGGTTLEIK | |
| 3 | GACGTTCAGCTGCAGCAGTCTGGGGCAGAACTTGTGAAGCCAGGGGCC | Mouse VH |
| | TCAGTCACGTTGTCCTGCACAGCTTCTGGCTTCAACATTAAAGACACC | |
| | TATATACACTGGGTCAAACAGAGGCCTGAACAGGGCCTGGAATGGGTT | |
| | GGTAGGATTGATCCTGCGGATGGTAATACTTTATATGCCTCAAAGTTC | |
| | GAGGGCCAGGCCACTTTAACAGCAGACTCATCATCCAACACAGCCTAC | |
| | ATGCAACTCAGCAGCCTGACATCTGGGGACACTGCCGTCTATTACTGT | |
| | GCTCGCCCTTATTACCATGGTGGTAGTTTCGGCTACTGGGGCCAAGGC | |
| | ACCACTCTCACAGTCTCCTCA | |
| 4 | DVQLQQSGAELVKPGASVTLSCTASGFNIKDTYIHWVKQRPEQGLEWV | Mouse VH |
| | GRIDPADGNTLYASKFEGQATLTADSSSNTAYMQLSSLTSGDTAVYYC | |
| | ARPYYHGGSFGYWGQGTTLTVSS | |
| 5 | SASSSVSYMH | CDR-L1 |
| 6 | DTSKLPS | CDR-L2 |
| 7 | FQGSMYPRT | CDR-L3 |
| 8 | GFNIKDTYIH | CDR-H1 |
| 9 | RIDPADGNTLYASKFEG | CDR-H2 |
| 10 | PYYHGGSFGY | CDR-H3 |
| 11 | EIVLTQSPATLSLSPGERATLSCSASSSVSYMHWYQQKPGQAPRLWIY | HuVL1 |
| | DTSKLPSGIPARFSGSGSGSSYTLTISSLEPEDFAVYYCFQGSMYPRT | |
| | FGGGTKVEIK | |
| 12 | EVQLVQSGAEVKKPGASVKVSCTASGFNIKDTYIHWVRQAPGQGLEWV | HuVH1 |
| | GRIDPADGNTLYASKFEGRVTLTADTSISTAYMELSRLRSDDTAVYYC | |
| | ARPYYHGGSFGYWGQGTLVTVSS | |
| 13 | EIVLTQSPATLSLSPGERATMTCSASSSVSYMHWYQQKPGQAPRLWIY | HuVL2 |
| | DTSKLPSGVPARFSGSGSGSSYTLTISSLEPEDFATYYCFQGSMYPRT | |
| | FGGGTKVEIK | |
| 14 | EVQLVQSGAEVKKPGASVKVSCTASGFNIKDTYIHWVRQRPGQGLEWV | HuVH2 |
| | GRIDPADGNTLYASKFEGQATLTADTSISTAYMELSRLRSDDTAVYYC | |
| | ARPYYHGGSFGYWGQGTLVTVSS | |
| 15 | EVQLVQSGAEVKKPGASVKVSCTASGFNIKETYIHWVRQAPGQGLEWV | HuVH3 |
| | GRIDPAEGNTLYASKFEGRVTLTADTSISTAYMELSRLRSDDTAVYYC | |
| | ARPYYHGGSFGYWGQGTLVTVSS | |
| 16 | EVQLVQSGAEVKKPGASVKVSCTASGFNIKETYIHWVRQAPGQGLEWV | HuVH4 |
| | GRIDPADGNTLYASKFEGRVTLTADTSISTAYMELSRLRSDDTAVYYC | |
| | ARPYYHGGSFGYWGQGTLVTVSS | |
| 17 | EVQLVQSGAEVKKPGASVKVSCTASGFNIKDTYIHWVRQAPGQGLEWV | HuVH5 |
| | GRIDPAEGNTLYASKFEGRVTLTADTSISTAYMELSRLRSDDTAVYYC | |
| | ARPYYHGGSFGYWGQGTLVTVSS | |
| 18 | GFNIKETYIH | CDRH1 in HuVH3 |
| | | & 4 |
| 19 | RIDPAEGNTLYASKFEG | CDRH2 in HuVH3 |
| | | & 5 |
| 20 | TCACCAACGTGACTGTGAAG | ITGA3 Forward |
| | | primer |
| 21 | GATGCTGGTTCGGAGGAATAG | ITGA3 Reverse |
| | | primer |
| 22 | GGCGACCTGGAAGTCCAACT | RPLP0 Forward |
| | | primer |
| 23 | CCATCAGCACCACAGCCTTC | RPLP0 Reverse |
| | | primer |
| 24 | CTGCTCACTGGATGGTGGGAAGATGG | Light chain |
| | | reverse primer |
| 25 | AGCTGGGAAGGTGTGCACAC | Heavy chain |
| | | reverse primer |
| 26 | AGCACTAGTGCCGCCACCATGGATTTTCAAGTGCAG | VL Cloning |
| | | Forward primer |
| 27 | CTGGGCGCCGCTACAGTCCGTTTGATTTCCAGCGTGGTGC | VL Cloning |
| | | Reverse primer |
| 28 | AGCTCTAGAGCCGCCACCATGAAATGCAGCTGGGTTATC | VH Cloning |
| | | Forward primer |
| 29 | CTTTGGTGCTAGCTGCAGAGACTGTGAGAGTGGTGCC | VH Cloning |
| | | Reverse primer |
| 30 | EIVLTQSPATLSLSPGERATLSCRASQSVSSYLAWYQQKPGQAPRLLI | human IGKV3- |
| | YDASNRATGIPARFSGSGSGTDFTLTISSLEPEDFAVYYCQQRSNWP | 11*01 |
| 31 | VQLVQSGAEVKKPGASVKVSCKASGYTFTGYYMHWVRQAPGQGLEWMG | human IGHV1- |
| | WINPNSGGTNYAQKFQGRVTMTRDTSISTAYMELSRLRSDDTAVYYCA | 2*02 |
| | R | |
| 32 | TFGGGTKVEIK | human IGKJ4 |
| 33 | YWGQGTLVTVSS | human IGHJ4 |

### REFERENCES

Arias-Pinilla G A, Dalgleish A G, Mudan S, Bagwan I, Walker A J, Modjtahedi H (2020). Development and application of two novel mAbs against overexpressed CD26 and integrin α3 in human pancreatic cancer. Sci Rep. 10:537.
Bijnsdorp I V, Geldof A A, Lavaei M, Piersma S R, van Moorselaar R J, Jimenez C R (2013). Exosomal ITGA3 interferes with non-cancerous prostate cell functions and is increased in urine exosomes of metastatic prostate cancer patients. J Extracell Vesicles 2:22097.
Byron A, Humphries jd, Askari J A, Craig S E, Mould A P, Humphries M J (2009). Anti-integrin monoclonal antibodies. J Cell Sci. 122:4009-11.
Cegarra C, Cameron B, Chaves C, Dabdoubi T, Do™, Genêt B, Roudières V, Shi Y, Tchepikoff P, Lesuisse D (2022). An innovative strategy to identify new targets for delivering antibodies to the brain has led to the exploration of the integrin family. PLOS One. 17: e0274667.
Chen Y, Yang S, Tavormina J, Tampe D, Zeisberg M, Wang H, Mahadevan K K, Wu C J, Sugimoto H, Chang C C, Jenq R R, McAndrews K M, Kalluri R (2022). Oncogenic collagen I homotrimers from cancer cells bind to α3β1 integrin and impact tumor microbiome and immunity to promote pancreatic cancer. Cancer Cell. 40:818-834.
DiPersio C M and Longmate W M (2023). Roles for Integrin α3β1 in Development and Disease. In: Gullberg, D., Eble, J. A. (eds) Integrins in Health and Disease. Biology of Extracellular Matrix, vol 13. Springer, Cham.
Du J, Zhao Y, Hu D, Li H, Gao L, Liu Z, Shi K (2022). Silencing of integrin subunit α3 inhibits the proliferation, invasion, migration and autophagy of esophageal squamous cell carcinoma cells. Oncol Lett 24:271.
Gogia P, Ashraf H, Bhasin S, Xu Y (2023). Antibody-Drug Conjugates: A Review of Approved Drugs and Their Clinical Level of Evidence. Cancers (Basel) 15:3886.
Gullberg D and Eble J A (eds.) (2023). Integrins in Health and Disease: Key Effectors of Cell-Matrix and Cell-Cell Interactions (Biology of Extracellular Matrix, volume 13). Springer, Cham.
Islam M K, Syed P, Dhondt B, Gidwani K, Pettersson K, Lamminmäki U, Leivo J (2021). Detection of bladder cancer with aberrantly fucosylated ITGA3. Anal Biochem. 628:114283.
Jain S, Santhi P, Vinod R, Ruma S A, Huhtinen K, Pettersson K, Sundfeldt K, Leivo J, Gidwani K (2023). Aberrant glycosylation of α3 integrins as diagnostic markers in epithelial ovarian cancer. Clin Chim Acta. 543:117323.
Junutula J, Raab H, Clark S, et al. (2008). Site-specific conjugation of a cytotoxic drug to an antibody improves the therapeutic index. Nat Biotechnol. 26:925-932.
Ke F Y, Chen W Y, Lin M C, Hwang Y C, Kuo K T, Wu H C (2020). Novel monoclonal antibody against integrin α3 shows therapeutic potential for ovarian cancer. Cancer Sci. 111:3478-92.
Kim K K, Wei Y, Szekeres C, Kugler M C, Wolters P J, Hill M L, Frank J A, Brumwell A N, Wheeler S E, Kreidberg J A, Chapman H A (2009). Epithelial cell α3ß1 integrin links beta-catenin and Smad signaling to promote myofibroblast formation and pulmonary fibrosis. J Clin Invest 119:213-24.
Koshizuka K, Hanazawa T, Kikkawa N, Arai T, Okato A, Kurozumi A, Kato M, Katada K, Okamoto Y, Seki N (2017). Regulation of ITGA3 by the anti-tumor miR-199 family inhibits cancer cell migration and invasion in head and neck cancer. Cancer Sci. 108:1681-92.
Kuranaga Y, Yu B, Osuka S, Zhang H, Devi N S, Bae S, Van Meir E G (2024). Targeting Integrin α3 Blocks β1 Maturation, Triggers Endoplasmic Reticulum Stress, and Sensitizes Glioblastoma Cells to TRAIL-Mediated Apoptosis. Cells. 13:753.
Kurozumi A, Goto Y, Matsushita R, Fukumoto I, Kato M, Nishikawa R, Sakamoto S, Enokida H, Nakagawa M, Ichikawa T, Seki N (2016). Tumor-suppressive microRNA-223 inhibits cancer cell migration and invasion by targeting ITGA3/ITGB1 signaling in prostate cancer. Cancer Sci 107:84-94.
Li C, Yang Z, Du Y, Tang H, Chen J, Hu D, Fan Z (2014). BCMab1, a monoclonal antibody against aberrantly glycosylated integrin α3ß1, has potent antitumor activity of bladder cancer in vivo. Clin Cancer Res. 20:4001-13.
Li C, Yan R, Yang Z, Wang H, Zhang R, Chen H, Wang J (2017). BCMab1-Ra, a novel immunotoxin that BCMab1 antibody coupled to Ricin A chain, can eliminate bladder tumor. Oncotarget. 8:46704-5.
Linhares M M, Affonso R J, Viana L S, Silva S R M, Denadai M V A, Toledo SRC, Delcio M (2015). Genetic and Immunohistochemical Expression of Integrins ITGAV, ITGA6, and ITGA3 As Prognostic Factor for Colorectal Cancer: Models for Global and Disease-Free Survival. PLOS One 10 (12): e0144333.
Liu K, Li M, Li Y, Li Y, Chen Z, Tang Y, Yang M, Deng G, Liu H (2024). A review of the clinical efficacy of FDA-approved antibody-drug conjugates in human cancers. Mol Cancer. 23:62.
Livak K J and Schmittgen T D (2001). Analysis of relative gene expression data using real-time quantitative PCR and the 2 (−Delta Delta C (T)) method. Methods. 25:402-8.
Margadant C and Sonnenberg A (2010). Integrin-TGF-beta crosstalk in fibrosis, cancer and wound healing. EMBO Rep 11:97-105.
Ramovs V, Garcia A K, Kreft M, Sonnenberg A (2021). Integrin α3ß1 is a key regulator of several protumorigenic pathways during skin carcinogenesis. J Invest Dermatol. 141:732-741.
Schnittert J, Bansal R, Storm G, Prakash J (2018). Integrins in wound healing, fibrosis and tumor stroma: High potential targets for therapeutics and drug delivery. Adv Drug Deliv Rev 129:37-53.
Stipp C S (2010). Laminin-binding integrins and their tetraspanin partners as potential antimetastatic targets. Expert Rev Mol Med 12: e3.
Subbaram S, Dipersio C M (2011). Integrin α3ß1 as a breast cancer target. Expert Opin Ther Targets 15:1197-1210.
Takada Y, Ye X, Simon S (2007). The integrins. Genome Biol 8:215.
Tsuji T, Kawada Y, Kai-Murozono M, Komatsu S, Han S A, Takeuchi K, Mizushima H, Miyazaki K, Irimura T (2002). Regulation of melanoma cell migration and invasion by laminin-5 and α3ß1 integrin (VLA-3). Clin Exp Metastasis 19:127-34.
Zhou B, Gibson-Corley K N, Herndon M E, Sun Y, Gustafson-Wagner E, Teoh-Fitzgerald M, Domann F E, Henry M D, Stipp C S (2014). Integrin α3β1 can function to promote spontaneous metastasis and lung colonization of invasive breast carcinoma. Mol Cancer Res 12:143-154.

What is claimed is:

1. A binding agent of human integrin α3/β1 heterodimer including a light chain variable region wherein CDR1 to CDR3 have sequences at least 70% identical to, respectively, SEQ ID NO: 5, SEQ ID NO: 6, and SEQ ID NO: 7.

2. The binding agent of claim 1, wherein CDR1 to CDR3 of the light chain variable region have sequences identical to, respectively, SEQ ID NO: 5, SEQ ID NO: 6, and SEQ ID NO: 7.

3. A binding agent having a light chain variable region which is at least 70% identical to SEQ ID NO: 2.

4. A binding agent including a heavy chain variable region wherein CDR1 to CDR3 have sequences at least 70% identical to, respectively, SEQ ID NO: 8, SEQ ID NO: 9, and SEQ ID NO: 10.

5. The binding agent of claim 4, wherein CDR1 to CDR3 of the heavy chain variable region have sequences identical to, respectively, SEQ ID NO: 8, SEQ ID NO: 9, and SEQ ID NO: 10.

6. A binding agent having a heavy chain variable region which is at least 70% identical to SEQ ID NO: 4.

7. An isolated DNA or RNA molecule encoding the light chain variable region of a binding agent of claim 2.

8. An isolated DNA molecule of claim 7 having the nucleotide sequence of SEQ ID NO: 1.

9. An isolated DNA or RNA molecule encoding the heavy chain variable region of a binding agent of claim 5.

10. An isolated DNA molecule of claim 9 having the nucleotide sequence of SEQ ID NO: 3.

11. The binding agent of claim 1 or 4 wherein the binding agent is a monoclonal antibody.

12. The binding agent of claim 11 wherein the monoclonal antibody is a murine, human, humanized, chimeric, bispecific, or multispecific antibody.

13. The binding agent of claim 1 wherein the binding agent is a diabody, a single domain antigen binding (SDAB) molecule, or a VL domain.

14. The binding agent of claim 4 wherein the binding agent is a diabody, a single domain antigen binding (SDAB) molecule, a VH domain, or a VHH domain.

15. The binding agent of claim 11 wherein the monoclonal antibody has an IgG1 heavy chain and a κ light chain.

16. The binding agent of claim 1 or 4 wherein the binding agent is a Fab, Fab′, F(ab′)2, rIgG, Fv, or Fd fragment.

17. The binding agent of claim 1 or 4 wherein the binding agent is a scFv or a sc(Fv)₂.

18. The binding agent of claim 11 wherein the monoclonal antibody is class: IgD, IgE, IgG, IgA, or IgM, or a sub-class of one of said classes.

19. The monoclonal antibody of claim 18 wherein the sub-class is IgG1, IgG2, IgG3, IgG4, IgAQ1 or IgA2.

20. The monoclonal antibody of claim 18 having a κ light chain.

21. A vector comprising the isolated DNA molecules of claim 8 or 10.

22. The binding agent of claim 1 or 4 conjugated with an anti-tumor agent.

23. The binding agent of claim 22 wherein the anti-tumor agent is monomethyl auristatin E.

23. The binding agent of claim 22 wherein the conjugation is with a linker.

24. The binding agent of claim 23 wherein the linker is cleavable or non-cleavable.

25. The binding agent of claim 24 wherein the linker is a lysosomal-cleavable dipeptide linker.

26. The binding agent of claim 25 wherein the lysosomal-cleavable dipeptide linker includes a para-aminobenzyloxycarbonyl (PABC) spacer and a thiol-reactive maleimidocaproyl group that specifically reacts with a free thiol group in cysteines, as well as anti-tubulin toxin monomethyl auristatin E.

27. A binding agent of human integrin α3/β1 heterodimer including a light chain variable region having the sequences SEQ ID NO: 2, SEQ ID NO: 11, or SEQ ID NO: 13.

28. A binding agent of human integrin α3/β1 heterodimer including a heavy chain variable region having the sequences SEQ ID NO: 4, SEQ ID NO: 12, SEQ ID NO: 14, SEQ ID NO: 15, SEQ ID NO: 16, or SEQ ID NO: 17.

29. A binding agent of human integrin α3/β1 heterodimer including a light chain variable region having the sequence of SEQ ID NO: 11 and a heavy chain variable region having the sequence of SEQ ID NO: 12.

30. A binding agent of human integrin α3/β1 heterodimer including a light chain variable region having the sequence of SEQ ID NO: 2 or SEQ ID NO: 13 and a heavy chain variable region having the sequence of SEQ ID NO: 4 or SEQ ID NO: 14.


SEQUENCE LISTING (33 sequences)

SEQ ID NO: 1 - DNA, 318 bp, Mus musculus
  source 1..318: mol_type = genomic DNA; note = Mouse VL
aatgttctcacccagtctccagcaatcttgtctgcatctccaggggaaaaggtcaccatgacctgcagtgccagctcaagtgtcagttacatgcactggtaccagcagaagtcaggcacctcccccaaactctggatttatgacacatccaaactgccttctggagtcccaggtcgcttcagtggcactggatctggaagttcttactctctcacgatcagcagcatggaggctgaagatgttgccacttattactgttttcaggggagtatgtatccacggacgttcggtggaggcaccacgctggaaatcaaacgg

SEQ ID NO: 2 - protein, 106 aa, Mus musculus
  source 1..106: note = Mouse VL
ENVLTQSPAILSASPGEKVTMTCSASSSVSYMHWYQQKSGTSPKLWIYDTSKLPSGVPGRFSGTGSGSSYSLTISSMEAEDVATYYCFQGSMYPRTFGGGTTLEIK

SEQ ID NO: 3 - DNA, 357 bp, Mus musculus
  source 1..357: mol_type = genomic DNA; note = Mouse VH
gacgttcagctgcagcagtctggggcagaacttgtgaagccaggggcctcagtcacgttgtcctgcacagcttctggcttcaacattaaagacacctatatacactgggtcaaacagaggcctgaacagggcctggaatgggttggtaggattgatcctgcggatggtaatactttatatgcctcaaagttcgagggccaggccactttaacagcagactcatcatccaacacagcctacatgcaactcagcagcctgacatctggggacactgccgtctattactgtgctcgcccttattaccatggtggtagtttcggctactggggccaaggcaccactctcacagtctcctca

SEQ ID NO: 4 - protein, 119 aa, Mus musculus
  source 1..119: note = Mouse VH
DVQLQQSGAELVKPGASVTLSCTASGFNIKDTYIHWVKQRPEQGLEWVGRIDPADGNTLYASKFEGQATLTADSSSNTAYMQLSSLTSGDTAVYYCARPYYHGGSFGYWGQGTTLTVSS

SEQ ID NO: 5 - protein, 10 aa, Mus musculus
  source 1..10: note = Mouse CDR-L1
SASSSVSYMH

SEQ ID NO: 6 - protein, 7 aa, Mus musculus
  source 1..7: note = Mouse CDR-L2
DTSKLPS

SEQ ID NO: 7 - protein, 9 aa, Mus musculus
  source 1..9: note = Mouse CDR-L3
FQGSMYPRT

SEQ ID NO: 8 - protein, 10 aa, Mus musculus
  source 1..10: note = Mouse CDR-H1
GFNIKDTYIH

SEQ ID NO: 9 - protein, 17 aa, Mus musculus
  source 1..17: note = Mouse CDR-H2
RIDPADGNTLYASKFEG

SEQ ID NO: 10 - protein, 10 aa, Mus musculus
  source 1..10: note = Mouse CDR-H3
PYYHGGSFGY

SEQ ID NO: 11 - protein, 106 aa, Homo sapiens
  source 1..106: note = HuVL1
EIVLTQSPATLSLSPGERATLSCSASSSVSYMHWYQQKPGQAPRLWIYDTSKLPSGIPARFSGSGSGSSYTLTISSLEPEDFAVYYCFQGSMYPRTFGGGTKVEIK

SEQ ID NO: 12 - protein, 119 aa, Homo sapiens
  source 1..119: note = HuVH1
EVQLVQSGAEVKKPGASVKVSCTASGFNIKDTYIHWVRQAPGQGLEWVGRIDPADGNTLYASKFEGRVTLTADTSISTAYMELSRLRSDDTAVYYCARPYYHGGSFGYWGQGTLVTVSS

SEQ ID NO: 13 - protein, 106 aa, Homo sapiens
  source 1..106: note = HuVL2
EIVLTQSPATLSLSPGERATMTCSASSSVSYMHWYQQKPGQAPRLWIYDTSKLPSGVPARFSGSGSGSSYTLTISSLEPEDFATYYCFQGSMYPRTFGGGTKVEIK

SEQ ID NO: 14 - protein, 119 aa, Homo sapiens
  source 1..119: note = HuVH2
EVQLVQSGAEVKKPGASVKVSCTASGFNIKDTYIHWVRQRPGQGLEWVGRIDPADGNTLYASKFEGQATLTADTSISTAYMELSRLRSDDTAVYYCARPYYHGGSFGYWGQGTLVTVSS

SEQ ID NO: 15 - protein, 119 aa, Homo sapiens
  source 1..119: note = HuVH3
EVQLVQSGAEVKKPGASVKVSCTASGFNIKETYIHWVRQAPGQGLEWVGRIDPAEGNTLYASKFEGRVTLTADTSISTAYMELSRLRSDDTAVYYCARPYYHGGSFGYWGQGTLVTVSS

SEQ ID NO: 16 - protein, 119 aa, Homo sapiens
  source 1..119: note = HuVH4
EVQLVQSGAEVKKPGASVKVSCTASGFNIKETYIHWVRQAPGQGLEWVGRIDPADGNTLYASKFEGRVTLTADTSISTAYMELSRLRSDDTAVYYCARPYYHGGSFGYWGQGTLVTVSS

SEQ ID NO: 17 - protein, 119 aa, Homo sapiens
  source 1..119: note = HuVH5
EVQLVQSGAEVKKPGASVKVSCTASGFNIKDTYIHWVRQAPGQGLEWVGRIDPAEGNTLYASKFEGRVTLTADTSISTAYMELSRLRSDDTAVYYCARPYYHGGSFGYWGQGTLVTVSS

SEQ ID NO: 18 - protein, 10 aa, Homo sapiens
  source 1..10: note = CDRH1 in HuVH3 & 4
GFNIKETYIH

SEQ ID NO: 19 - protein, 17 aa, Homo sapiens
  source 1..17: note = CDRH2 in HuVH3 & 5
RIDPAEGNTLYASKFEG

SEQ ID NO: 20 - DNA, 20 bp, synthetic construct
  source 1..20: mol_type = other DNA; note = ITGA3 Forward primer
tcaccaacgtgactgtgaag

SEQ ID NO: 21 - DNA, 21 bp, synthetic construct
  source 1..21: mol_type = other DNA; note = ITGA3 Reverse primer
gatgctggttcggaggaatag

SEQ ID NO: 22 - DNA, 20 bp, synthetic construct
  source 1..20: mol_type = other DNA; note = RPLP0 Forward primer
ggcgacctggaagtccaact

SEQ ID NO: 23 - DNA, 20 bp, synthetic construct
  source 1..20: mol_type = other DNA; note = RPLP0 Reverse primer
ccatcagcaccacagccttc

SEQ ID NO: 24 - DNA, 26 bp, synthetic construct
  source 1..26: mol_type = other DNA; note = Light chain reverse primer
ctgctcactggatggtgggaagatgg

SEQ ID NO: 25 - DNA, 20 bp, synthetic construct
  source 1..20: mol_type = other DNA; note = Heavy chain reverse primer
agctgggaaggtgtgcacac

SEQ ID NO: 26 - DNA, 36 bp, synthetic construct
  source 1..36: mol_type = other DNA; note = VL Cloning Forward primer
agcactagtgccgccaccatggattttcaagtgcag

SEQ ID NO: 27 - DNA, 40 bp, synthetic construct
  source 1..40: mol_type = other DNA; note = VL Cloning Reverse primer
ctgggcgccgctacagtccgtttgatttccagcgtggtgc

SEQ ID NO: 28 - DNA, 39 bp, synthetic construct
  source 1..39: mol_type = other DNA; note = VH Cloning Forward primer
agctctagagccgccaccatgaaatgcagctgggttatc

SEQ ID NO: 29 - DNA, 37 bp, synthetic construct
  source 1..37: mol_type = other DNA; note = VH Cloning Reverse primer
ctttggtgctagctgcagagactgtgagagtggtgcc

SEQ ID NO: 30 - protein, 95 aa, Homo sapiens
  source 1..95: note = human IGKV3-11*01
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 31 - protein, 97 aa, Homo sapiens
  source 1..97: note = human IGHV1-2*02
VQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 32 - protein, 11 aa, Homo sapiens
  source 1..11: note = human IGKJ4
TFGGGTKVEIK

SEQ ID NO: 33 - protein, 12 aa, Homo sapiens
  source 1..12: note = human IGHJ4
YWGQGTLVTVSS